(19) United States
(12) Patent Application Publication
(10) Pub. No.: US 2025/0177285 A1
(43) Pub. Date: Jun. 5, 2025

(54) BANANA STEM SAP COMPOSITIONS AND METHODS OF USE THEREOF

(71) Applicant: Tano Pharmaceuticals Inc., Austin, TX (US)

(72) Inventors: Sean Matthew Finney, Austin, TX (US); Branson Nord Berger, Austin, TX (US)

(21) Appl. No.: 18/967,373

(22) Filed: Dec. 3, 2024

Related U.S. Application Data
(60) Provisional application No. 63/568,896, filed on Mar. 22, 2024.
(60) Provisional application No. 63/605,720, filed on Dec. 4, 2023.

(51) Int. Cl. A61K 8/9794; A61K 36/88; A61Q 19/00

(52) U.S. Cl. CPC A61K 8/9794; A61K 36/88; A61Q 19/007; A61K 2800/782

(57) ABSTRACT

Compositions comprising banana plant stem sap or extracts thereof are described. Compositions comprising banana plant stem sap or extracts thereof for topical administration to skin of a subject are described. Methods of administering the compositions for use in applications including cosmetic skincare are described. Methods of administering the compositions for use in applications including wound healing are also described.

### RELATED APPLICATIONS

This application claims the benefit of U.S. Provisional Application No. 63/605,720 filed Dec. 4, 2023, and U.S. Provisional Application No. 63/568,896 filed Mar. 22, 2024, the contents of which are herein incorporated by reference in their entireties.

### TECHNICAL FIELD

The present disclosure relates to compositions comprising banana plant stem sap or extracts thereof, and methods of administering the compositions for use in applications including cosmetic skincare or wound healing, among others.

### BACKGROUND

Portions of the banana tree (genus Musa) have long been utilized for healing purposes. In many traditional medicine systems, different parts of the banana tree have been used for their therapeutic properties. For instance, in Ayurveda, an ancient Indian system of medicine, the banana plant is revered for its healing properties. The roots, leaves, flowers, and fruit of the banana tree have been used to treat a wide range of ailments. The flowers are particularly valued for their effect on digestive disorders and menstrual pains.

However, despite ongoing interest in using portions of the banana tree for healing, there has been little to no understanding of the mechanisms underlying the beneficial effects. Furthermore, development of new formulations of banana tree products, or application of banana tree products for new uses, has been lacking.

### SUMMARY

The present disclosure relates in several embodiments to compositions comprising banana plant stem sap or extracts thereof, and methods of administering the compositions to a subject in need thereof.

According to a first aspect, the present disclosure relates in several embodiments to a cosmetic skincare composition comprising banana stem sap or an extract thereof and one or more cosmetically acceptable excipients.

The cosmetic skincare composition may include the following details, which can be combined with one another in any combinations unless clearly mutually exclusive:
(i) The cosmetic skincare composition may be formulated for topical administration.
(ii) The cosmetic skincare composition may be formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.
(iii) The banana stem sap or an extract thereof may be from one or more members of the genus Musa.
(iv) The banana stem sap or an extract thereof may be from a member of the Musa genus selected from M.×alinsanaya R. V. Valmayor, M. azizii Häkkinen, M. barioensis Häkkinen, M. bauensis Häkkinen & Meekiong, M. beccarii N. W. Simmonds, M. boman Argent, M. borneensis Becc., M. bukensis Argent, M. velutina, M. campestris Becc., M. coccinea Andrews, M. exotica R. V. Valmayor, M. fitzalanii F. Muell., M. gracilis Holttum, M. hirta Becc., M. insularimontana Hayata, M. jackeyi W. Hill, M. johnsii Argent, M. lawitiensis Nasution & Supard., M. lokok Geri & Ng, M. lolodensis Cheesman, M. maclayi F. Muell. ex Mikl.-Maclay, M. monticola M. Hotta ex Argent, M. muluensis M. Hotta, M. paracoccinea A. Z. Liu & D. Z. Li, M. peekelii Lauterb., M. salaccensis Zoll. ex Backer, M. textilis Née, M.×troglodytarum L., M. tuberculata M. Hotta, M. violascens Ridl., M. viridis R. V. Valmayor et al., M. voonii Häkkinen, M. ingens N. W. Simmonds, M. acuminata Colla, M. acuminata subsp. zebrina, M. aurantiaca G. Mann ex Baker, M. balbisiana Colla, M. banksii F. Muell., M. basjoo Siebold & Zucc. ex Iinuma, M. cheesmanii N. W. Simmonds, M. chunii Häkkinen, M. griersonii Noltie, M. itinerans Cheesman, M. laterita Cheesman, M. mannii H. Wendl. ex Baker, M. nagensium Prain, M. ochracea K. Sheph., M. ornata Roxb., Musa×paradisiaca L., M. rosea Baker, M. rubinea Häkkinen & C. H. Teo, M. rubra Wall. ex Kurz, M. sanguinea Hook .f., M. schizocarpa N. W. Simmonds, M. siamensis Häkkinen & Rich. H. Wallace, M. sikkimensis Kurz, M. thomsonii (King ex Baker) A. M. Cowan & Cowan, M. truncata Ridl., M. velutina H. Wendl. & Drude, M. yunnanensis Häkkinen & H. Wang, M. zaifui Häkkinen & H. Wang, M. arfakiana Argent, M. arunachalensis A. Joe, Sreejith & M. Sabu, M. celebica Warb. ex K. Schum., M. juwiniana Meekiong, M. kattuvazhana K. C. Jacob, M. lanceolata Warb. ex K. Schum., M. lutea R. V. Valmayor et al., M. sakaiana Meekiong et al., M. shankarii Subba Rao & Kumari, M. splendida A. Chev., M. tonkinensis R. V. Valmayor et al., and M. yamiensis C. L. Yeh & J. H. Chen, and any combinations thereof.
(v) The one or more cosmetically acceptable excipients may include one or more fragrances, essential oils, colorants, preservatives, exfoliating agents, anti-aging ingredients, mattefying agents, alcohols, silicones, hydroquinones, or combinations thereof.
(vi) The cosmetic skincare composition may include an amount of the banana stem sap or extract thereof effective to increase fibroblast proliferation in the skin, increase PDGF-BB in the skin, increase collagen production in the skin, or any combinations thereof, following administration of the composition to skin of a subject.
(vii) The cosmetic skincare composition may include an amount of the banana stem sap or extract thereof effective to reduce wrinkles in the skin, reduce fine lines in the skin, reduce skin laxity, improve skin texture, improve skin tone, reduce visibility of scars, reduce stretch marks, reduce age spots, reduce hyperpigmentation, reduce skin roughness, reduce uneven pigmentation, reduce dullness, increase skin hydration, increase skin elasticity, increase skin firmness, reduce enlarged skin pore size, reduce acne, reduce acne scars, reduce skin damage caused by sun exposure, reduce skin damage caused by exposure to pollution, reduce under-eye skin circles, reduce under-eye skin bags, reduce skin puffiness, increase skin radiance, increase skin glow, reduce skin irritation, reduce skin rashes (e.g. diaper rashes, among others), reduce skin blemishes, promote skin tightening, reduce eczema, or any combinations thereof, following administration of the composition to skin of a subject.

According to a second aspect, the present disclosure relates in several embodiments to a method of treating or preventing a cosmetic skin condition in a subject. The method includes administering to the subject's skin an effective amount of banana stem sap or an extract thereof, or an effective amount of a cosmetic skincare composition described herein.

In some embodiments of the method, the subject's skin to which the cosmetic skincare composition is administered does not comprise a wound.

According to a third aspect, the present disclosure relates in several embodiments to a wound healing composition comprising banana stem sap or an extract thereof and one or more pharmaceutically acceptable excipients.

The wound healing composition may include the following details, which can be combined with one another in any combinations unless clearly mutually exclusive:
(i) The wound healing composition may be formulated for topical administration.
(ii) The wound healing composition may be formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.
(iii) The banana stem sap or an extract thereof may be from one or more members of the genus Musa.
(iv) The banana stem sap or an extract thereof may be from a member of the Musa genus selected from M.×alinsanaya R. V. Valmayor, M. azizii Häkkinen, M. barioensis Häkkinen, M. bauensis Häkkinen & Meekiong, M. beccarii N. W. Simmonds, M. boman Argent, M. borneensis Becc., M. bukensis Argent, M. velutina, M. campestris Becc., M. coccinea Andrews, M. exotica R. V. Valmayor, M. fitzalanii F. Muell., M. gracilis Holttum, M. hirta Becc., M. insularimontana Hayata, M. jackeyi W. Hill, M. johnsii Argent, M. lawitiensis Nasution & Supard., M. lokok Geri & Ng, M. lolodensis Cheesman, M. maclayi F. Muell. ex Mikl.-Maclay, M. monticola M. Hotta ex Argent, M. muluensis M. Hotta, M. paracoccinea A. Z. Liu & D. Z. Li, M. peekelii Lauterb., M. salaccensis Zoll. ex Backer, M. textilis Née, M.×troglodytarum L., M. tuberculata M. Hotta, M. violascens Ridl., M. viridis R. V. Valmayor et al., M. voonii Häkkinen, M. ingens N. W. Simmonds, M. acuminata Colla, M. acuminata subsp. zebrina, M. aurantiaca G. Mann ex Baker, M. balbisiana Colla, M. banksii F. Muell., M. basjoo Siebold & Zucc. ex Iinuma, M. cheesmanii N. W. Simmonds, M. chunii Häkkinen, M. griersonii Noltie, M. itinerans Cheesman, M. laterita Cheesman, M. mannii H. Wendl. ex Baker, M. nagensium Prain, M. ochracea K. Sheph., M. ornata Roxb., Musa×paradisiaca L., M. rosea Baker, M. rubinea Häkkinen & C. H. Teo, M. rubra Wall. ex Kurz, M. sanguinea Hook.f., M. schizocarpa N. W. Simmonds, M. siamensis Häkkinen & Rich. H. Wallace, M. sikkimensis Kurz, M. thomsonii (King ex Baker) A. M. Cowan & Cowan, M. truncata Ridl., M. velutina H. Wendl. & Drude, M. yunnanensis Häkkinen & H. Wang, M. zaifui Häkkinen & H. Wang, M. arfakiana Argent, M. arunachalensis A. Joe, Sreejith & M. Sabu, M. celebica Warb. ex K. Schum., M. juwiniana Meekiong, M. kattuvazhana K. C. Jacob, M. lanceolata Warb. ex K. Schum., M. lutea R. V. Valmayor et al., M. sakaiana Meekiong et al., M. shankarii Subba Rao & Kumari, M. splendida A. Chev., M. tonkinensis R. V. Valmayor et al., and M. yamiensis C. L. Yeh & J. H. Chen, and any combinations thereof.
(v) The wound healing composition may include an amount of the banana stem sap or extract thereof effective to increase fibroblast proliferation in the skin, increase PDGF-BB in the skin, increase collagen production in the skin, or any combinations thereof, following administration of the composition to a wound in the skin of the subject.
(vi) The wound healing composition may include an amount of the banana stem sap or extract thereof effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems, improve in the healing of skin and tissue damage caused by radiation treatments, reduce scar formation, or any combination thereof, following administration of the composition to a wound in skin of a subject.
(vii) The wound may be an acute wound, a chronic wound, a cut, an abrasion, a surgical incision, a skin graft, a traumatic wound, a hypertrophic scar, a diabetic ulcer, a venous ulcer, a pressure sore, a surgical wound, a post-operative wound, a burn wound, skin or tissue damage caused by radiation treatments, or any combination thereof.

According to a fourth aspect, the present disclosure relates in several embodiments to a method of treating a subject for a wound. The method includes administering to the subject's skin an effective amount of a wound healing composition described herein.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

The present disclosure may be further understood through reference to the attached figures in combination with the detailed description that follows.

FIG. 1 is a photomicrograph of HaCaT keratinocyte culture. The photomicrograph was acquired with the EVOS 5000 microscope (ThermoFisher Scientific, Waltham, MA) at final mag. ×40).

FIG. 2 is a list of genes from the interrogated array, which were significantly modulated by UVB irradiation of HaCaT keratinocytes 24 h after irradiation.

FIG. 3 is a list of genes from the interrogated array, which were significantly modulated by UVB irradiation of HaCaT keratinocytes pretreated by B.E. vs. the water-treated controls, 24 h after irradiation.

FIG. 4A is a graph reporting example data on the effect of test article MB-2024 at the indicated concentrations on collagenase (MP) enzymatic activity as a function of time, expressed as arbitrary fluorescence units. Bars: standard error of the mean.

FIG. 4B is a graph reporting example data on the effect of test article DBF-2024 at the indicated concentrations on collagenase (MP) enzymatic activity as a function of time, expressed as arbitrary fluorescence units. Bars: standard error of the mean.

FIG. 5 is a graph reporting example results of differential gene expression between treatment groups. A total of 21,448 annotated Human Genes were filtered using fold change: >1 or <−1 and p Value <0.05 using one-way ANOVA (empirical Bayes method).

### DETAILED DESCRIPTION

The present disclosure relates in several embodiments to compositions comprising banana plant stem sap or extracts thereof. In several embodiments, such compositions are formulated for topical administration to skin of a subject. In several embodiments, the present disclosure relates to methods of administering the compositions for cosmetic skincare. In other embodiments, the present disclosure relates to methods of administering the compositions for wound healing.

In some embodiments, the present disclosure relates to a cosmetic skincare composition comprising banana stem sap or an extract thereof and one or more cosmetically acceptable excipients.

As used herein, the term “cosmetic skincare composition” relates to a composition intended to be applied to the skin of a subject for purposes including, without limitation, cleansing, beautifying, promoting attractiveness, altering the appearance, or any combinations thereof. In some embodiments, a cosmetic skincare composition of the present disclosure is not intended for use in treatment of a wound. In some embodiments, a cosmetic skincare composition of the present disclosure may also be suitable in treatment of a wound. In some embodiments, a cosmetic skincare composition of the present disclosure is not suitable for use in treatment of a wound.

In some embodiments, the cosmetic skincare composition comprises banana stem sap or an extract thereof from any species of the genus Musa. It is believed that banana sap or an extract thereof has not been used previously in cosmetic formulations.

The genus Musa comprises a diverse group of flowering plants commonly known as bananas and plantains. The term “banana” as used herein includes both bananas and plantains in the genus Musa. The genus Musa belongs to the family Musaceae and is characterized by large, herbaceous plants with pseudostems formed by tightly packed leaf bases. The genus Musa comprises various species and subspecies including, but not limited to, the following. Musa acuminata (also known as wild banana), includes subspecies such as Musa acuminata subsp. acuminata (also known as sweet or dessert banana) which includes varieties such as Cavendish and Gros Michel. Musa acuminata also includes subspecies Musa acuminata subsp. Burmannicoides, which are wild bananas with seeds, not typically consumed. Musa balbisiana (also known as Plantains) includes subspecies such as Musa balbisiana subsp. Balbisiana, which are a wild species with seeds and starchy fruit, mainly used for cooking. Musa balbisiana also includes subspecies such as Musa balbisiana subsp. banksia, also known as the Papua New Guinea Highland banana. Various hybrids of Musa species are known, including for example, and not by limitation, Musa×paradisiaca (also known as Dwarf Blue Field Banana), which is a hybrid between Musa acuminata and Musa balbisiana, includes various plantains and cooking bananas. Various cultivated varieties are known, including but not limited to Musa ‘Dwarf Cavendish’, a popular, smaller variety of banana; Musa ‘Grand Nain’ (also known as Chiquita Banana), a type of dessert banana; Musa ‘Lady Finger’ (also known as Sucrier), a smaller, sweet banana; Musa ‘Red’ (also known as Red Banana), known for its reddish-purple skin and sweet taste. Ornamental varieties include but are not limited to Musa ornata (also known as Flowering Banana), grown for its attractive flowers rather than fruit; and Musa velutina (also known as Pink Banana), a small banana with pink skin and fruit.

In some embodiments, the banana species may be Musa Balbisiana, Musa Acuminata or any diploid, triploid or tetraploid derived therefrom, or any combinations thereof.

In some embodiments, members of the Musa genus may include the following, without limitation: M.×alinsanaya R. V. Valmayor, M. azizii Häkkinen, M. barioensis Häkkinen, M. bauensis Häkkinen & Meekiong, M. beccarii N. W. Simmonds, M. boman Argent, M. borneensis Becc., M. bukensis Argent, M. velutina, M. campestris Becc., M. coccinea Andrews, M. exotica R. V. Valmayor, M. fitzalanii F. Muell., M. gracilis Holttum, M. hirta Becc., M. insularimontana Hayata, M. jackeyi W. Hill, M. johnsii Argent, M. lawitiensis Nasution & Supard., M. lokok Geri & Ng, M. lolodensis Cheesman, M. maclayi F. Muell. ex Mikl.-Maclay, M. monticola M. Hotta ex Argent, M. muluensis M. Hotta, M. paracoccinea A. Z. Liu & D. Z. Li, M. peekelii Lauterb., M. salaccensis Zoll. ex Backer, M. textilis Née, M.×troglodytarum L., M. tuberculata M. Hotta, M. violascens Ridl., M. viridis R. V. Valmayor et al., M. voonii Häkkinen, M. ingens N. W. Simmonds, M. acuminata Colla, M. acuminata subsp. zebrina, M. aurantiaca G. Mann ex Baker, M. balbisiana Colla, M. banksii F. Muell., M. basjoo Siebold & Zucc. ex Iinuma, M. cheesmanii N. W. Simmonds, M. chunii Häkkinen, M. griersonii Noltie, M. itinerans Cheesman, M. laterita Cheesman, M. mannii H. Wendl. ex Baker, M. nagensium Prain, M. ochracea K. Sheph., M. ornata Roxb., Musa×paradisiaca L., M. rosea Baker, M. rubinea Häkkinen & C. H. Teo, M. rubra Wall. ex Kurz, M. sanguinea Hook.f., M. schizocarpa N. W. Simmonds, M. siamensis Häkkinen & Rich. H. Wallace, M. sikkimensis Kurz, M. thomsonii (King ex Baker) A. M. Cowan & Cowan, M. truncata Ridl., M. velutina H. Wendl. & Drude, M. yunnanensis Häkkinen & H. Wang, M. zaifui Häkkinen & H. Wang, M. arfakiana Argent, M. arunachalensis A. Joe, Sreejith & M. Sabu, M. celebica Warb. ex K. Schum., M. juwiniana Meekiong, M. kattuvazhana K. C. Jacob, M. lanceolata Warb. ex K. Schum., M. lutea R. V. Valmayor et al., M. sakaiana Meekiong et al., M. shankarii Subba Rao & Kumari, M. splendida A. Chev., M. tonkinensis R. V. Valmayor et al., and M. yamiensis C. L. Yeh & J. H. Chen, among other members of the Musa genus identifiable by skilled persons in view of the present disclosure.

In some embodiments, the banana stem sap may be obtained from one or more different members of the genus Musa.

Banana stem sap, also known as banana juice or banana water, refers to the liquid that may be obtained or extracted from the stem of the banana plant. The stem sap may be obtained by cutting the stem and allowing the liquid to drain out. The sap is typically obtained by making a cut or incision in the banana plant's pseudostem, which is a portion of the plant having the appearance of a trunk but is composed of tightly packed leaf bases. Banana stem sap typically has a clear, slightly green, or slightly milky liquid appearance. Extraction of banana stem sap may be performed by hand, by using suitable mechanical extraction methods known in the art, or combinations thereof.

Following extraction, the banana stem sap may be used fresh, stored (e.g., at room temperature or refrigerated) until use. Following extraction, the banana stem sap may be processed according to any method known in the art. For example, in some embodiments the banana stem sap may be cooked, fermented, concentrated, dried, powdered, preserved, or any combinations thereof.

For example, banana stem sap can be concentrated to create a syrup or thick liquid. The sap may be simmered or boiled to reduce its water content, resulting in a thicker consistency. Banana stem sap can be dried and processed into a powder. The sap may be concentrated using a rotary evaporator. The sap may be filtered, then lyophilized. The sap may be spread thinly and allowed to air-dry or may be dehydrated. Once dried, it is ground into a fine powder. Banana stem sap can be preserved for longer shelf life. This can involve adding preservatives, refrigeration, or both.

In some embodiments, the cosmetic skincare compositions and wound healing compositions described herein may include banana stem sap or an extract thereof in an amount of, or in an amount of about, 0.01% to 100% by weight (w/w). In some embodiments, the cosmetic skincare compositions and wound healing compositions described herein may include banana stem sap or an extract thereof in an amount by weight (w/w) of, or in an amount by weight (w/w) of about, 0.01%, 0.02%, 0.04%, 0.06%, 0.08%, 0.1%, 0.2%, 0.4%, 0.6%, 0.8%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.6%, 8.8%, 9.0%, 9.2%, 9.4%, 9.6%, 9.8%, 10.0%, 10.2%, 10.4%, 10.6%, 10.8%, 11.0%, 11.2%, 11.4%, 11.6%, 11.8%, 12.0%, 12.2%, 12.4%, 12.6%, 12.8%, 13.0%, 13.2%, 13.4%, 13.6%, 13.8%, 14.0%, 14.2%, 14.4%, 14.6%, 14.8%, 15.0%, 15.2%, 15.4%, 15.6%, 15.8%, 16.0%, 16.2%, 16.4%, 16.6%, 16.8%, 17.0%, 17.2%, 17.4%, 17.6%, 17.8%, 18.0%, 18.2%, 18.4%, 18.6%, 18.8%, 19.0%, 19.2%, 19.4%, 19.6%, 19.8%, 20.0%, 20.2%, 20.4%, 20.6%, 20.8%, 21.0%, 21.2%, 21.4%, 21.6%, 21.8%, 22.0%, 22.2%, 22.4%, 22.6%, 22.8%, 23.0%, 23.2%, 23.4%, 23.6%, 23.8%, 24.0%, 24.2%, 24.4%, 24.6%, 24.8%, 25.0%, 25.2%, 25.4%, 25.6%, 25.8%, 26.0%, 26.2%, 26.4%, 26.6%, 26.8%, 27.0%, 27.2%, 27.4%, 27.6%, 27.8%, 28.0%, 28.2%, 28.4%, 28.6%, 28.8%, 29.0%, 29.2%, 29.4%, 29.6%, 29.8%, 30.0%, 30.2%, 30.4%, 30.6%, 30.8%, 31.0%, 31.2%, 31.4%, 31.6%, 31.8%, 32.0%, 32.2%, 32.4%, 32.6%, 32.8%, 33.0%, 33.2%, 33.4%, 33.6%, 33.8%, 34.0%, 34.2%, 34.4%, 34.6%, 34.8%, 35.0%, 35.2%, 35.4%, 35.6%, 35.8%, 36.0%, 36.2%, 36.4%, 36.6%, 36.8%, 37.0%, 37.2%, 37.4%, 37.6%, 37.8%, 38.0%, 38.2%, 38.4%, 38.6%, 38.8%, 39.0%, 39.2%, 39.4%, 39.6%, 39.8%, 40.0%, 40.2%, 40.4%, 40.6%, 40.8%, 41.0%, 41.2%, 41.4%, 41.6%, 41.8%, 42.0%, 42.2%, 42.4%, 42.6%, 42.8%, 43.0%, 43.2%, 43.4%, 43.6%, 43.8%, 44.0%, 44.2%, 44.4%, 44.6%, 44.8%, 45.0%, 45.2%, 45.4%, 45.6%, 45.8%, 46.0%, 46.2%, 46.4%, 46.6%, 46.8%, 47.0%, 47.2%, 47.4%, 47.6%, 47.8%, 48.0%, 48.2%, 48.4%, 48.6%, 48.8%, 49.0%, 49.2%, 49.4%, 49.6%, 49.8%, 50.0%, 50.2%, 50.4%, 50.6%, 50.8%, 51.0%, 51.2%, 51.4%, 51.6%, 51.8%, 52.0%, 52.2%, 52.4%, 52.6%, 52.8%, 53.0%, 53.2%, 53.4%, 53.6%, 53.8%, 54.0%, 54.2%, 54.4%, 54.6%, 54.8%, 55.0%, 55.2%, 55.4%, 55.6%, 55.8%, 56.0%, 56.2%, 56.4%, 56.6%, 56.8%, 57.0%, 57.2%, 57.4%, 57.6%, 57.8%, 58.0%, 58.2%, 58.4%, 58.6%, 58.8%, 59.0%, 59.2%, 59.4%, 59.6%, 59.8%, 60.0%, 60.2%, 60.4%, 60.6%, 60.8%, 61.0%, 61.2%, 61.4%, 61.6%, 61.8%, 62.0%, 62.2%, 62.4%, 62.6%, 62.8%, 63.0%, 63.2%, 63.4%, 63.6%, 63.8%, 64.0%, 64.2%, 64.4%, 64.6%, 64.8%, 65.0%, 65.2%, 65.4%, 65.6%, 65.8%, 66.0%, 66.2%, 66.4%, 66.6%, 66.8%, 67.0%, 67.2%, 67.4%, 67.6%, 67.8%, 68.0%, 68.2%, 68.4%, 68.6%, 68.8%, 69.0%, 69.2%, 69.4%, 69.6%, 69.8%, 70.0%, 70.2%, 70.4%, 70.6%, 70.8%, 71.0%, 71.2%, 71.4%, 71.6%, 71.8%, 72.0%, 72.2%, 72.4%, 72.6%, 72.8%, 73.0%, 73.2%, 73.4%, 73.6%, 73.8%, 74.0%, 74.2%, 74.4%, 74.6%, 74.8%, 75.0%, 75.2%, 75.4%, 75.6%, 75.8%, 76.0%, 76.2%, 76.4%, 76.6%, 76.8%, 77.0%, 77.2%, 77.4%, 77.6%, 77.8%, 78.0%, 78.2%, 78.4%, 78.6%, 78.8%, 79.0%, 79.2%, 79.4%, 79.6%, 79.8%, 80.0%, 80.2%, 80.4%, 80.6%, 80.8%, 81.0%, 81.2%, 81.4%, 81.6%, 81.8%, 82.0%, 82.2%, 82.4%, 82.6%, 82.8%, 83.0%, 83.2%, 83.4%, 83.6%, 83.8%, 84.0%, 84.2%, 84.4%, 84.6%, 84.8%, 85.0%, 85.2%, 85.4%, 85.6%, 85.8%, 86.0%, 86.2%, 86.4%, 86.6%, 86.8%, 87.0%, 87.2%, 87.4%, 87.6%, 87.8%, 88.0%, 88.2%, 88.4%, 88.6%, 88.8%, 89.0%, 89.2%, 89.4%, 89.6%, 89.8%, 90.0%, 90.2%, 90.4%, 90.6%, 90.8%, 91.0%, 91.2%, 91.4%, 91.6%, 91.8%, 92.0%, 92.2%, 92.4%, 92.6%, 92.8%, 93.0%, 93.2%, 93.4%, 93.6%, 93.8%, 94.0%, 94.2%, 94.4%, 94.6%, 94.8%, 95.0%, 95.2%, 95.4%, 95.6%, 95.8%, 96.0%, 96.2%, 96.4%, 96.6%, 96.8%, 97.0%, 97.2%, 97.4%, 97.6%, 97.8%, 98.0%, 98.2%, 98.4%, 98.6%, 98.8%, 99.0%, 99.2%, 99.4%, 99.6%, 99.8%, or 100.0%.

In some embodiments, the cosmetic skincare compositions and wound healing compositions described herein may include banana stem sap or an extract thereof in an amount by weight (w/w) of, or in an amount by weight (w/w) of about, 5% to 40%, 10% to 30%, 12% to 28%, 16% to 26%, 14% to 24%, or 18% to 22%.

In some embodiments, the cosmetic skincare compositions and wound healing compositions described herein may include banana stem sap or an extract thereof in an amount by weight (w/w) of, or in an amount by weight (w/w) of about, up to 10%, up to 15%, up to 20%, up to 30%, up to 40%, or up to 50%.

The cosmetic skincare composition of the present disclosure includes one or more cosmetically acceptable excipients. The term “cosmetically acceptable” as used herein refers to any pharmaceutically acceptable excipient or additive that is suitable for use in cosmetics, as would be identifiable by skilled persons in view of the present disclosure. The terms “excipient” or “additive” as used herein may refer to any component of the cosmetic skincare composition in addition to the banana stem sap or extract thereof. The cosmetic skincare composition of the present disclosure may include one or more active ingredients. The one or more additives may be one or more active ingredients. The one or more active ingredients includes, but is not limited to, the banana stem sap or extract thereof.

In some embodiments, the cosmetic skincare compositions and wound healing compositions described herein may further include one or more additional active ingredients, in addition to the banana stem sap or extract thereof. In some embodiments, the cosmetic skincare compositions and wound healing compositions described herein may further include one or more additional active ingredients in an amount of, or in an amount of about, 0.01% to 99.9% by weight (w/w). For example, in some embodiments, the cosmetic skincare compositions and wound healing compositions described herein may include one or more additional active ingredients in an amount by weight (w/w) of, or in an amount by weight (w/w) of about, 0.01%, 0.02%, 0.04%, 0.06%, 0.08%, 0.1%, 0.2%, 0.4%, 0.6%, 0.8%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.6%, 8.8%, 9.0%, 9.2%, 9.4%, 9.6%, 9.8%, 10.0%, 10.2%, 10.4%, 10.6%, 10.8%, 11.0%, 11.2%, 11.4%, 11.6%, 11.8%, 12.0%, 12.2%, 12.4%, 12.6%, 12.8%, 13.0%, 13.2%, 13.4%, 13.6%, 13.8%, 14.0%, 14.2%, 14.4%, 14.6%, 14.8%, 15.0%, 15.2%, 15.4%, 15.6%, 15.8%, 16.0%, 16.2%, 16.4%, 16.6%, 16.8%, 17.0%, 17.2%, 17.4%, 17.6%, 17.8%, 18.0%, 18.2%, 18.4%, 18.6%, 18.8%, 19.0%, 19.2%, 19.4%, 19.6%, 19.8%, 20.0%, 20.2%, 20.4%, 20.6%, 20.8%, 21.0%, 21.2%, 21.4%, 21.6%, 21.8%, 22.0%, 22.2%, 22.4%, 22.6%, 22.8%, 23.0%, 23.2%, 23.4%, 23.6%, 23.8%, 24.0%, 24.2%, 24.4%, 24.6%, 24.8%, 25.0%, 25.2%, 25.4%, 25.6%, 25.8%, 26.0%, 26.2%, 26.4%, 26.6%, 26.8%, 27.0%, 27.2%, 27.4%, 27.6%, 27.8%, 28.0%, 28.2%, 28.4%, 28.6%, 28.8%, 29.0%, 29.2%, 29.4%, 29.6%, 29.8%, 30.0%, 30.2%, 30.4%, 30.6%, 30.8%, 31.0%, 31.2%, 31.4%, 31.6%, 31.8%, 32.0%, 32.2%, 32.4%, 32.6%, 32.8%, 33.0%, 33.2%, 33.4%, 33.6%, 33.8%, 34.0%, 34.2%, 34.4%, 34.6%, 34.8%, 35.0%, 35.2%, 35.4%, 35.6%, 35.8%, 36.0%, 36.2%, 36.4%, 36.6%, 36.8%, 37.0%, 37.2%, 37.4%, 37.6%, 37.8%, 38.0%, 38.2%, 38.4%, 38.6%, 38.8%, 39.0%, 39.2%, 39.4%, 39.6%, 39.8%, 40.0%, 40.2%, 40.4%, 40.6%, 40.8%, 41.0%, 41.2%, 41.4%, 41.6%, 41.8%, 42.0%, 42.2%, 42.4%, 42.6%, 42.8%, 43.0%, 43.2%, 43.4%, 43.6%, 43.8%, 44.0%, 44.2%, 44.4%, 44.6%, 44.8%, 45.0%, 45.2%, 45.4%, 45.6%, 45.8%, 46.0%, 46.2%, 46.4%, 46.6%, 46.8%, 47.0%, 47.2%, 47.4%, 47.6%, 47.8%, 48.0%, 48.2%, 48.4%, 48.6%, 48.8%, 49.0%, 49.2%, 49.4%, 49.6%, 49.8%, 50.0%, 50.2%, 50.4%, 50.6%, 50.8%, 51.0%, 51.2%, 51.4%, 51.6%, 51.8%, 52.0%, 52.2%, 52.4%, 52.6%, 52.8%, 53.0%, 53.2%, 53.4%, 53.6%, 53.8%, 54.0%, 54.2%, 54.4%, 54.6%, 54.8%, 55.0%, 55.2%, 55.4%, 55.6%, 55.8%, 56.0%, 56.2%, 56.4%, 56.6%, 56.8%, 57.0%, 57.2%, 57.4%, 57.6%, 57.8%, 58.0%, 58.2%, 58.4%, 58.6%, 58.8%, 59.0%, 59.2%, 59.4%, 59.6%, 59.8%, 60.0%, 60.2%, 60.4%, 60.6%, 60.8%, 61.0%, 61.2%, 61.4%, 61.6%, 61.8%, 62.0%, 62.2%, 62.4%, 62.6%, 62.8%, 63.0%, 63.2%, 63.4%, 63.6%, 63.8%, 64.0%, 64.2%, 64.4%, 64.6%, 64.8%, 65.0%, 65.2%, 65.4%, 65.6%, 65.8%, 66.0%, 66.2%, 66.4%, 66.6%, 66.8%, 67.0%, 67.2%, 67.4%, 67.6%, 67.8%, 68.0%, 68.2%, 68.4%, 68.6%, 68.8%, 69.0%, 69.2%, 69.4%, 69.6%, 69.8%, 70.0%, 70.2%, 70.4%, 70.6%, 70.8%, 71.0%, 71.2%, 71.4%, 71.6%, 71.8%, 72.0%, 72.2%, 72.4%, 72.6%, 72.8%, 73.0%, 73.2%, 73.4%, 73.6%, 73.8%, 74.0%, 74.2%, 74.4%, 74.6%, 74.8%, 75.0%, 75.2%, 75.4%, 75.6%, 75.8%, 76.0%, 76.2%, 76.4%, 76.6%, 76.8%, 77.0%, 77.2%, 77.4%, 77.6%, 77.8%, 78.0%, 78.2%, 78.4%, 78.6%, 78.8%, 79.0%, 79.2%, 79.4%, 79.6%, 79.8%, 80.0%, 80.2%, 80.4%, 80.6%, 80.8%, 81.0%, 81.2%, 81.4%, 81.6%, 81.8%, 82.0%, 82.2%, 82.4%, 82.6%, 82.8%, 83.0%, 83.2%, 83.4%, 83.6%, 83.8%, 84.0%, 84.2%, 84.4%, 84.6%, 84.8%, 85.0%, 85.2%, 85.4%, 85.6%, 85.8%, 86.0%, 86.2%, 86.4%, 86.6%, 86.8%, 87.0%, 87.2%, 87.4%, 87.6%, 87.8%, 88.0%, 88.2%, 88.4%, 88.6%, 88.8%, 89.0%, 89.2%, 89.4%, 89.6%, 89.8%, 90.0%, 90.2%, 90.4%, 90.6%, 90.8%, 91.0%, 91.2%, 91.4%, 91.6%, 91.8%, 92.0%, 92.2%, 92.4%, 92.6%, 92.8%, 93.0%, 93.2%, 93.4%, 93.6%, 93.8%, 94.0%, 94.2%, 94.4%, 94.6%, 94.8%, 95.0%, 95.2%, 95.4%, 95.6%, 95.8%, 96.0%, 96.2%, 96.4%, 96.6%, 96.8%, 97.0%, 97.2%, 97.4%, 97.6%, 97.8%, 98.0%, 98.2%, 98.4%, 98.6%, 98.8%, 99.0%, 99.2%, 99.4%, 99.6%, 99.8%, or 99.9%.

In some embodiments, the cosmetic skincare compositions and wound healing compositions described herein may include one or more additive active ingredients in an amount by weight (w/w) of, or in an amount by weight (w/w) of about, 0.01% to 50%, 0.1% to 40%, 1% to 30%, or 2% to 20%, 5% to 25%, or 10% to 20%.

In some embodiments, the cosmetic skincare compositions and wound healing compositions described herein may include one or more additive active ingredients in an amount by weight (w/w) of, or in an amount by weight (w/w) of about, up to 0.5%, up to 1%, up to 2%, up to 4%, up to 6%, up to 8%, up to 10%, up to 15%, up to 20%, up to 30%, up to 40%, or up to 50%.

In some embodiments, the present disclosure relates to a composition comprising banana stem sap or an extract thereof and one or more pharmaceutically acceptable excipients.

The term “pharmaceutically acceptable” generally means acceptable for administration to a subject, for pharmaceutical purposes, cosmetic purposes, or both, as would be understood by persons of ordinary skill in the art. In some instances, the term “pharmaceutically acceptable” means approved by a regulatory agency of the federal or a state government or listed in the U.S. Pharmacopoeia or other generally recognized pharmacopoeia for use in animals, and more particularly in humans.

In some embodiments, a cosmetically acceptable excipient may not be suitable for including in a composition for applying to a wound. As would be understood by skilled persons, wound care requires particular considerations for safety, healing, and prevention of infection. For example, ingredients commonly found in skincare cosmetics formulations that may not be suitable for applying to a wound may include the following. Fragrances and essential oils: In cosmetics, fragrances and essential oils are often used for pleasant scents; in wound care, fragrances and essential oils may cause irritation in open wounds. Colorants: In cosmetics, colorants may be used for aesthetic purposes; in wound care, colorants may be unnecessary and may cause irritation. Preservatives: In cosmetics, preservatives may be necessary to extend the shelf life of products; in wound care, some preservatives may not be suitable for open wounds or broken skin. Exfoliating Agents: In cosmetics, exfoliating agents may be used for skin exfoliation and renewal; in wound care, exfoliating agents may be too abrasive for healing wounds. Anti-aging ingredients: In cosmetics: ingredients such as retinol or alpha hydroxy acids may be included for anti-aging purposes; in wound care, such anti-aging ingredients may be too harsh for open wounds and may slow down the healing process. Mattefying agents: In cosmetics, mattefying agents may be used to create a matte finish on the skin; in wound care, mattefying agents are not relevant and may not contribute to the healing process. Alcohol-based ingredients: In cosmetics: alcohol-based ingredients may be used for quick drying and as a base for certain formulations; in wound care, alcohol-based ingredients can be too harsh and drying for open wounds. Silicones: in cosmetics, silicones may be used for a smooth texture and to fill in fine lines; in wound care, silicones may not be suitable for healing wounds as they may hinder breathability. Hydroquinone: in cosmetics: hydroquinone may be used for skin lightening; hydroquinone is typically not be applicable in wound care, and may cause irritation.

Fragrances in skincare products can be derived from a variety of natural and synthetic ingredients. Examples of fragrances include, the following, without limitation. Essential Oils: Derived from plants, essential oils are concentrated liquids that capture the fragrance of the plant. Examples include lavender oil, tea tree oil, and chamomile oil. Synthetic Fragrance Oils: These are lab-created fragrance compounds designed to mimic the scents of natural ingredients. They can be more stable and consistent than essential oils. Floral Extracts: Extracts from flowers such as rose, jasmine, and chamomile are commonly used to add a natural floral scent to skincare products. Fruit Extracts: Citrus fruits like orange and lemon, as well as tropical fruits like mango and pineapple, are often used for their pleasant and refreshing scents. Herb Extracts: Extracts from herbs like mint, basil, and thyme can provide a herbal and aromatic fragrance to skincare products. Spice Extracts: Ingredients like cinnamon, clove, and ginger can add warmth and spice to the fragrance profile. Resins: Resins such as frankincense and myrrh are aromatic plant substances that can be used for their distinctive scents. Balsams: Balsamic substances like benzoin and Peru balsam are often used for their sweet and warm fragrance. Wood Extracts: Fragrance from woods like cedarwood, sandalwood, and vetiver can add earthy and woody notes to skincare products. Amber: Amber is a fossilized tree resin and is often used in perfumery for its warm and sweet aroma. Vanilla Extract: Derived from vanilla beans, vanilla extract is commonly used to add a sweet and comforting fragrance to skincare products. Aldehydes: Synthetic compounds that can provide a clean and soapy scent, often used in combination with other fragrances.

Colorants are often added to skincare products to enhance their visual appeal or to differentiate between different formulations. Examples of fragrances include, the following, without limitation. Iron Oxides: These are mineral-based pigments that can provide various shades of red, brown, and yellow. They are commonly used in foundations, blushes, and other cosmetic products. Titanium Dioxide or zinc oxide: titanium dioxide is also used as a white pigment in skincare products, providing opacity and a brightening effect. Ultramarines: These are synthetic pigments that provide vibrant blue, purple, and green shades. They are often used in eyeshadows, mascaras, and other cosmetics. Mica: Mica is a mineral that can be ground into a fine powder to add shimmer and sparkle to skincare and cosmetic products. It comes in various colors. Carmine (CI 75470): This natural colorant is derived from crushed cochineal insects and provides a red to pink hue. It's commonly used in lipsticks, blushes, and other cosmetic products. Annatto (CI 75120): Obtained from the seeds of the achiote tree, annatto is a natural colorant that can range from yellow to orange. It is used in various skincare and cosmetic formulations. Chromium Oxide Greens: These synthetic pigments provide green shades and are used in products like eyeliners and eyeshadows. FD&C Dyes: The FDA-regulated FD&C (Food, Drug, and Cosmetic) dyes, such as FD&C Red No. 40 and FD&C Yellow No. 5, are synthetic colorants used in various skincare and cosmetic products. Chlorophyll: This green pigment found in plants can be used to add a natural green color to skincare products. Turmeric: Known for its anti-inflammatory properties, turmeric is also used as a natural colorant, providing a warm yellow-orange hue. Beetroot Powder: Obtained from dried and ground beetroot, this natural ingredient can add a reddish-pink color to skincare formulations.

Preservatives are useful in skincare products by preventing the growth of bacteria, mold, and other microorganisms that can lead to product spoilage and contamination. Examples of preservatives include, the following, without limitation. Parabens (e.g., Methylparaben, Ethylparaben, Propylparaben): Parabens are a class of synthetic preservatives that have been widely used for their antimicrobial properties. Phenoxyethanol: This is a synthetic preservative with broad-spectrum antimicrobial activity. It's often used in leave-on and rinse-off skincare products. Benzyl Alcohol: Benzyl alcohol is a natural and synthetic compound with antimicrobial properties. It is used as a preservative in various skincare products. Sorbic Acid: Sorbic acid and its salts, such as potassium sorbate, are used as preservatives to inhibit the growth of yeast, mold, and bacteria. Benzoic Acid: Benzoic acid and its salts, such as sodium benzoate, are commonly used as antimicrobial preservatives. They are effective against yeast and bacteria. Chlorphenesin: This synthetic compound is often used as a preservative in cosmetic and skincare formulations to prevent microbial contamination. Ethylhexylglycerin: Ethylhexylglycerin is a synthetic compound that functions as a preservative enhancer. It is often used in combination with other preservatives. Caprylyl Glycol: This synthetic compound is used as a skin-conditioning agent and a preservative booster in skincare formulations. Dehydroacetic Acid: Dehydroacetic acid is a synthetic compound used as a broad-spectrum antimicrobial preservative to prevent the growth of bacteria and fungi. Glyceryl Caprylate: Derived from glycerin and caprylic acid, glyceryl caprylate is used as a co-emulsifier and preservative in skincare products. Sodium Levulinate and Sodium Anisate: These are natural antimicrobial agents derived from anise and levulinic acid. They are often used as part of a preservative system. Gluconolactone: This is a polyhydroxy acid (PHA) with preservative properties. It also provides exfoliation and moisturizing benefits.

Exfoliating agents are used in skincare products to help remove dead skin cells, unclog pores, and promote smoother, brighter skin. There are various types of exfoliating agents, including physical exfoliants and chemical exfoliants. Examples of exfoliants include, the following, without limitation. Physical Exfoliants include the following, without limitation. Jojoba Beads: These are small, spherical beads made from jojoba oil. They provide gentle physical exfoliation without causing microtears in the skin. Apricot Kernel oil or powder: Ground apricot pits are used as a natural exfoliant. Rice Bran Powder: Finely ground rice bran is used for its mild exfoliating properties. It is gentle on the skin and suitable for sensitive skin types. Bamboo Extract: Ground bamboo particles or bamboo powder can be used as a natural exfoliant. It provides a gentle scrubbing action. Pumice: Pumice is a volcanic rock that is ground into a powder and used in some exfoliating products, particularly for rough skin areas like the feet. Almond Meal: Ground almonds can be used as a physical exfoliant. Chemical Exfoliants include the following, without limitation. Alpha Hydroxy Acids (AHAs): Glycolic Acid: Derived from sugar cane, glycolic acid is known for its ability to exfoliate the skin's surface and improve texture. Lactic Acid: This gentle AHA is derived from milk and is suitable for sensitive skin. It helps exfoliate and hydrate simultaneously. Citric Acid: Found in citrus fruits, citric acid is used for exfoliation and is rich in antioxidants. Beta Hydroxy Acids (BHAs): Salicylic Acid: This is a common BHA that is oil-soluble, making it effective for exfoliating within the pores. It's often used in products for acne-prone skin. Enzymes: Papain (from Papaya): Papain is an enzyme found in papaya that helps break down dead skin cells and promote a smoother complexion. Bromelain (from Pineapple): Bromelain, found in pineapple, has exfoliating properties and can help improve skin texture. Polyhydroxy Acids (PHAs): Lactobionic Acid: A gentler alternative to AHAs, lactobionic acid provides exfoliation while also offering antioxidant benefits. Retinoids: Retinol: While primarily known for its anti-aging properties, retinol also promotes exfoliation by encouraging cell turnover.

Many skincare products incorporate various ingredients that are known for their anti-aging properties. These ingredients can help address concerns such as fine lines, wrinkles, loss of elasticity, and uneven skin tone. Examples of anti-aging ingredients include, the following, without limitation. Retinoids (Retinol, Retinoid, Tretinoin): Retinoids are derivatives of vitamin A and are widely recognized for their ability to stimulate collagen production, increase cell turnover, and reduce the appearance of wrinkles. Hyaluronic Acid: A naturally occurring substance in the skin, hyaluronic acid helps maintain hydration by attracting and retaining water. It plumps the skin, reducing the appearance of fine lines and wrinkles. Vitamin C (Ascorbic Acid): Vitamin C is an antioxidant that helps protect the skin from free radical damage. It also promotes collagen synthesis, brightens the skin, and can improve the appearance of skin damage caused by sun exposure. Peptides: Peptides are short chains of amino acids that can signal the skin to produce more collagen. They help improve skin firmness and reduce the appearance of fine lines. Niacinamide (Vitamin B3): Niacinamide has anti-inflammatory properties and helps improve the elasticity of the skin. It can also reduce the appearance of fine lines, hyperpigmentation, and redness. Alpha Hydroxy Acids (AHAs): Glycolic Acid: A type of AHA, glycolic acid exfoliates the skin, promoting cell turnover and improving the appearance of fine lines and uneven skin tone. Lactic Acid: This AHA exfoliates and hydrates, making it suitable for sensitive skin. It can improve the skin's texture and reduce the signs of aging. Beta Hydroxy Acids (BHAs): Salicylic Acid: While commonly known for its acne-fighting properties, salicylic acid can also exfoliate the skin, improving texture and minimizing the appearance of pores. Coenzyme Q10 (Ubiquinone): A powerful antioxidant, CoQ10 helps neutralize free radicals and supports collagen and elastin production. Resveratrol: Found in red grapes and wine, resveratrol is an antioxidant that can protect the skin from environmental damage and promote collagen synthesis. Ceramides: These are lipids that help maintain the skin's barrier function, keeping it hydrated and reducing the appearance of fine lines and wrinkles. Green Tea Extract: Rich in antioxidants, green tea extract helps protect the skin from oxidative stress and may have anti-inflammatory properties. Vitamin E (Tocopherol): An antioxidant that helps protect the skin from free radicals and supports skin barrier function. Matrixyl (Palmitoyl Pentapeptide-4): A peptide that can stimulate collagen synthesis and improve the appearance of fine lines and wrinkles. Centella Asiatica Extract: Provides additional soothing effects and reduces inflammation. Squalane: Mimics natural sebum, improving hydration. Bakuchiol: Offers additional collagen-stimulating benefits. Urea: Increases water retention and provides gentle exfoliation. Sea buckthorn fruit oil: The oil contains high levels of vitamins C and E, which promote collagen production and reduce the appearance of fine lines and wrinkles.

Mattefying agents are ingredients used in skincare products to help control excess oil and shine, providing a matte finish to the skin. Examples of anti-aging ingredients include, the following, without limitation. Silica: Silica is a mineral that absorbs excess oil and has a mattifying effect on the skin. It is often used in powders and primers. Kaolin Clay: Kaolin is a type of clay that absorbs oil and helps control shine. It is commonly found in masks, cleansers, and other skincare products. Bentonite Clay: Similar to kaolin, bentonite clay is another type of clay that can absorb oil and reduce shine on the skin. Salicylic Acid: In addition to its exfoliating properties, salicylic acid can help control oil production, making it suitable for acne-prone and oily skin. Niacinamide (Vitamin B3): Niacinamide has sebum-regulating properties, helping to control oil production and reduce shine on the skin. Mattifying Powders: Various powders, such as rice powder, cornstarch, and talc, are used in skincare products to absorb excess oil and provide a matte finish. Polymeric Spheres: Microspheres made of polymers like polyethylene or polymethylsilsesquioxane can provide a mattifying effect by absorbing oil and diffusing light. Aluminum Starch Octenylsuccinate: This is an aluminum salt of starch that is often used as an absorbent powder in skincare formulations to reduce shine. Dimethicone: While more commonly known as a silicone, dimethicone is also used in some formulations to create a smooth, matte finish on the skin. Zinc Oxide: Apart from its use as a sunscreen ingredient, zinc oxide can also have a mattifying effect on the skin, making it suitable for oily skin types. Witch Hazel Extract: Witch hazel has astringent properties and can help control oil production, contributing to a matte appearance. Astringents: Ingredients like alcohol or witch hazel, when used in moderation, can have a temporary mattifying effect on the skin by reducing oiliness.

Alcohols may be used in skincare products for various purposes, such as solvents, preservatives, or as astringents. Examples of alcohols used in skincare products include, the following, without limitation. Ethanol (Ethyl Alcohol): Ethanol is a common alcohol used as a solvent and preservative in skincare products. It can have a drying effect on the skin and is often used in formulations such as toners and astringents. Isopropyl Alcohol (Isopropanol): Similar to ethanol, isopropyl alcohol is a solvent and antiseptic. It is used in skincare products for its astringent properties. Cetyl Alcohol: Cetyl alcohol is a fatty alcohol that is not drying to the skin. It is used as an emollient and thickening agent in creams and lotions. Stearyl Alcohol: Similar to cetyl alcohol, stearyl alcohol is a fatty alcohol used as an emollient and emulsifier in skincare products. Cetearyl Alcohol: This is a combination of cetyl alcohol and stearyl alcohol. It is a fatty alcohol commonly used as an emollient and emulsifier in creams and lotions. Behenyl Alcohol: Another fatty alcohol, behenyl alcohol, is used as a thickening agent and emollient in skincare formulations. Benzyl Alcohol: Benzyl alcohol can be used as a preservative in skincare products. It has antimicrobial properties and helps prevent the growth of bacteria. Lauric Alcohol: Lauric alcohol is a fatty alcohol used as an emollient and thickening agent in skincare products. Farnesol: Farnesol is a natural alcohol found in some essential oils, and it is used in skincare products for its antimicrobial properties. Glycolic Alcohol (Glycol Alcohol): Glycolic alcohol is derived from glycolic acid and is used as a solvent in skincare formulations.

Silicones are a class of synthetic compounds derived from silicon, oxygen, carbon, and hydrogen. They are often used in skincare products for their various benefits, including providing a smooth texture, enhancing spreadability, and creating a protective barrier on the skin. Examples of silicones used in skincare products include, the following, without limitation. Dimethicone: Dimethicone is a widely used silicone that provides a silky, smooth texture to skincare products. It is often found in moisturizers, primers, and various cosmetic formulations. Dimethicone helps create a barrier on the skin, reducing water loss and enhancing hydration. Cyclopentasiloxane: This is a volatile silicone that evaporates quickly, leaving a smooth, lightweight feel on the skin. Cyclopentasiloxane is often used in serums and other formulations where a light texture is desired. Cyclomethicone: Similar to cyclopentasiloxane, cyclomethicone is a volatile silicone with a light, non-greasy feel. It is commonly used in skincare and hair care products to improve the product's texture. Dimethiconol: This is a silicone derivative of dimethicone, providing similar benefits, such as enhanced texture, slip, and skin conditioning. Dimethiconol is often used in leave-on skincare products. Polysiloxane: Polysiloxane is a group of polymers containing silicon and oxygen. It is used in skincare products for its film-forming and conditioning properties. Trimethicone: Trimethicone is a silicone that contributes to the smooth texture of skincare products. Phenyl Trimethicone: This silicone is known for its conditioning properties and is used to improve the feel and texture of skincare and hair care products. Amodimethicone: Amodimethicone is a type of silicone used in hair and skincare products for its conditioning and detangling properties. It helps improve the combability of hair and provides a smooth feel to the skin. Silicone Crosspolymers: These are silicone polymers that contribute to the spreadability and texture of skincare products. They can enhance the sensory experience and provide a soft, velvety finish. PEG/PPG Dimethicone: This is a silicone copolymer often used as an emollient and skin-conditioning agent in skincare formulations.

Hydroquinone is a chemical compound that is used in skincare products for its skin-lightening properties. It works by inhibiting the production of melanin, the pigment responsible for skin color, and is commonly used to treat hyperpigmentation, including dark spots, age spots, and melasma. Examples of hydroquinones used in skincare products include, the following, without limitation. Hydroquinone: The most common and well-known hydroquinone is simply named hydroquinone. It is available in various concentrations in over-the-counter (OTC) and prescription skincare products. Arbutin: Arbutin is a naturally occurring derivative of hydroquinone found in plants like bearberry, cranberry, and pear. It is often used in skincare products for its skin-brightening effects. There are two forms of arbutin: alpha-arbutin and beta-arbutin. Kojic Acid: Kojic acid is a byproduct of the fermentation process of malting rice for sake. It is sometimes used in skincare products for its skin-lightening properties, as it inhibits melanin production. Mequinol: Mequinol is a derivative of hydroquinone that is used in some prescription skincare products. It works similarly to hydroquinone by inhibiting melanin production. Ellagic Acid: Found in certain fruits like strawberries and pomegranates, ellagic acid is sometimes used in skincare products for its antioxidant and skin-brightening properties.

For example, and not by limitation, in some embodiments, the compositions described herein may include glycerin, jojoba oil, apricot kernel oil, sea buckthorn fruit oil, camellia seed oil, squalene, sodium ascorbyl phosphate, tocopherol, coenzyme Q10, hyaluronic acid, allantoin, vitamin C, ferulic acid, ceramides, retinoids, peptides, bakuchiol, urea, green tea extract, panthenol, madecassoside, niacinamide, Centella Asiatica extract, palmitoyl tripeptide-1, resveratrol, Chlorella Vulgaris extract, sea kelp bioferment, spirulina extract, Laminaria Digitata extract, Arnica extract, aloe vera extract, Fucus Vesiculosus extract, copper peptides, or any combinations thereof.

In some embodiments, the cosmetic skincare composition may be formulated to be rubbed, poured, sprinkled, or sprayed on, introduced into, or otherwise applied to the skin of a subject.

In some embodiments, the cosmetic skincare composition may be formulated for topical administration to the skin.

In some embodiments, a topical cosmetic skincare formulation of the present disclosure may be, or may comprise, a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.

As would be understood by skilled persons, creams are emulsions of oil and water that are typically semi-solid. They are easy to spread and are often used for moisturizing. Ointments are semi-solid preparations with a higher concentration of oil than creams. They provide a barrier on the skin and are useful for dry or damaged skin. Lotions are a liquid preparation that is often a mixture of oil and water. Lotions are lighter than creams and are easily spread on the skin. Gels are semisolid systems containing a gelling agent to give them a jelly-like consistency. Pastes are thick preparations with a high concentration of solids. Liniments are liquid or semi-liquid preparations intended for rubbing on the skin. Sprays are liquid formulations that are sprayed onto the skin. Patches include but are not limited to transdermal patches. Foams are aerosol formulations that dispense a foam. Serums are typically more concentrated formulations that may provide faster penetration to the skin. Powders are typically dry, finely ground substances that may used alone or may be mixed with water or other vehicles before application. Mousses are light, foamy formulations. Balms are semi-solid preparations that often contain waxes. Liposomes are spherical vesicles with a lipid bilayer, which may be used to improve the delivery of active ingredients. Hydrogels are water-based gels. A microemulsion is a thermodynamically stable dispersion of oil and water, stabilized by an amphiphilic surfactant. The term “micro” refers to the small droplet size of the dispersed phase (oil) in the range of approximately 10 to 100 nanometers. A nanoemulsion is a type of emulsion in which tiny droplets of one immiscible liquid (such as oil) are dispersed in another immiscible liquid (such as water) with the help of surfactants and/or cosurfactants. The distinguishing feature of a nanoemulsion is the extremely small size of its droplets, typically in the nanometer range (20 to 200 nanometers). In some embodiments, the cosmetic skincare composition may be formulated for disposition into a bandage. In some embodiments, the cosmetic skincare composition may be disposed in a bandage.

The banana stem sap compounds described herein can also be administered in sustained release forms or from sustained release drug delivery systems. Sustained release materials are known in the art and representative sustained release materials are described in the literature references cited herein.

For example, topical cream formulations are typically an emulsion semisolid dosage form that is used for skin external application. Typically, topical cream formulations contain more than 20% water and volatiles and/or less than 50% of hydrocarbons, waxes, or polyethylene glycols as a vehicle for external skin application. In a topical cream formulation, ingredients are typically dissolved or dispersed in either a water-in-oil (W/O) emulsion or an oil-in-water (O/W) emulsion.

Excipients used in a topical cream formulation may include thickeners or gelling agents, emulsifying agents, preservatives, antioxidants, and buffer agents. Manufacture of a topical cream formulation typically includes excipient dissolution, phase mixing, introduction of active ingredients, and homogenization of the product mixture. The topical formulations of the present disclosure may be prepared using any suitable method known in the art.

In topical cream formulations, oily compounds are typically used as active ingredient carriers. Oily compounds commonly used in cream formulations include saturated and unsaturated fatty acids/fatty acid esters, hydrocarbons, and polyols. Thickeners such as sclerotium gum, methylcellulose and paraffin may be used to increase the physical stability of the water-in-oil (W/O) emulsion or an oil-in-water (O/W) emulsion in the cream. Emulsifying agents can reduce the interfacial tension between the two phases, thus retarding phase separation. Ionic surfactants are typically used in oil-in-water emulsions, whereas nonionic surfactants may be used in both oil-in-water and water-in-oil formulations. Oils and fats used in topical cream formulations are susceptible to oxidation by atmospheric oxygen or microorganism action. The stability against oxidation can be enhanced by the introduction of preservatives or antioxidants. By buffering potential pH change, buffer agents can provide chemical stability.

The components described herein for a topical formulation of the present disclosure are merely representative. Other materials as well as processing techniques and the like are known in the art, for example as described in Remington's Pharmaceutical Sciences, 17th edition, 1985, Mack Publishing Company, Easton, Pa.; Remington: The Science and Practice of Pharmacy (22nd ed.)., Troy, D. B., & Beringer, P. (Eds.) (2012), Lippincott Williams & Wilkins; Pharmaceutical Dosage Forms and Drug Delivery Systems (11th ed.)., Ansel, H. C., Allen, L. V. Jr., & Popovich, N. G. (Eds.) (2016), Lippincott Williams & Wilkins; Dermatological and Transdermal Formulations, Walters, K. A., & Hadgraft, J. (Eds.) (2002), CRC Press; Topical Drug Delivery Formulations, Osborne, D. W., & Amann, A. H. (Eds.) (2002), Marcel Dekker; and Cosmetic Dermatology: Principles and Practice (2nd ed.)., Baumann, L. (Ed.) (2009) McGraw-Hill Education, all of which are incorporated herein by reference.

Excipients and additives of the present disclosure may include, without limitation, various carriers, emulsifiers, chelation agents, stabilizers, enhancers, preservatives, pH adjusters, texture and sensory modifiers, fragrances, antioxidants, anticoagulants, and botanical extracts, among others.

In some embodiments, the cosmetic skincare compositions and wound healing compositions described herein may include one or more excipients in an amount by weight (w/w) of, or in an amount by weight (w/w) of about 0.01%, 0.02%, 0.04%, 0.06%, 0.08%, 0.1%, 0.2%, 0.4%, 0.6%, 0.8%, 1.0%, 1.2%, 1.4%, 1.6%, 1.8%, 2.0%, 2.2%, 2.4%, 2.6%, 2.8%, 3.0%, 3.2%, 3.4%, 3.6%, 3.8%, 4.0%, 4.2%, 4.4%, 4.6%, 4.8%, 5.0%, 5.2%, 5.4%, 5.6%, 5.8%, 6.0%, 6.2%, 6.4%, 6.6%, 6.8%, 7.0%, 7.2%, 7.4%, 7.6%, 7.8%, 8.0%, 8.2%, 8.4%, 8.6%, 8.8%, 9.0%, 9.2%, 9.4%, 9.6%, 9.8%, 10.0%, 10.2%, 10.4%, 10.6%, 10.8%, 11.0%, 11.2%, 11.4%, 11.6%, 11.8%, 12.0%, 12.2%, 12.4%, 12.6%, 12.8%, 13.0%, 13.2%, 13.4%, 13.6%, 13.8%, 14.0%, 14.2%, 14.4%, 14.6%, 14.8%, 15.0%, 15.2%, 15.4%, 15.6%, 15.8%, 16.0%, 16.2%, 16.4%, 16.6%, 16.8%, 17.0%, 17.2%, 17.4%, 17.6%, 17.8%, 18.0%, 18.2%, 18.4%, 18.6%, 18.8%, 19.0%, 19.2%, 19.4%, 19.6%, 19.8%, 20.0%, 20.2%, 20.4%, 20.6%, 20.8%, 21.0%, 21.2%, 21.4%, 21.6%, 21.8%, 22.0%, 22.2%, 22.4%, 22.6%, 22.8%, 23.0%, 23.2%, 23.4%, 23.6%, 23.8%, 24.0%, 24.2%, 24.4%, 24.6%, 24.8%, 25.0%, 25.2%, 25.4%, 25.6%, 25.8%, 26.0%, 26.2%, 26.4%, 26.6%, 26.8%, 27.0%, 27.2%, 27.4%, 27.6%, 27.8%, 28.0%, 28.2%, 28.4%, 28.6%, 28.8%, 29.0%, 29.2%, 29.4%, 29.6%, 29.8%, 30.0%, 30.2%, 30.4%, 30.6%, 30.8%, 31.0%, 31.2%, 31.4%, 31.6%, 31.8%, 32.0%, 32.2%, 32.4%, 32.6%, 32.8%, 33.0%, 33.2%, 33.4%, 33.6%, 33.8%, 34.0%, 34.2%, 34.4%, 34.6%, 34.8%, 35.0%, 35.2%, 35.4%, 35.6%, 35.8%, 36.0%, 36.2%, 36.4%, 36.6%, 36.8%, 37.0%, 37.2%, 37.4%, 37.6%, 37.8%, 38.0%, 38.2%, 38.4%, 38.6%, 38.8%, 39.0%, 39.2%, 39.4%, 39.6%, 39.8%, 40.0%, 40.2%, 40.4%, 40.6%, 40.8%, 41.0%, 41.2%, 41.4%, 41.6%, 41.8%, 42.0%, 42.2%, 42.4%, 42.6%, 42.8%, 43.0%, 43.2%, 43.4%, 43.6%, 43.8%, 44.0%, 44.2%, 44.4%, 44.6%, 44.8%, 45.0%, 45.2%, 45.4%, 45.6%, 45.8%, 46.0%, 46.2%, 46.4%, 46.6%, 46.8%, 47.0%, 47.2%, 47.4%, 47.6%, 47.8%, 48.0%, 48.2%, 48.4%, 48.6%, 48.8%, 49.0%, 49.2%, 49.4%, 49.6%, 49.8%, 50.0%, 50.2%, 50.4%, 50.6%, 50.8%, 51.0%, 51.2%, 51.4%, 51.6%, 51.8%, 52.0%, 52.2%, 52.4%, 52.6%, 52.8%, 53.0%, 53.2%, 53.4%, 53.6%, 53.8%, 54.0%, 54.2%, 54.4%, 54.6%, 54.8%, 55.0%, 55.2%, 55.4%, 55.6%, 55.8%, 56.0%, 56.2%, 56.4%, 56.6%, 56.8%, 57.0%, 57.2%, 57.4%, 57.6%, 57.8%, 58.0%, 58.2%, 58.4%, 58.6%, 58.8%, 59.0%, 59.2%, 59.4%, 59.6%, 59.8%, 60.0%, 60.2%, 60.4%, 60.6%, 60.8%, 61.0%, 61.2%, 61.4%, 61.6%, 61.8%, 62.0%, 62.2%, 62.4%, 62.6%, 62.8%, 63.0%, 63.2%, 63.4%, 63.6%, 63.8%, 64.0%, 64.2%, 64.4%, 64.6%, 64.8%, 65.0%, 65.2%, 65.4%, 65.6%, 65.8%, 66.0%, 66.2%, 66.4%, 66.6%, 66.8%, 67.0%, 67.2%, 67.4%, 67.6%, 67.8%, 68.0%, 68.2%, 68.4%, 68.6%, 68.8%, 69.0%, 69.2%, 69.4%, 69.6%, 69.8%, 70.0%, 70.2%, 70.4%, 70.6%, 70.8%, 71.0%, 71.2%, 71.4%, 71.6%, 71.8%, 72.0%, 72.2%, 72.4%, 72.6%, 72.8%, 73.0%, 73.2%, 73.4%, 73.6%, 73.8%, 74.0%, 74.2%, 74.4%, 74.6%, 74.8%, 75.0%, 75.2%, 75.4%, 75.6%, 75.8%, 76.0%, 76.2%, 76.4%, 76.6%, 76.8%, 77.0%, 77.2%, 77.4%, 77.6%, 77.8%, 78.0%, 78.2%, 78.4%, 78.6%, 78.8%, 79.0%, 79.2%, 79.4%, 79.6%, 79.8%, 80.0%, 80.2%, 80.4%, 80.6%, 80.8%, 81.0%, 81.2%, 81.4%, 81.6%, 81.8%, 82.0%, 82.2%, 82.4%, 82.6%, 82.8%, 83.0%, 83.2%, 83.4%, 83.6%, 83.8%, 84.0%, 84.2%, 84.4%, 84.6%, 84.8%, 85.0%, 85.2%, 85.4%, 85.6%, 85.8%, 86.0%, 86.2%, 86.4%, 86.6%, 86.8%, 87.0%, 87.2%, 87.4%, 87.6%, 87.8%, 88.0%, 88.2%, 88.4%, 88.6%, 88.8%, 89.0%, 89.2%, 89.4%, 89.6%, 89.8%, 90.0%, 90.2%, 90.4%, 90.6%, 90.8%, 91.0%, 91.2%, 91.4%, 91.6%, 91.8%, 92.0%, 92.2%, 92.4%, 92.6%, 92.8%, 93.0%, 93.2%, 93.4%, 93.6%, 93.8%, 94.0%, 94.2%, 94.4%, 94.6%, 94.8%, 95.0%, 95.2%, 95.4%, 95.6%, 95.8%, 96.0%, 96.2%, 96.4%, 96.6%, 96.8%, 97.0%, 97.2%, 97.4%, 97.6%, 97.8%, 98.0%, 98.2%, 98.4%, 98.6%, 98.8%, 99.0%, 99.2%, 99.4%, 99.6%, 99.8%, or 99.9%.

In some embodiments, the cosmetic skincare compositions and wound healing compositions described herein may include one or more excipients in an amount by weight (w/w) of, or in an amount by weight (w/w) of about, 0.01% to 50%, 0.1% to 40%, 1% to 30%, or 2% to 20%, 5% to 25%, or 10% to 20%.

In some embodiments, the cosmetic skincare compositions and wound healing compositions described herein may include one or more excipients in an amount by weight (w/w) of, or in an amount by weight (w/w) of about, up to 0.5%, up to 1%, up to 2%, up to 4%, up to 6%, up to 8%, up to 10%, up to 15%, up to 20%, up to 30%, up to 40%, or up to 50%.

For example, and not by limitation, in some embodiments, the compositions described herein may include aloe vera, sodium phytate, sclerotium gum, mango butter, Olivem® 1000 (Hallstar Italia S.r.l. (Italy), stearic acid, rosehip oil, phenoxyethanol, ethylhexylglycerin, or any combinations thereof.

The following non-limiting examples illustrate representative excipients that may be used in accordance with the present disclosure.

Example carriers include, without limitation, aloe vera gel, natural oils such as jojoba oil, argan oil, rosehip oil, coconut oil, avocado oil, sweet almond oil, shea butter, mango butter, vegetable glycerin, and squalene, among others. Example emulsifiers and stabilizers include, without limitation, lecithin, cetearyl alcohol, xanthan gum, Olivem® 1000 (Hallstar Italia S.r.l.; Italy), glyceryl stearate, stearic acid, and guar gum, among others.

Example chelation agents include, without limitation, sodium phytate.

Example sources of squalene include, without limitation, camellia seed oil.

Example enhancers include, without limitation, hyaluronic acid, vitamin E (tocopherols), vitamin C (ascorbic acid), peptides, ceramides, niacinamide, retinol, alpha hydroxy acids (e.g., glycolic acid and lactic acid), and omega-3 and omega-6 fatty acids, among others.

Example preservatives include, without limitation, grapefruit seed extract, rosemary extract, sorbic acid, phenonip, tea tree oil, thyme extract, and benzyl alcohol, among others.

Example pH adjusters include, without limitation, citric acid, lactic acid, and potassium hydroxide, among others.

Example texture and sensory modifiers include, without limitation, beeswax or carnauba wax, silk amino acids, arrowroot powder, cocoa butter, bentonite clay, and colloidal oatmeal, among others.

Example fragrances include, without limitation, lavender oil, chamomile extract, neroli oil, rose oil, ylang ylang oil, and geranium oil, among others.

Example antioxidants include, without limitation, green tea extract, coenzyme Q10, beta-carotene, ferulic acid, sodium ascorbyl phosphate, and astaxanthin, among others.

Example botanical extracts include, without limitation, calendula extract, green tea extract, chamomile extract, willow bark extract, and licorice root extract, among others.

Example anticoagulants include witch hazel, among others.

In some embodiments, the cosmetic skincare composition of the present disclosure may comprise an amount of the banana stem sap or extract thereof effective to increase fibroblast proliferation in the skin, increase Platelet-Derived Growth Factor BB (PDGF-BB) in the skin, increase collagen production in the skin, or any combinations thereof, following administration of the composition to skin of a subject. Fibroblasts are a type of connective tissue cell that plays a crucial role in maintaining the structural framework of tissues and organs in the body. These cells are found in various connective tissues, including the dermis of the skin, tendons, ligaments, and the matrix of cartilage and bone. Fibroblasts are responsible for synthesizing and secreting extracellular matrix components, such as collagen, elastin, and glycoproteins, which provide strength, elasticity, and support to the surrounding tissues. Fibroblasts are the primary cells responsible for producing the extracellular matrix (ECM) components. The ECM forms a scaffold that supports cells and tissues, contributing to their structural integrity. Collagen is a major component of the ECM, providing tensile strength to tissues. Fibroblasts synthesize and secrete various types of collagen, depending on the tissue they inhabit. Fibroblasts play a crucial role in the process of wound healing and tissue repair. They migrate to the site of injury and produce ECM components to facilitate the closure and healing of wounds. Fibroblasts are involved in cell signaling and communication within the extracellular matrix. They respond to various signals from other cells, growth factors, and cytokines, influencing tissue development, repair, and remodeling. Fibroblasts contribute to the maintenance of tissue structure and homeostasis by continuously synthesizing and remodeling the extracellular matrix. Dysregulation of fibroblast activity can contribute to various pathological conditions, including fibrosis, where excessive ECM deposition occurs, leading to tissue scarring and loss of function.

Without intending to be limited by theory, fibroblast proliferation, activation, or both, aids in formation and remodeling of the extracellular matrix, thereby facilitating rapid tissue regeneration. Without intending to be limited by theory, PDGF-BB production enhances the recruitment and proliferation of fibroblasts and assists in granulation tissue formation. Without intending to be limited by theory, increased activity of fibroblasts leads to a significant upsurge in collagen production.

In some embodiments, the cosmetic skincare composition and methods of use thereof described herein are effective to increase fibroblast proliferation, activation, or both, by up to 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more.

In some embodiments, the compositions and methods of use thereof described herein are effective to produce gene expression changes in the skin that are associated with one or more of the beneficial effects described herein. For example, see Example 1.

Methods for detecting increased fibroblast proliferation, increased PDGF-BB, and increased collagen production, may include, without limitation, detection in cultured skin cells in vitro, or in biopsies of skin cells from a human or animal subject, or in an animal model (e.g., a mouse model).

Detecting increased fibroblast proliferation can be done through various experimental and laboratory techniques known in the art, including the following, without limitation. Direct cell counting: Direct cell counting using a hemocytometer or automated cell counter can provide a simple measure of cell proliferation. Increased cell numbers may indicate heightened fibroblast proliferation. Bromodeoxyuridine (BrdU) Incorporation Assay: BrdU is a thymidine analog that is incorporated into newly synthesized DNA during cell division. Detection of BrdU incorporation using immunostaining or enzyme-linked immunosorbent assay (ELISA) can indicate actively proliferating cells. 3-(4,5-Dimethylthiazol-2-yl)-2,5-diphenyltetrazolium Bromide (MTT) Assay: The MTT assay measures the reduction of MTT to formazan by metabolically active cells. This can be used as an indicator of cell viability and proliferation. Ki-67 Staining: Ki-67 is a protein expressed in proliferating cells during the cell cycle. Immunostaining for Ki-67 can be used to identify and quantify actively dividing cells, including fibroblasts. Flow Cytometry: Flow cytometry allows for the analysis of cell cycle distribution and can be used to identify and quantify proliferating cell populations based on DNA content and cell cycle markers. Real-time Polymerase Chain Reaction (RT-PCR): Quantitative PCR can be employed to measure the expression levels of genes associated with cell proliferation, such as those involved in the cell cycle (e.g., cyclins, CDKs). Western Blotting: Protein expression levels of markers associated with cell proliferation, such as PCNA (proliferating cell nuclear antigen) or cyclins, can be assessed through western blotting. Incorporation of Radioactive Nucleotides: Incorporation of radioactive nucleotides, such as thymidine, into DNA during replication can be used to measure cell proliferation. Live-Cell Imaging: Using time-lapse microscopy, changes in cell morphology, division, and migration can be observed.

Platelet-Derived Growth Factor-BB (PDGF-BB) is a member of the PDGF family of growth factors, which play a crucial role in cell proliferation, growth, and differentiation. PDGF-BB is a dimeric protein composed of two B chains linked by disulfide bonds. It is one of the isoforms of PDGF, with other isoforms including PDGF-AA, PDGF-AB, PDGF-CC, and PDGF-DD. PDGF-BB is composed of two B chains, which are polypeptide chains linked together by disulfide bonds. This dimeric structure is biologically active and binds to its receptors to initiate cellular responses. PDGF-BB primarily binds to two types of receptors, known as PDGFR-α and PDGFR-β. These receptors are tyrosine kinase receptors located on the cell surface. Upon binding of PDGF-BB, these receptors undergo autophosphorylation and activate downstream signaling pathways involved in cell growth and survival. PDGF-BB is involved in various biological processes, including the regulation of cell proliferation, migration, and survival. It plays a crucial role in embryonic development, tissue repair, and angiogenesis (the formation of new blood vessels). Additionally, PDGF-BB has been implicated in diseases such as cancer and fibrotic disorders. PDGF-BB acts on a variety of cell types, including fibroblasts, smooth muscle cells, and glial cells. Its effects are particularly important in the context of wound healing and tissue repair, where it stimulates the proliferation and migration of cells involved in these processes. Dysregulation of PDGF signaling is associated with excessive tissue scarring.

Detecting increased levels of PDGF-BB can be done through various laboratory techniques, including the following, without limitation. Enzyme-Linked Immunosorbent Assay (ELISA): ELISA is a widely used technique for the quantitative measurement of proteins, including growth factors like PDGF-BB. In this assay, a specific antibody against PDGF-BB is immobilized on a solid surface. The sample containing PDGF-BB is added, and if PDGF-BB is present, it binds to the antibody. A second enzyme-linked antibody is then added, and a colorimetric reaction is used to quantify the amount of PDGF-BB present. Western Blotting: Western blotting can be used to detect and semi-quantify proteins in a sample. It involves separating proteins by gel electrophoresis, transferring them to a membrane, and then probing the membrane with a specific antibody against PDGF-BB. The intensity of the signal can give an indication of the relative amount of PDGF-BB. Immunohistochemistry (IHC): IHC is a technique used to visualize the presence and distribution of proteins in tissue samples. Tissue sections are treated with antibodies specific to PDGF-BB, and the binding is visualized using a detection system. This method provides information about the localization of PDGF-BB within tissues. PCR (Polymerase Chain Reaction): PCR can be used to measure the expression levels of PDGF-BB at the mRNA level. This method is particularly useful for detecting changes in gene expression. However, it does not directly measure the protein levels. Biological Assays: Functional assays can be employed to measure the biological activity of PDGF-BB. These assays may involve using cell cultures and assessing the proliferative or migratory responses of cells to PDGF-BB. While these assays are more complex, they provide information about the functional significance of the detected PDGF-BB. Mass Spectrometry: Mass spectrometry can be used for quantitative analysis of proteins, including PDGF-BB. This method involves ionizing and separating proteins based on their mass-to-charge ratio, allowing for accurate measurement of protein levels.

Collagen is a fibrous protein that serves as a crucial structural component in the connective tissues of animals, including humans. It is the most abundant protein in the human body and is found in various tissues such as skin, bones, tendons, ligaments, cartilage, and blood vessels. Collagen provides strength, structure, and elasticity to these tissues. The basic structural unit of collagen is a triple helix, which is composed of three polypeptide chains known as alpha chains. These chains are tightly coiled around each other, forming a stable and robust structure. The specific arrangement of amino acids in collagen contributes to its unique properties, such as tensile strength and resistance to stretching. There are multiple types of collagen in the human body, each with its own function and distribution in different tissues. For example, type I collagen is prevalent in the skin, bones, and tendons, providing strength and support. Type II collagen is found in cartilage, contributing to its flexibility and resilience. Type III collagen is often associated with the structure of blood vessels and internal organs. Collagen is synthesized by fibroblasts, and its production can be influenced by various factors, including genetics, nutrition, and overall health. As people age, collagen production tends to decrease, leading to changes in the skin, joints, and other connective tissues.

Detecting increased levels of collagen can be done through various laboratory techniques, including the following, without limitation. Biopsy or Tissue Sampling: In some cases, a biopsy or tissue sample may be taken from the target area to analyze collagen content. Histological staining techniques, such as Masson's trichrome or Picrosirius Red, can highlight collagen fibers in tissue samples under a microscope. Imaging Techniques: Non-invasive imaging techniques, such as ultrasound, magnetic resonance imaging (MRI), or computed tomography (CT) scans, can sometimes provide information about tissue composition, including collagen levels. Blood Biomarkers: Blood tests may be used to measure specific biomarkers associated with collagen metabolism. For example, markers like procollagen peptides can indicate collagen synthesis. Skin Biophysical Measurements: In dermatology, techniques like cutometer measurements or elastography can be used to assess skin elasticity and stiffness, which can be influenced by collagen content. Collagen-Specific Imaging Agents: Molecular imaging techniques, using specific agents that target collagen, can provide information about collagen distribution and concentration. Clinical Observations: Clinical assessment by healthcare professionals can sometimes provide indirect evidence of increased collagen production. For example, changes in skin texture, joint mobility, or the appearance of scars may suggest altered collagen levels. Serum Biomarkers: Some markers in the blood, like collagen metabolites or specific enzymes involved in collagen synthesis, can be indicative of increased collagen production.

In some embodiments, the compositions and methods of use thereof described herein are effective to up-regulate production of collagen, including but not limited to type I collagen, type III collagen, type IV collagen, or any combinations thereof. For example, see Example 2.

In some embodiments, the compositions and methods of use thereof described herein are effective to increase skin cell numbers, including but not limited to fibroblast numbers. In some embodiments, the compositions and methods of use thereof described herein are effective to up-regulate skin cell proliferation, including but not limited to fibroblast proliferation. For example, see Example 2.

Collagenases and elastases are enzymes that break down collagens, elastin and other proteinaceous components of the extracellular matrix (ECM) in the skin and other tissues. In the skin, the activity of both enzymes is induced by UVA/UVB irradiation and other inflammatory processes and may contribute to the process of photoaging.

In some embodiments, the compositions and methods of use thereof described herein are effective to inhibit the activity of collagenases, elastases, or both collagenases and elastases. For example, see Example 3. Of note, Example 3 shows that not all banana stem sap may be effective to inhibit the activity of collagenases, elastases, or both collagenases and elastases. The selection of a banana stem sap from a particular member of the Musa genus that is effective to inhibit the activity of collagenases, elastases, or both collagenases and elastases, may be unpredictable. Surprisingly, Example 3 shows that sap from Musa Balbisiana provided mild to moderate inhibition of collagenase (MP) enzymatic activity and elastase activity, whereas sap from a hybrid of Musa acuminata and Musa balbisiana did not show inhibition of collagenase activity or inhibition of elastase activity.

In some embodiments, the cosmetic skincare composition of the present disclosure may comprise an amount of the banana stem sap or extract thereof effective to reduce wrinkles in the skin, reduce fine lines in the skin, reduce skin laxity (e.g. sagging skin), improve skin texture, improve skin tone, reduce visibility of scars, reduce stretch marks, reduce age spots, reduce hyperpigmentation, reduce skin roughness, reduce uneven pigmentation, reduce dullness, increase skin hydration, increase skin elasticity, increase skin firmness, reduce enlarged skin pore size, reduce acne, reduce acne scars, reduce skin damage caused by sun exposure (for example without limitation, skin damage associated with exposure to UVA radiation, UVB radiation, or a combination thereof; also known as “photoaging”), reduce skin damage caused by exposure to pollution, reduce under-eye skin circles, reduce under-eye skin bags, reduce skin puffiness, increase skin radiance, increase skin glow, reduce skin irritation, reduce skin rashes (e.g. diaper rashes, among others), reduce skin blemishes, promote skin tightening, reduce eczema, or any combinations thereof, following administration of the composition to skin of a subject.

In some embodiments, the present disclosure relates a method of treating or preventing a cosmetic skin condition in a subject. The method comprises administering to the subject's skin an effective amount of banana stem sap or an extract thereof, or an effective amount of a skincare composition described herein comprising banana stem sap or an extract thereof and one or more cosmetically acceptable excipients.

In some embodiments, the subject may be a vertebrate animal. In some embodiments, the subject may be a mammal. In some embodiments, the subject may be a primate. In some embodiments, the subject may be a human.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

“Treating” or “treatment” of a cosmetic skin condition refers, in some embodiments, to ameliorating the cosmetic skin condition (e.g., arresting or reducing the development of the cosmetic skin condition or at least one of the symptoms thereof). In some embodiments “treating” or “treatment” refers to ameliorating at least one physical parameter, which may not be discernible by the subject. In some embodiments, “treating” or “treatment” refers to modulating the cosmetic skin condition, either physically, (e.g., stabilization of a discernible symptom), physiologically, (e.g., stabilization of a physical parameter), or both.

“Preventing” or “prevention” refers to a reduction in risk of acquiring a cosmetic skin condition (e.g., causing at least one of the symptoms of the cosmetic skin condition not to develop in a subject not yet exposed to or predisposed or susceptible to the cosmetic skin condition, and not yet experiencing or displaying symptoms of the cosmetic skin condition).

A “cosmetic skin condition” includes, without limitation, wrinkles in the skin, fine lines in the skin, skin laxity (e.g. sagging skin), undesirable skin texture, uneven skin texture, uneven skin tone, scars, stretch marks, age spots, hyperpigmentation, skin roughness, uneven pigmentation, skin dullness, skin dryness, insufficient skin elasticity, insufficient skin firmness, enlarged skin pore size, acne, acne scars, skin damage caused by sun exposure (for example without limitation, skin damage associated with exposure to UVA radiation, UVB radiation, or a combination thereof; also known as “photoaging”), skin damage caused by exposure to pollution, under-eye skin circles, under-eye skin bags, skin puffiness, insufficient skin radiance, insufficient skin glow, or any combinations thereof.

Accordingly, in some embodiments, the method of treating or preventing a cosmetic skin condition in a subject is effective to reduce wrinkles in the skin, reduce fine lines in the skin, reduce skin laxity, improve skin texture, improve skin tone, reduce visibility of scars, reduce stretch marks, reduce age spots, reduce hyperpigmentation, reduce skin roughness, reduce uneven pigmentation, reduce dullness, increase skin hydration, increase skin elasticity, increase skin firmness, reduce enlarged skin pore size, reduce acne, reduce acne scars, reduce skin damage caused by sun exposure (for example without limitation, skin damage associated with exposure to UVA radiation, UVB radiation, or a combination thereof; also known as “photoaging”), reduce skin damage caused by exposure to pollution, reduce under-eye skin circles, reduce under-eye skin bags, reduce skin puffiness, increase skin radiance, increase skin glow, or any combinations thereof. In some embodiments, the method of treating or preventing a cosmetic skin condition in a subject is effective to prevent or treat effects of aging on the skin of the subject.

In some embodiments, the subject's skin to which the cosmetic skincare composition is administered does not comprise a wound. In some embodiments, no wound is present on the area of skin to which the cosmetic skincare composition is applied. In some embodiments, the term “aging” refers to natural non-pathological processes. In some embodiments, “aging” refers to changes in the skin that are not associated with a disease. In some embodiments, “aging” refers to natural processes of developments, such as puberty, e.g., in producing acne. In some embodiments, aging refers to processes in absence of acute or chronic injury. In some embodiments, a wound may be a result of, for example and without limitation, an acute injury, laceration, impact, bruise, burn, lesion, surgical incision, skin graft, or any combinations thereof. In some embodiments, a wound is a result of a chronic condition or disease such as but not limited to a diabetic ulcer.

In some embodiments, the method of treating or preventing a cosmetic skin condition in a subject is effective to increase fibroblast proliferation in the skin, increased PDGF-BB in the skin, increased collagen production in the skin, or any combinations thereof, following administration of the composition to skin of a subject.

In some embodiments, the administration may be one, two, three, four, five, or six times per day. In some embodiments, the administration may be constant or substantially constant, such as from a patch, for a period of time. In some embodiments, the period of time may be up to 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or up to 2 days, 3 days, 4 days, 5 days, 6 days, 7 days, or up to 2 weeks, 3 weeks, 4 weeks, or up to 2 months, 3 months, 4 months, 5 months, 6 months, or up to a year or more.

In some embodiments, the present disclosure relates to a wound healing composition comprising banana stem sap or an extract thereof and one or more pharmaceutically acceptable excipients.

As used herein, the term “wound healing composition” relates to a composition intended to be applied to the skin of a subject for purposes of treatment of a wound.

In some embodiments, the wound healing composition comprises banana stem sap or an extract thereof from any species of the genus Musa described herein.

In some embodiments, the wound healing composition may be formulated to be rubbed, poured, sprinkled, or sprayed on, introduced into, or otherwise applied to the skin of a subject.

In some embodiments, the wound healing composition may be formulated for topical administration to the skin.

In some embodiments, a topical wound healing formulation of the present disclosure may be, or may comprise, a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof. In some embodiments, the topical wound healing composition may be formulated for disposition into a bandage. In some embodiments, the topical wound healing formulation may be disposed in a bandage.

In some embodiments, the wound healing composition of the present disclosure may comprise an amount of the banana stem sap or extract thereof effective to increase fibroblast proliferation in the skin, increased PDGF-BB in the skin, increased collagen production in the skin, or any combinations thereof, following administration of the composition to skin of a subject.

In some embodiments, the wound healing composition and methods of use thereof described herein are effective to increase fibroblast proliferation, activation, or both, by up to 10%, 20%, 30%, 40%, 50%, 60%, 70%, 80%, 90%, 100%, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or more.

In some embodiments, the wound healing composition and methods of use thereof described herein are effective to produce gene expression changes in the skin that are associated with one or more of the beneficial effects described herein.

In some embodiments, the wound healing composition of the present disclosure may comprise an amount of the banana stem sap or extract thereof effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems (such as HIV/AIDS or undergoing chemotherapy), improve in the healing of skin and tissue damage caused by radiation treatments, reduce scar formation, or any combination thereof, following administration of the composition to a wound in skin of a subject.

In some embodiments, the wound healing composition of the present disclosure may comprise an amount of the banana stem sap or extract thereof effective to prevent or decrease a wound infection such as a bacterial infection or a fungal infection, among others. For example, without limitation, in some embodiments, the infection may be an E. coli infection, a Bacillus megaterium infection, a Pseudomonas aeruginosa infection, a Staphylococcus aureus infection, or any combinations thereof, among others.

In some embodiments, the wound healing composition of the present disclosure may comprise an amount of the banana stem sap or extract thereof effective to decrease wound bleeding time by up to 10%, 20%, 30%, 40%, 50%, 60%, 70%, 80%, 90%, 95%, 99%, or more. In some embodiments, the wound healing composition of the present disclosure may comprise an amount of the banana stem sap or extract thereof effective to decrease wound closure time by up to 10%, 20%, 30%, 40%, 50%, 60%, 70%, 80%, 90%, 95%, 99%, or more.

In some embodiments, the wound healing composition of the present disclosure may comprise an amount of the banana stem sap or extract thereof effective to decrease wound healing time by up to 10%, 20%, 30%, 40%, 50%, 60%, 70%, 80%, 90%, 95%, 99%, or more.

In some embodiments, the wound healing composition of the present disclosure may comprise an amount of the banana stem sap or extract thereof effective to decrease clotting time by up to 10%, 20%, 30%, 40%, 50%, 60%, 70%, 80%, 90%, 95%, 99%, or more.

In some embodiments, the wound may be or may include, without limitation, an acute wound, a chronic wound, a cut, an abrasion, a surgical incision, a skin graft, a traumatic wound, a hypertrophic scar, an ulcer, such as a diabetic ulcer or a venous ulcer, a pressure sore, a surgical wound, a post-operative wound, a burn wound, skin or tissue damage caused by radiation treatments, or any combination thereof.

In some embodiments, the present disclosure relates a method of treating a subject for a wound. The method comprises administering to the subject's skin an effective amount of the wound healing composition described herein comprising banana stem sap or an extract thereof and one or more pharmaceutically acceptable excipients.

In some embodiments, the subject may be a vertebrate animal. In some embodiments, the subject may be a mammal. In some embodiments, the subject may be a primate. In some embodiments, the subject may be a human.

“Treating” or “treatment” of a wound refers, in some embodiments, to aiding in healing of the wound, ameliorating the wound (e.g., arresting or reducing worsening of the wound or at least one of the symptoms related to the wound). In some embodiments “treating” or “treatment” refers to ameliorating at least one physical parameter, which may not be discernible by the subject. In some embodiments, “treating” or “treatment” refers to modulating the wound, either physically, (e.g., stabilization of a discernible symptom), physiologically, (e.g., stabilization of a physical parameter), or both.

Accordingly, in some embodiments, the method of treating a wound in the skin of a subject is effective to decrease wound healing time, decrease wound closure time, promote skin graft integration, reduce scar tissue formation, reduce scar visibility, reduce or prevent wound infection, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems (such as HIV/AIDS or undergoing chemotherapy), improve in the healing of skin and tissue damage caused by radiation treatments, reduce scar formation, or any combination thereof, following administration of the composition to a wound in skin of a subject.

In some embodiments, the method of treating a wound in the skin of a subject is effective to increase fibroblast proliferation in the skin, increased PDGF-BB in the skin, increased collagen production in the skin, or any combinations thereof, following administration of the composition to skin of a subject.

In some embodiments, a compositions and methods described herein may be combined with other compositions and methods, including known skincare active ingredients, compositions and methods, either in the same composition, or administered separately. In some embodiments, the compositions and methods of the present disclosure may act synergistically with the other active ingredients, compositions and methods, so that the resulting compositions and/or methods demonstrate improved effectiveness.

In some embodiments, the compositions and methods described herein may be useful for applications as skincare products, wound care products, or both, for example as consumer skincare products and/or wound care products, such as over-the counter products, or products prescribed by a healthcare professional. In some embodiments, the compositions and methods described herein may be useful for applications in emergency scenarios, such as in first aid kits, wound care for home use, for clinical use, for military use, for first responder uses, and the like.

In some embodiments, the compositions and methods described herein are appealing to users of skincare products and wound care products, as comprising natural ingredients, such as those derived from banana stem sap. There has been a long felt need in the market for such products and methods as described herein for skincare, wound care, or both, that has been challenging to meet. In some embodiments, the compositions and methods described herein solve problems of providing solutions to such long felt need.

In some embodiments, the compositions described herein are non-toxic, show improved tolerability, improved stability, improved efficacy, or any combinations thereof, compared to previously existing compositions and products. In some embodiments, the compositions described herein show improved tolerability for use on sensitive skin.

In some embodiments, the compositions described herein may use organic ingredients, such as organic banana stem sap. In some embodiments, the banana stem sap is obtained from banana waste products, such as banana stem waste byproducts from banana fruit harvesting.

In some embodiments, the cosmetic skincare compositions and wound healing compositions described herein comprising banana stem sap, e.g. Balbisiana sap or extracts thereof, optionally further comprising one or more additives and/or excipients described herein, may show beneficial effects, synergistic activity, and improved efficacy for use in cosmetic skincare compositions, wound healing compositions, or both.

For example, and without intending to be limited by theory, the Examples disclosed herein provide evidence that compositions described herein comprising banana stem sap, e.g., Balbisiana sap or extracts thereof, optionally further comprising one or more additives and/or excipients described herein have beneficial effects, synergies, and improved efficacy on endpoints such as skin health related gene expression changes, collagen production, collagenase and elastase inhibition, and cell proliferation, for example as shown experimentally in normal human neonatal dermal fibroblasts. For example, and not by way of limitation, in some embodiments, example compositions comprising Balbisiana Sap (e.g., 1%), Balbisiana Sap (e.g., 1%)+Allantoin (e.g., 0.5%), and Balbisiana Sap (e.g., 1%)+Hyaluronic Acid (e.g., 0.5%) show beneficial effects, synergies, and improved efficacy for use in cosmetic skincare compositions, wound healing compositions, or both.

In some embodiments, such example compositions show significant modulation in collagen synthesis, anti-inflammatory pathways, antioxidant response, barrier repair, hydration, metalloproteinase, and elastase inhibition. In some embodiments, various combinations of the example compositions comprising Balbisiana sap or extracts thereof, optionally further comprising one or more additives and/or excipients show distinct profiles with synergistic activity when combined with one or more additional skincare active ingredients.

In some embodiments, the cosmetic skincare compositions and wound healing compositions described herein may include banana step sap or an extract thereof (for example, without limitation, from Musa Balbisiana) and optionally one or more excipients, and optionally one or more additive active ingredients.

In some embodiments, the banana stem sap or extract thereof may be present in the composition in an amount (w/w) of, or of about, 1% to 50%, 5% to 40%, 10% to 30%, or 15% to 25%.

In some embodiments, the banana stem sap or extract thereof may be present in the composition in an amount (w/w) of, or of about, up to 1%, up to 5%, up to 10%, up to 20%, up to 30%, up to 40%, or up to 50%.

In some embodiments, the one or more excipients and/or more additive active ingredients may be present in the composition in an amount (w/w) of, or of about, up to 0.5%, up to 1%, up to 2%, up to 5%, or up to 10%.

In some embodiments, the additive active ingredients may include Allantoin, Hyaluronic Acid, or both Allantoin and Hyaluronic Acid. In some embodiments, the Allantoin or Hyaluronic Acid may be present in the composition in an amount (w/w) of, or of about, up to 0.5%, up to 1%, up to 2%, up to 5%, or up to 10%.

In some embodiments, compositions comprising Balbisiana sap or extracts thereof, optionally further comprising one or more additives and/or excipients show improved anti-inflammatory and immune modulation effects that are beneficial for cosmetic skincare compositions, wound healing compositions, or both. For example, and not by way of limitation, the Examples described herein show the following beneficial effects:

For example, Balbisiana Sap (1%) shows potent downregulation of IL17C (−2.01 fold), PSORS1C2 (−10.36 fold), and IL22RA1 (−2.13 fold), indicating a broad anti-inflammatory effect.

IL-17C is a pro-inflammatory cytokine that can contribute to inflammation, including epithelial inflammation in psoriasis and atopic eczema. PSORS1C2, or Psoriasis Susceptibility 1 Candidate 2, is a gene that is associated with psoriasis. Symptoms of PSORS1C2 inflammation include red patches of skin with silvery-white scales that can burn or itch. Plaques and scales may appear on any part of the body, although they are commonly found on the elbows, knees, and scalp. IL22RA1 is a receptor for the cytokine interleukin 22 (IL-22), which is involved in inflammation.

For example, Balbisiana Sap+Allantoin (0.5%) further enhances anti-inflammatory effects, with greater downregulation of IL17C (−3.18 fold) and CCL24 (−2.10 fold).

CCL24, or CC motif chemokine ligand 24, is a chemokine that regulates inflammation and fibrosis. CCL24 is constitutively expressed by dermal fibroblasts and is involved in type 2 immune reactions that contribute to skin lesions.

For example, Balbisiana Sap+Hyaluronic Acid (0.5%) maintains significant downregulation of IL17C (−3.47 fold). Example quantified benefits include the following: IL17C downregulation by 101-153%: Contributes to reduced inflammation, beneficial for managing sensitive skin, redness, and conditions like psoriasis. CCL24 downregulation by 104-110%: Reduces immune cell trafficking, beneficial for calming inflammatory responses. In some embodiments, example synergistic excipients and/or active ingredients may include, without limitation: Niacinamide (up to 130% increase in anti-inflammatory activity): Further reduces redness and supports barrier repair. Centella Asiatica Extract: Provides additional soothing effects and reduces inflammation.

In some embodiments, compositions comprising Balbisiana sap or extracts thereof, optionally further comprising one or more additives and/or excipients show improved antioxidant support and oxidative stress response effects that are beneficial for cosmetic skincare compositions, wound healing compositions, or both. For example, and not by way of limitation, the Examples described herein show the following beneficial effects: Balbisiana Sap (1%) reduces HMOX1 (−3.44 fold) and CAT (−2.60 fold), indicating a strong antioxidant effect by lowering internal oxidative stress. Balbisiana Sap+Allantoin (0.5%) maintains a moderate reduction in oxidative stress (HMOX1 down −1.86 fold). Balbisiana Sap+Hyaluronic Acid (0.5%) shows upregulation of HMOX1 (+1.71 fold), indicating potential enhancement of internal antioxidant capacity. Example quantified benefits include the following: HMOX1 downregulation up to 344% suggests reduced oxidative stress, contributing to visible anti-aging effects and skin resilience. In some embodiments, example synergistic excipients and/or active ingredients may include, without limitation: Vitamin C: Increases total antioxidant capacity when combined with Balbisiana Sap. Ferulic Acid: Further stabilizes and amplifies antioxidant effects.

In some embodiments, compositions comprising Balbisiana sap or extracts thereof, optionally further comprising one or more additives and/or excipients show improved barrier function and lipid metabolism effects that are beneficial for cosmetic skincare compositions, wound healing compositions, or both. For example, and not by way of limitation, the Examples described herein show the following beneficial effects: Balbisiana Sap (1%) shows downregulation of lipid-related genes like ABCG4 (−3.86 fold) and SLC27A4 (−2.72 fold), suggesting enhanced barrier integrity. Balbisiana Sap+Allantoin (0.5%) upregulates CA12 (+1.59 fold), promoting hydration and lipid retention. Balbisiana Sap+Hyaluronic Acid (0.5%) shows increased expression of ATP2B4 (+97%), indicating improved hydration mechanisms. Example quantified benefits include the following: CA12 upregulation by 59%: Supports lipid retention and barrier function. ATP2B4 upregulation by 97%: Enhances hydration and moisture balance. In some embodiments, example synergistic excipients and/or active ingredients may include, without limitation: Ceramides: Reinforce lipid retention and barrier integrity. Squalane: Mimics natural sebum, improving hydration.

In some embodiments, compositions comprising Balbisiana sap or extracts thereof, optionally further comprising one or more additives and/or excipients show improved tissue remodeling, collagen synthesis, and structural support effects that are beneficial for cosmetic skincare compositions, wound healing compositions, or both. For example, and not by way of limitation, the Examples described herein show the following beneficial effects: Collagen Production: Type I Collagen: Balbisiana Sap 0.5% increases type I collagen production by 136% of control (p=0.000). Balbisiana Sap 1.6% increases type I collagen by 103% (p=0.675). Type III Collagen: Balbisiana Sap 0.5% shows a 257% increase in type III collagen (p=0.004). Balbisiana Sap 1.6% results in a 191% increase (p=0.020). Type IV Collagen: Balbisiana Sap 0.5% upregulates type IV collagen by 267% of control (p=0.000). Balbisiana Sap 1.6% leads to a 286% increase (p=0.000). Example quantified benefits include the following: Significant increases in type I (up to 136%), type III (up to 257%), and type IV collagen (up to 286%), supporting skin firmness, elasticity, and structural integrity. In some embodiments, example synergistic excipients and/or active ingredients may include, without limitation: Retinoids: Enhance collagen synthesis and complement Balbisiana Sap's tissue remodeling effects. Peptides: Further support collagen production and improve extracellular matrix integrity. Bakuchiol: Offers additional collagen-stimulating benefits.

In some embodiments, compositions comprising Balbisiana sap or extracts thereof, optionally further comprising one or more additives and/or excipients show improved hydration and aquaporin modulation effects that are beneficial for cosmetic skincare compositions, wound healing compositions, or both. For example, and not by way of limitation, the Examples described herein show the following beneficial effects: Balbisiana Sap (1%) upregulates AQP5 (+2.91 fold), contributing to moisture retention. Balbisiana Sap+Hyaluronic Acid (0.5%) significantly upregulates ATP2B4 (+97%), enhancing cellular hydration. Example quantified benefits include the following: AQP5 upregulation by 291%: Contributes to improved water transport and hydration. ATP2B4 upregulation by 97%: Enhances cellular hydration and moisture balance. In some embodiments, example synergistic excipients and/or active ingredients may include, without limitation: Glycerin: Enhances skin's moisture-binding capacity. Urea: Increases water retention and provides gentle exfoliation.

In some embodiments, compositions comprising Balbisiana sap or extracts thereof, optionally further comprising one or more additives and/or excipients show improved metalloproteinase and elastase inhibition effects that are beneficial for cosmetic skincare compositions, wound healing compositions, or both. For example, and not by way of limitation, the Examples described herein show the following beneficial effects: Metalloproteinase inhibition: Balbisiana Sap 10% reduces collagenase activity by 51%, with a moderate inhibitory effect at 5% (72% of control) and 1% (89% of control). Elastase Inhibition: Balbisiana Sap 0.5% shows the strongest elastase inhibition, reducing activity to 71% of control, suggesting moderate suppression of elastase, a key enzyme in skin aging. Example quantified benefits include the following: Collagenase inhibition up to 51%: Reduces collagen breakdown, supporting anti-aging and tissue preservation. Elastase inhibition up to 29%: Decreases elastin degradation, enhancing skin elasticity. In some embodiments, example synergistic excipients and/or active ingredients may include, without limitation: Allantoin: Enhances wound healing while also contributing to elastase inhibition. Green Tea Extract: Provides additional inhibition of metalloproteinase activity, complementing Balbisiana Sap's effects.

In some embodiments, compositions comprising Balbisiana sap or extracts thereof, optionally further comprising one or more additives and/or excipients may show improved wound healing and repair effects that are beneficial for cosmetic skincare compositions, wound healing compositions, or both. For example, and not by way of limitation, the Examples described herein show the following beneficial effects: Balbisiana Sap (1%) upregulates BDKRB1 (+1.82 fold), promoting wound healing. Balbisiana Sap+Allantoin (0.5%) significantly amplifies wound healing with BDKRB1 upregulation (+3.74 fold). Example quantified benefits include the following: BDKRB1 upregulation by 82-374%: Accelerates wound healing and tissue regeneration. In some embodiments, example synergistic excipients and/or active ingredients may include, without limitation: Panthenol (Pro-Vitamin B5): Enhances wound healing and hydration. Madecassoside: Supports collagen synthesis and wound repair.

Surprisingly, the Examples described herein show some notable differences in beneficial effects among various combinations of Balbisiana sap or extracts thereof, optionally further comprising one or more additives and/or excipients, including for example, and not by way of limitation, the following: Balbisiana Sap+Allantoin (0.5%) demonstrates strong overall beneficial effects, with significant increases in collagen types I (136%), III (257%), IV (267%), as well as strong upregulation of wound healing markers and collagenase inhibition. In contrast, Balbisiana Sap+Hyaluronic Acid (0.5%) shows focused hydration improvements, with ATP2B4 upregulation (97%), enhancing moisture retention and cellular hydration. Further in contrast, Balbisiana Sap (1%) alone offers balanced anti-inflammatory, antioxidant, and structural support effects, making it versatile for anti-aging and hydration-focused formulations.

In some embodiments, the following active ingredients may have synergistic beneficial effects when combined with Musa Balbisiana Sap or extracts thereof, in cosmetic skincare compositions, wound healing compositions, or both. Synergistic beneficial effects may be observed on one or more parameters, such as collagen synthesis, hydration, barrier repair, and anti-inflammatory action. In some embodiments, the following active ingredients may show synergistic activity when combined with Musa Balbisiana Sap or extracts thereof, in cosmetic skincare compositions, wound healing compositions, or both:

Niacinamide: Niacinamide stimulates collagen synthesis, reduces inflammation, and strengthens the skin barrier. Synergy: In the collagen study (see e.g., Example 2), niacinamide increased type I collagen production by up to 130%, complementing banana sap's upregulation of type I, III, and IV collagen by 136%, 257%, and 267% respectively. Benefits: Enhanced skin firmness, reduced redness, improved even tone, and barrier repair.

Bakuchiol: Bakuchiol is a plant-derived retinol alternative known to stimulate collagen production and provide antioxidant protection. Synergy: Bakuchiol's ability to enhance collagen synthesis may act synergistically with banana sap's upregulation of collagen genes and reduction of metalloproteinase activity by up to 51%. Benefits: Improved skin elasticity, wrinkle reduction, and antioxidant support with reduced irritation.

Centella Asiatica Extract (Gotu Kola): Contains active compounds like asiaticoside and madecassoside, which promote wound healing, collagen synthesis, and reduce inflammation. Synergy: Centella Asiatica complements banana sap's upregulation of BDKRB1 by up to 374%, supporting faster wound healing and improved collagen synthesis. Benefits: Enhanced healing, stronger anti-inflammatory effects, and increased skin firmness.

Palmitoyl Tripeptide-1 (Matrixyl 3000): Stimulates collagen synthesis and improves extracellular matrix support. Synergy: Banana sap's effects on types I, III, and IV collagen can be further enhanced by peptides like Palmitoyl Tripeptide-1, known for increasing skin density and firmness. Benefits: Increased skin elasticity, reduction in fine lines, and improved skin structure.

Green Tea Extract (EGCGN) inhibits metalloproteinases, reduces inflammation, and has antioxidant properties. Synergy: Green tea extract aligns with banana sap's 51% reduction in collagenase activity and overall anti-inflammatory effects, providing complementary protection against collagen degradation. Benefits: Reduced collagen breakdown, improved skin elasticity, and protection against oxidative stress.

Vitamin C (Ascorbic Acid, Tetrahexyldecyl Ascorbate) increases collagen synthesis and provides strong antioxidant protection. Synergy: Vitamin C supports banana sap's upregulation of type I and III collagen by 136% and 257%, respectively, and reduces oxidative stress, aligning with banana sap's antioxidant gene modulation. Benefits: Improved skin firmness, reduced oxidative damage, and enhanced collagen production.

Hyaluronic Acid (Low and High Molecular Weight) boosts moisture retention and improves hydration at multiple skin layers. Synergy: Banana sap's ATP2B4 upregulation (+97%) aligns with hyaluronic acid's ability to enhance water transport and retention, leading to deeper and longer-lasting hydration. Benefits: Enhanced skin hydration, plumping, and moisture balance, leading to improved skin texture.

Panthenol (Pro-Vitamin B5) supports wound healing, hydration, and barrier repair. Synergy: Panthenol's wound-healing properties complement banana sap's upregulation of BDKRB1 (+374%), promoting faster recovery and improved hydration. Benefits: Increased moisture retention, accelerated healing, and reduced skin irritation.

Retinoids (Retinol, Retinaldehyde) stimulate collagen synthesis, improve cell turnover, and enhance skin firmness. Synergy: Retinoids' collagen-stimulating effects align with banana sap's upregulation of collagen production, leading to enhanced anti-aging results. Benefits: Improved collagen production, reduction of fine lines, and smoother skin texture, with potential mitigation of retinoid irritation by banana sap's anti-inflammatory effects.

Resveratrol acts as an antioxidant and inhibits metalloproteinases. Synergy: Resveratrol complements banana sap's collagenase inhibition (up to 51%) and reduction in pro-inflammatory gene expression. Benefits: Enhanced antioxidant protection, reduced collagen degradation, and improved elasticity.

Ceramides reinforce skin barrier function and improve lipid retention. Synergy: Banana sap's gene modulation of lipid metabolism (e.g., ABCG4 down −3.86 fold) aligns with ceramides' barrier-enhancing properties. Benefits: Increased barrier strength, better moisture retention, and reduced transepidermal water loss (TEWL).

Squalane mimics natural skin sebum, providing deep hydration and barrier protection. Synergy: Squalane supports banana sap's barrier-enhancing and lipid-retention effects, improving overall moisture balance. Benefits: Improved hydration, softer skin texture, and enhanced barrier repair.

Chlorella Vulgaris Extract, an algae extract, supports collagen synthesis and offers antioxidant protection. Synergy: Chlorella's collagen-enhancing properties align with banana sap's upregulation of collagen types I, III, and IV. Benefits: Increased skin firmness, reduced wrinkles, and protection against environmental stressors.

Sea Kelp Bioferment is rich in minerals and polysaccharides, sea kelp bioferment supports barrier repair, moisture retention, and anti-inflammatory effects. Synergy: Sea kelp's hydrating and anti-inflammatory effects complement banana sap's gene modulation related to hydration and reduced inflammation. Benefits: Enhanced hydration, reduced irritation, and improved skin resilience.

Spirulina Extract contains amino acids and minerals that boost collagen production and provide antioxidant protection. Synergy: Spirulina's amino acid profile supports banana sap's upregulation of collagen genes and reduction in metalloproteinase activity. Benefits: Improved skin elasticity, increased hydration, and enhanced protection against oxidative stress.

Laminaria Digitata (Brown Algae) Extract is rich in alginates, it helps retain moisture and has anti-inflammatory properties. Synergy: Laminaria complements banana sap's barrier and hydration-enhancing effects, promoting moisture retention and reduced irritation. Benefits: Increased hydration, reduced inflammation, and improved skin texture.

Arnica Extract offers strong anti-inflammatory and wound-healing benefits. Synergy: Arnica's wound-healing properties align with banana sap's upregulation of BDKRB1 (+374%) and reduction in pro-inflammatory genes. Benefits: Faster recovery from skin damage, reduced redness, and improved overall skin resilience.

Aloe Vera Extract is known for its soothing, hydrating, and wound-healing properties. Synergy: Aloe vera's hydration and wound-healing effects complement banana sap's collagen upregulation and anti-inflammatory action. Benefits: Enhanced soothing, deeper hydration, and faster healing of minor wounds or irritations.

Fucus Vesiculosus (Bladderwrack) Extract is a seaweed extract that is rich in polysaccharides and minerals that support collagen synthesis, moisture retention, and anti-inflammatory effects. Synergy: Bladderwrack's collagen-boosting properties align with banana sap's ability to upregulate multiple collagen types, while its hydrating effects support banana sap's AQP5 upregulation (+2.91 fold). Benefits: Improved elasticity, enhanced moisture retention, and reduced inflammation.

Copper Peptides stimulate collagen production, enhance wound healing, and reduce inflammation. Synergy: Copper peptides enhance banana sap's effects on collagen types I, III, and IV, supporting overall anti-aging and wound repair benefits. Benefits: Accelerated collagen production, improved healing, and greater skin resilience.

The above additional active ingredients may provide synergistic effects with banana stem sap, e.g., Balbisiana Sap. Each active is selected based on its ability to enhance specific aspects of banana sap's bioactivity, e.g., in collagen synthesis, hydration, barrier repair, and inflammation reduction. The combinations can target diverse skin concerns, providing superior outcomes by maximizing each ingredient's potential through complementary mechanisms. Quantifiable effects, where demonstrated, further validate these synergies, making these example combinations suitable for high-efficacy skincare formulations.

### EXAMPLES

The present examples are provided for illustrative purposes only. They are not intended to and should not be interpreted to encompass the full breadth of the invention.

### Example 1. Effect of Two Test Materials on the Expression of a Panel of Genes of Interest in UVB-Irradiated Human HaCaT Keratinocytes

This Example describes an experiment on the effects of the test materials listed in Table 1 on the expression of a panel of genes in UVB-irradiated human HaCaT keratinocytes, serving as a model of photoaging.

### Materials and Methods

TABLE 1
Test materials assayed:

| Test material | Lot # | Form | Identifier |
|---|---|---|---|
| Raw Banana Sap | 230924-W-T1 | Brown | B.E. |
| | | liquid | |
| Raw Banana Sap | 230924-BDF-T1 | Brown | B.E. Past. |
| Pasteurized | | liquid | |

Test materials. Test materials were stored at 4° C. All dilutions were made in sterile distilled water the day of the experiment. Samples of test materials were added at ×20 final dose, in a 25 μl volume to 475 μl cell culture medium per well.

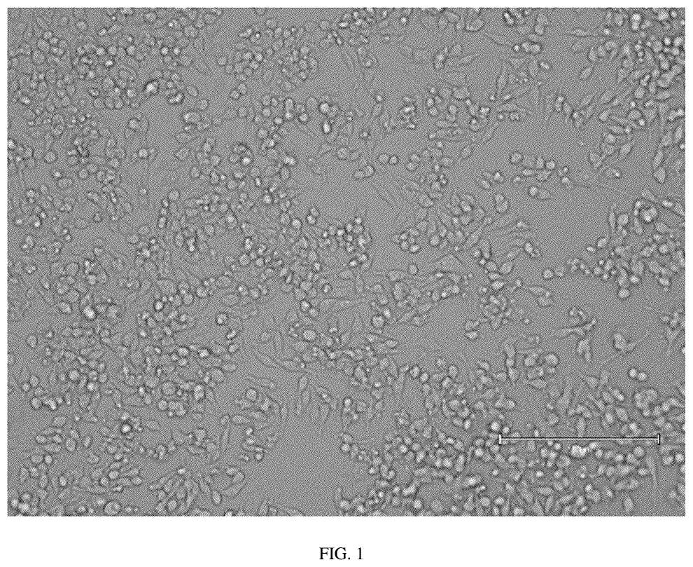

Experimental Procedure. HaCaT keratinocytes (AddexBio (San Diego, CA)) as shown in FIG. 1 were plated in a 24 well plate in DMEM/10% FBS (Sigma, St. Louis)/Pen+Strep+Fungizone solution (Lonza, Switzerland). The following day both test materials were added to confluent late subcultures in triplicates at the concentration of 0.5% (v: v). That concentration was decided based on the previous experiment aimed at determining the non-cytotoxic range of concentrations for B.E and B.E. Past., in which no cytotoxicity was detected up to the highest tested dose (10%). Sterile distilled water was the negative control.

Keratinocytes were allowed to incubate for a three-hour period with test materials, afterward they were placed in PBS (phosphate buffered saline) and were exposed to 30 mJ/cm² (equivalent to 1 minimal erythema dose, or MED) UVB (302 nm), in the presence of test materials, using Hoefer UVTM-19-115V (Holliston, MA) transilluminator (except one triplicate of non-exposed controls). Cells were then placed back in the culture medium in the presence of the test substances and were allowed to incubate overnight.

At the end of the incubation period HaCaTs were rinsed and RNA was extracted and purified with RNeasy Mini Plus Kit (Qiagen, German) using QiaCube Connect robotic station (Qiagen). Purified total RNA was assessed at 260 nm and 280 nm with NanoDrop Lite (ThermoFisher Scientific, Waltham, MA). cDNA was prepared using High-Capacity RNA-to-cDNA™ Kit (Thermo Fisher Scientific) and the expression of the genes of interest was measured by real-time quantitative PCR using C1000 Touch System with CFX384™ optical module, 100 nM/well of PCR primers from Realtimeprimers (Elkins Park, PA) and Azura View GreenFast qPCR Blue Mix LR (Azura Genomics, Raynham, MA). Efficiency ΔΔCt method was used for quantification of results, after the normalization of gene expression to PPIA and RPS18 (housekeeping genes). Ct (cycle threshold) is defined as the number of replication cycles required for the fluorescent signal to exceed the background level. Ct levels are inversely proportional to the amount of target nucleic acid in the sample (i.e., the lower the Ct level the greater the amount of target nucleic acid in the sample).

Genes were considered differentially expressed if the p value, as determined by the two-tailed t-test, was ≤0.1, the modulation was ≥1.7 and the expression level was <33 cycles required for detection.

STRING database (https://string-db.org) was used to visualize the predicted protein-protein interaction networks within differentially expressed gene sets.

### Results and Discussion

### Comparison Between UVB-Irradiated and Non-Irradiated HaCaT Keratinocytes.

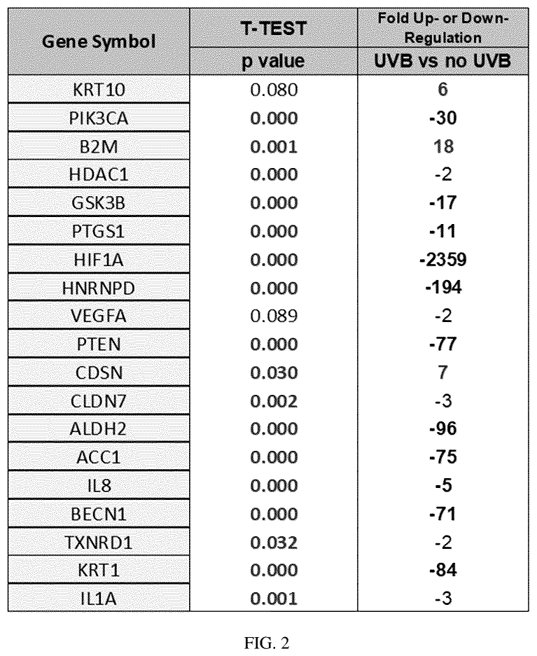

FIG. 2 shows the list of genes whose expression has been significantly modulated by UVB irradiation.

The directionality of this modulation is in agreement with published reports. See for example, an article by Umar S A, Shahid N H, Nazir L A, Tanveer M A, Divya G, Archoo S, Raghu S R, Tasduq S A entitled “Pharmacological Activation of Autophagy Restores Cellular Homeostasis in Ultraviolet-(B)-Induced Skin Photodamage” in Front Oncol. 2021 Aug. 2; 11:726066. doi: 10.3389/fonc.2021.726066. PMID: 34408986; PMCID: PMC8366585.). The results may indicate an immunosuppressive response, and technically validate the experiment. See, e.g., an article by Stephen E. Ullrich entitled “Mechanisms underlying UV-induced immune suppression, Mutation Research/Fundamental and Molecular Mechanisms of Mutagenesis, Volume 571, Issues 1-2, 2005, Pages 185-205, ISSN 0027-5107, https://doi.org/10.1016/j.mrfmmm.2004.06.059.

### B.E.

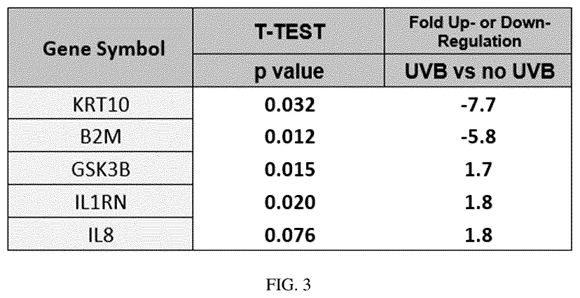

FIG. 3 shows the list of genes whose expression has been significantly modulated by UVB irradiation of B.E.-treated HaCaT keratinocytes, vs. the water-treated ones. Surprisingly, the pattern of that modulation was focused on the reversion of the UVB-induced changes (compare FIG. 2 and FIG. 3). In another words, the addition of B.E. at least partially protected HaCaT keratinocytes from the UVB-induced alteration of gene expression. Furthermore, the activation of the IL1RN expression was consistent with an anti-inflammatory activity of that test material.

In conclusion, the bioactivity profile of B.E. identified in this Example consisted in a partial prevention of UVB irradiation from the disruption of the cellular homeostasis. Therefore, B.E. may play a protective role in skin photoaging.

### Example 2. Effect of Raw Saps of Musa Balbisiana and Dwarf Blue Field Banana on Collagen I, Collagen III, Collagen IV and Cell Numbers in Human Fibroblast Cell Populations

This Example describes an experiment on the compounds listed in Table 2 on the output of a series of extracellular matrix components by neonatal human dermal fibroblasts.

### Materials and Methods

TABLE 2
Test materials assayed:

| Test material | Lot # | Form | Identifier |
|---|---|---|---|
| Raw sap of Musa | 20240212-BAL- | Brown | MB-2024 |
| Balbisiana | RAW CENTRI | opaque | |
| | | liquids w/ | |
| | | particles | |
| Raw Sap of Dwarf | 20240212-DBF- | Brown | DBF-2024 |
| Blue Field Banana | RAW CENTRI | opaque | |
| (Musa Acuminata × | | liquids w/ | |
| Balbisiana) | | particles | |

Test materials. Test materials (Table 2) were stored at +4° C. and were diluted in distilled sterile water immediately before testing.

Cells & experimental procedures. Normal dermal human neonatal dermal fibroblasts (ScienCell; Oceanside, CA, cat #2310, passage 5-6) were seeded in DMEM with 10% serum at medium density to achieve late exponential growth stage overnight (Day 0).

The next day (Day 1), test materials, reference substances [MAP (100 μg/ml), MAP (50 μg/ml) and niacinamide (400 μg/ml, 250 μg/ml and 80 μg/ml, based on PMC7950568 and PMC8622652)] and negative control (H₂O) were added, and cell culture was pursued for three additional days, afterward the cell culture conditioned medium was used for type I & IV collagen quantification, while cells were formalin-fixed and type III collagen was measured by direct ELISA. The effect of the test articles on cell numbers was determined using the sulforhodamine B method (Table 3). MAP: magnesium ascorbyl phosphate.

All colorimetric measurements were acquired using multi-mode SpectraMax i3x platform (Molecular Devices, San Jose CA) assisted with SoftMax 7.0.3. PRO. 21 software.

Statistically significant variation was defined as ≥20% deviation from the water control with p value <0.05 calculated using double-tailed t-test.

TABLE 3
Assays performed.

| Target | | Type III | | |
|---|---|---|---|---|
| Quantified | Type I Collagen | Collagen | Type IV Collagen | Cell Numbers |
| Materials | Goat anti-collagen | Biotinylated | Anti-collagen IV | Sulforhodamine |
| and | I antibody (cat. # | anti-type III | α1 capture | B method (Voigt |
| Method | 1310-01; Southern | collagen abs | antibody (cat. # | W. |
| | Biotechnology, | (Southern Bio, | AF6308; R&D | Sulforhodamine |
| | Birmingham, AL) | Birmingham, | Systems, | B assay and |
| | for capture, | AL, cat. # | Minneapolis, MN) | chemosensitivity. |
| | biotinylated anti- | 1330-08), | and biotinylated | Methods Mol |
| | collagen I antibody | streptavidin- | anti-collagen IV | Med. 2005; |
| | (cat. # 1310-08, | HRP and TMB | detection antibody | 110: 39-48.). |
| | Southern | reagents for | (polyclonal, cat. # | |
| | Biotechnology) | detection. | NBP1-26550, | |
| | streptavidin-HRP | | Novus, Centennial, | |
| | and TMB reagents | | CO); streptavidin- | |
| | for detection, | | HRP and TMB | |
| | sandwich ELISA | | reagents. | |
| | protocol per Dobak | | | |
| | et al., 1994; Zhao | | | |
| | et al., 2005). | | | |

### Results and Discussion

Type I collagen. Table 4 reports the effect of different experimental conditions on type I collagen output in normal human neonatal dermal fibroblasts. The results show that both saps had a comparable, statistically significant up-regulatory effect on type I collagen with the optimal (best-performing) dose at 5 mg/ml (0.5%). Niacinamide upregulated type I collagen at the two highest tested concentrations. MAP elicited a strong stimulatory response technically validating the experiment.

TABLE 4
Effect of different experimental conditions on type I collagen
output by normal human neonatal dermal fibroblasts. Statistically
significant stimulation is denoted with ***

| | Type I collagen | |
|---|---|---|
| Test Material | (% Control) | p value |
| H₂O | 100 | 1.000 |
| MB-2024 1.6% | 103 | 0.675 |
| MB-2024 0.5% | 136 | 0.000*** |
| MB-2024 0.18% | 117 | 0.026 |
| DBF-2024 1.6% | 129 | 0.001*** |
| DBF-2024 0.5% | 133 | 0.000*** |
| DBF-2024 0.18% | 118 | 0.021 |
| niacinamide 400 ug/mL | 130 | 0.010*** |
| niacinamide 250 ug/mL | 130 | 0.033*** |
| niacinamide 80 ug/mL | 94 | 0.587 |
| MAP 100 ug/mL | 166 | 0.000*** |
| MAP 50 ug/mL | 148 | 0.000*** |

Type III collagen. Table 5 reports the effect of different experimental conditions on type III collagen output in normal human neonatal dermal fibroblasts. The results show that both saps had a statistically significant up-regulatory effect on type III collagen with the optimal (best-performing) dose at 5 mg/ml (0.5%) for MB-2024 and 16 mg/ml (1.6) for DBF-2024. Niacinamide had no effect in the tested range. MAP elicited a strong stimulatory response technically validating the experiment.

TABLE 5
Effect of different experimental conditions on type III collagen
output by normal human neonatal dermal fibroblasts. Statistically
significant stimulation is denoted with ***

| | Type III collagen | |
|---|---|---|
| Test Material | (% Control) | p value |
| H₂O | 100 | 1.000 |
| MB-2024 1.6% | 191 | 0.020*** |
| MB-2024 0.5% | 257 | 0.004*** |
| MB-2024 0.18% | 128 | 0.512 |
| DBF-2024 1.6% | 424 | 0.000*** |
| DBF-2024 0.5% | 304 | 0.000*** |
| DBF-2024 0.18% | 91 | 0.788 |
| niacinamide 400 ug/mL | 111 | 0.236 |
| niacinamide 250 ug/mL | 106 | 0.443 |
| niacinamide 80 ug/mL | 106 | 0.495 |
| MAP 100 ug/mL | 1874 | 0.000*** |
| MAP 50 ug/mL | 1977 | 0.000*** |

Type IV collagen. Table 6 reports the effect of different experimental conditions on type IV collagen output in normal human neonatal dermal fibroblasts. The results show that both saps had a dose-dependent statistically significant up-regulatory effect on type IV collagen, showing high potency and broad range of effective doses. Niacinamide had no effect. MAP elicited a strong stimulatory response technically validating the experiment.

TABLE 6
Effect of different experimental conditions on type IV collagen
output by normal human neonatal dermal fibroblasts. Statistically
significant stimulation is denoted with ***

| | Type IV collagen | |
|---|---|---|
| Test Material | (% Control) | p value |
| H₂O | 100 | 1.000 |
| MB-2024 1.6% | 286 | 0.000*** |
| MB-2024 0.5% | 267 | 0.000*** |
| MB-2024 0.18% | 132 | 0.000*** |
| DBF-2024 1.6% | 224 | 0.000*** |
| DBF-2024 0.5% | 302 | 0.000*** |
| DBF-2024 0.18% | 137 | 0.000*** |
| niacinamide 400 ug/mL | 104 | 0.292 |
| niacinamide 250 ug/mL | 86 | 0.063 |
| niacinamide 80 ug/mL | 88 | 0.132 |
| MAP 100 ug/mL | 703 | 0.000*** |
| MAP 50 ug/mL | 694 | 0.000*** |

Cell Numbers. Table 7 reports cell numbers in the normal human neonatal dermal fibroblast populations under the experimental conditions used in this Example. The results show strong upregulation of cell proliferation by DBF-2024 at the three tested doses. MB-2024 also showed a statistically significant upregulation at one dose. These results may partially account for the higher collagen readings, as collagen expression is proportional to cell numbers.

TABLE 7
Cell numbers in nHDF populations exposed to different
experimental conditions. Statistically significant
stimulation is denoted with ***

| | Cell Numbers | |
|---|---|---|
| Test Material | (% Control) | p value |
| H₂O | 100 | 1.000 |
| MB-2024 1.6% | 84 | 0.025 |
| MB-2024 0.5% | 145 | 0.000*** |
| MB-2024 0.18% | 116 | 0.022 |
| DBF-2024 1.6% | 140 | 0.000*** |
| DBF-2024 0.5% | 199 | 0.000*** |
| DBF-2024 0.18% | 123 | 0.002*** |
| niacinamide 400 ug/mL | 96 | 0.383 |
| niacinamide 250 ug/mL | 99 | 0.933 |
| niacinamide 80 ug/mL | 99 | 0.990 |
| MAP 100 ug/mL | 139 | 0.000*** |
| MAP 50 ug/mL | 130 | 0.000*** |

### Example 3. Effect of Raw Saps of Musa Balbisiana and Dwarf Blue Field Banana on Collagenase and Elastase Activity, In Vitro

This Example describes the effect of the test materials listed in Table 8 on the activity of human elastase and a generic, broad-range collagenase, in vitro.

### Materials and Methods

TABLE 8
Test materials assayed:

| Test material | Lot # | Form | Identifier |
|---|---|---|---|
| Raw sap of Musa | 20240212-BAL- | Brown | MB-2024 |
| Balbisiana | RAW CENTRI | opaque | |
| | | liquids w/ | |
| | | particles | |
| Raw Sap of Dwarf | 20240212-DBF- | Brown | DBF-2024 |
| Blue Field Banana | RAW CENTRI | opaque | |
| (Musa Acuminata × | | liquids w/ | |
| Balbisiana) | | particles | |

Test Materials. Test materials (Table 8) were stored at +4° C. and were diluted in distilled sterile water immediately before testing. All experimental conditions were tested in biological triplicates.

Metalloproteinase activity assay. The activity of the test materials towards collagenase proteolytic activity was examined by adding dilutions of test articles to the Enzchek kit reaction mixture (Molecular Probes/Invitrogen/ThermoFisher). Clostridium collagenase IV (a generic metalloproteinase) was used as the protease and quenched fluorescent gelatin was the substrate. Phenanthroline, a potent collagenase inhibitor was used as positive control at 1000 μM. The kinetics of the release of the digested, fluorescent gelatin were measured at excitation/emission wavelengths of 485/530 nm with the SpectraMax i3x microplate reader. Fluorescence readings were adjusted by subtracting the values in wells containing no collagenase and were expressed in arbitrary fluorescence units and as % of water-treated controls.

Elastase activity assay. The reaction was initiated by adding 10 μl of 20 μg/ml elastase enzyme (Promega, cat. #V1891, lot #409147) to the assay mixture [10 mM Tris-HCl pH 7.5+/−0.5, 1 mg/ml elastase substrate VIII (Suc-Ala-Ala-Ala-pNA)] containing different experimental conditions, and the reaction kinetics were monitored at room temperature by measuring absorbance at 405 nm in the SpectraMax i3x microplate reader. Sterile distilled water was the negative, and EI III (Calbiochem cat. #324745) was the positive control. The negative control was distilled water. Colorimetric readings were adjusted by subtracting the absorbance in wells containing the same experimental conditions but without elastase and were expressed as % of water control.

Statistical analysis. Statistical significance threshold was established as minimum 20% modulation and p value <0.05 as determined by two-tailed Student test.

### Results and Discussion

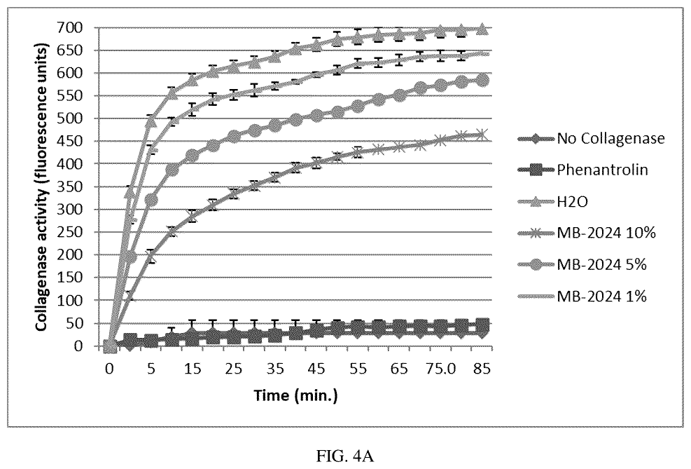

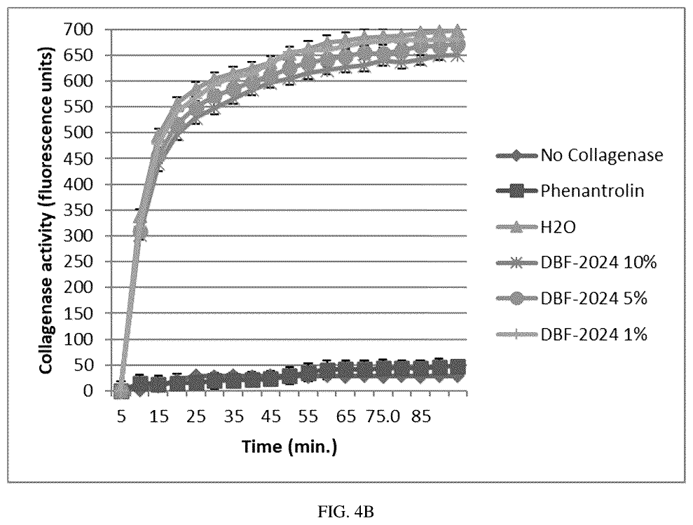

Metalloproteinase activity assay. Table 9 and FIG. 4A and FIG. 4B show that MB-2024 has a moderate inhibitory effect (50% inhibition reached at 10%) on the generic metalloproteinase activity at the tested range, while DBF-2024 has none. Phenanthroline (1 mM) provided total inhibitory activity demonstrating the technical success of the experiment.

TABLE 9
Effect of different experimental conditions on the
collagenase (MP) enzymatic activity at t = 20
min, expressed as the % of the water-treated control.

| | MP activity |
|---|---|
| Test Material | (% CTR) |
| Phenanthroline | 0* |
| H2O | 100 |
| MB-2024 10% | 49*** |
| MB-2024 5% | 72*** |
| MB-2024 1% | 89 |
| DBF-2024 10% | 90 |
| DBF-2024 5% | 94 |
| DBF-2024 1% | 98 |

***statistically significant difference vs. water control (p < 0.05).

Elastase activity assay. Table 10 shows that MB-2024 has a mild to moderate suppressive effect at the tested range, while DBF-2024 has none. Elastase inhibitor EI III (1 μg/ml) provided nearly complete inhibitory activity demonstrating the technical success of the experiment. Two independent experiments were performed.

TABLE 10
Effect of different experimental conditions on the
human elastase enzymatic activity at t = 20
min, expressed as the % of the water-treated control.

| | Elastase activity |
|---|---|
| Test Material | (% Control) |
| H2O | 100 |
| MB-2024 10% | 96 |
| MB-2024 5% | 80 |
| MB-2024 1% | 79 |
| MB-2024 0.5% | 71*** |
| MB-2024 0.1% | 79 |
| DBF-2024 10% | 87 |
| DBF-2024 5% | 86 |
| DBF-2024 1% | 93 |
| DBF-2024 0.5% | 91 |
| DBF-2024 0.1% | 95 |
| El III 1 ug/mL | 9*** |

***statistically significant difference vs. water control (p < 0.05). The table provides averages from two independent experiments.

Conclusion: In both assays in this Example, MB-2024 performed better than DBF-2024, such that MB-2024 provided mild to moderate inhibition of collagenase (MP) enzymatic activity and elastase activity, whereas DBF-2024 showed no inhibition of collagenase (MP) enzymatic activity or inhibition of elastase activity. Note that moderate decrease of collagenase (MP) and elastase activity may be viewed as favorable, because it does not introduce too much of a disruption and is less likely to trigger compensatory mechanisms, which may override any functional gains from the initial inhibition.

### Example 4. RNA Microarray Analysis of Test Materials in Reconstructed Human Skin Model

This Example describes the evaluation of two test materials (Balbisana Sap, Dwarf Blue Field Sap) and two blends of Balbisiana Sap with Allantoin or Hyaluronic Acid in vitro for a gene-level view of changes in the human transcriptome from cultured human 3D skin tissue model EpiDerm-FT™ (MatTek Corporation, Massachusetts, USA). Differential gene expression was quantified using Clariom® S (Life Technologies Corporation, Delaware, USA) assay microarray system method after twenty-four (24) hour incubation with test materials.

Test agents were stored at room temperature until use. Skin tissues were acclimated for 24 hours and later treated topically with Balbisana Sap (1%), Dwarf Blue Field Sap (1%), Balbisana Sap (1%)+Allantoin (0.5%) or Balbisana Sap (1%)+Hyaluronic Acid (0.5%) for gene expression experiments. Total RNA was extracted from tissue samples to perform Global gene expression profiling from 21,448 well-annotated human genes and were analyzed by Affymetrix Human Clariom® S arrays (Life Technologies). Significant expression changes were filtered using fold change: <−1 or >1. The change of expressed genes between treated groups and vehicle control group was confirmed with fold change ≥1 (up-regulated) or ≤−1 (down-regulated). Treatment with Balbisana Sap (1%) regulated 1401 genes (757 up-regulated; 644 down-regulated) compared to untreated (vehicle-only) group. Treatments with Dwarf Blue Field Sap (1%) regulated fewer genes compared to Balbisana Sap with 851 total (460 up-regulated; 391 down-regulated) meeting the <−1 or >1-fold criteria. Combination treatment of Balbisana Sap (1%) with Allantoin (0.5%) produced a higher number (1866) of regulated genes compared to Balbisana Sap-only treatment. In contrast, Balbisana Sap (1%) with Hyaluronic Acid (0.5%) produced a lower number (610) compared to Balbisana Sap-only treatment.

Balbisiana Sap treatment up-regulated the expression of several genes related to skin barrier repair including FGF2 (fibroblast growth factor 2, 126%), which has an important role in cutaneous wound healing (Koike et al., 2020). Dwarf Blue Field Sap and Balbisiana Sap+Allantoin up-regulated PPARG (peroxisome proliferator-activated receptor gamma) by 102% and 63%, respectively. PPAR-Gamma receptor is essential for epidermal differentiation and immune responses (Sobolev et al., 2022). The combination treatment of Balbisiana+Hyaluronic Acid up-regulated TJP2 (tight junction protein 2; 132%), which provides a gate function which controls the paracellular passage of ions and solutes in-between cells (Campbell et a., 2017).

Balbisiana Sap treatment up-regulated the expression of collagen genes including: COL1A1 (collagen, type I, alpha 1; 59%), COL18A1 (collagen, type XVIII, alpha; 20%) and COL20A1 (collagen, type XX, alpha 1; 37%). In contrast, Dwarf Blue Field Sap up-regulated COL22A1 (collagen, type XXII, alpha 1; 66%) and COL6A2 (collagen, type VI, alpha 2; 227%). The up-regulation of collagens in skin is crucial to counteract the natural decline of collagen synthesis, vascularity, skin elasticity and the formation of wrinkles.

None of the treatments had a significant effect on aquaporins (AQP3, AQP9) or hyaluronic acid synthases (HAS1, HAS2), which are crucial for water transport and retention in the epidermis (Patel et al., 2017; Lee et al., 2019). Significant up-regulation of CA4 (carbonic anhydrase IV; 44%) and CRYGS (crystallin gamma S; 31%) was observed by Balbisiana Sap, but not in the Allantoin or HA combinations. Dwarf Blue Field Sap did not significantly up-regulate any of the skin hydration genes analyzed. Combination treatment of Balbisiana Sap with Allantoin up-regulated CA12 (carbonic anhydrase XII) by 44% compared to vehicle group. Interestingly, the combination of Balbisiana Sap with Hyaluronic Acid significantly up-regulated ATP2B4 (ATPase, Ca++transporting, plasma membrane 4) by 97%, which was significantly down-regulated by Balbisiana Sap treatment.

Both Balbisiana and Dwarf Blue Field Sap treatments down-regulated the expression of IL17C by −101% to −153% (Interleukin 17C), which amplifies epithelial inflammation in Th2 and Th17 dominated skin inflammation (Lauffer et al., 2020). Combination treatments of Balbisiana with Allantoin or Hyaluronic Acid further expand this effect suggesting additive or synergistic anti-inflammatory mechanisms. Balbisiana Sap and the combination with Allantoin down-regulated CCL24 (Eotaxin-2) by −104% to −110%. Since CCL24 promotes trafficking of immune cells and activation of pro-fibrotic cells, its regulation might be beneficial for the endothelial dysregulation in systemic sclerosis patients (Levy et al., 2023). Dwarf Blue Field Sap and Balbisiana+Allantoin down-regulated the expression of CXCR6 (chemokine (C—X—C motif) receptor 6) by −72% and −41%, respectively. Down-regulation of this receptor could be beneficial in psoriatic patients since expression is higher in psoriasis than healthy skin (Günther et al., 2012).

Overall, these data show Balbisiana Sap and Dwarf Blue Field Sap can significantly regulate specific genes important in skin barrier, hydration, aging, and inflammation mechanisms.

### INTRODUCTION

The purpose of this study was to evaluate two test materials (Balbisana Sap, Dwarf Blue Field Sap) and two blends of Balbisiana Sap with Allantoin or Hyaluronic Acid in vitro for a gene-level view of changes in the human transcriptome from cultured human 3D skin tissue model EpiDerm-FT™ (MatTek Corporation). Differential gene expression was quantified using Clariom® S assay (Life Technologies) microarray system method after twenty-four (24) hour incubation with test materials.

### Materials and Methods

### Experimental Design

Gene Expression testing in Epiderm-FT™ (MatTek Corporation) skin model. Human 3D full-thickness skin model Epiderm-FT™ (MatTek Corporation) were obtained from MatTek Corp. and acclimated in 6-well plates in vitro using standard cell culture conditions (37° C., 5% CO₂) on Day 1. Tissues were acclimated for 24 hours and randomly separated into five (5) groups (n=3 wells) as indicated in Table 11.

TABLE 11
Treatment groups used in RNA microarray assay

| | Number of |
|---|---|
| Treatment Group | Tissues |
| Vehicle-only | 3 |
| Balbisiana Sap (1%) | 3 |
| Dwarf Blue Field (1%) | 3 |
| Balbisiana Sap (1%) + Allantoin (0.5%) | 3 |
| Balbisiana Sap (1%) + Hyaluronic Acid (0.5%) | 3 |

Tissues were dosed topically with 100 μL of test materials and incubated for 24 hours. After treatment incubation, test materials were removed, and tissues were washed with PBS. Later, total RNA was extracted from a 6 mm biopsy sample and sent to RNA Microarray facility to perform Clariom® S assay (Life Technologies) for transcriptome analysis. Gene differential expression was compared to vehicle-only treated control group.

Test and Control Articles. The test materials used are listed in Table 12. Materials were stored at room temperature (18-22° C.) until use.

TABLE 12
Test Materials information used for tissue treatments.

| Material | Source | Lot/Batch No. |
|---|---|---|
| Balbisiana Sap | Tano Pharmaceuticals, | 20240212-BAL-RAW |
| | Inc. | CENTRI |
| Dwarf Blue Field | | 20240212-DFB-RAW |
| | | CENTRI |
| Allantoin | Talsen Chemicals | ND |
| Sodium Hyaluronate | Bloomage Biotechnology | 20022641 |
| (HMW Hyaluronic | Corporation Limited | |
| Acid) | | |

To prepare the stock formulations, sterilized distilled water (Milli-Q®, Merck KGAA, Germany) was used as diluent or vehicle. Balbisiana Sap or Dwarf Blue Field Sap materials were weighed and mixed with water to make a 1% w/w stock solution. Combination treatment solutions were made similarly, distilled water and Balbisiana Sap were mixed first, then Allantoin or Hyaluronic Acid at 0.5% w/w final concentration were added while blending to ensure even mixing. Vials were sealed and formulations were immediately used for treatments. Tissue assay conditions are described in the Experimental Protocols section.

Tissues. EpiDerm-FT™ (MatTek Corporation) skin tissues were used from human neonatal (EFT-400) origin and purchased as preserved inserts from MatTek Corp. MatTek's EpiDerm-FT™ system (MatTek Corporation) consists of normal, human-derived epidermal keratinocytes (NHEKs) and dermal fibroblasts (HDFs), which have been cultured to form a multilayered, highly differentiated model of the human epidermis/dermis layers. These “ready-to-use” tissues are cultured on specially prepared cell culture inserts using serum-free medium. Tissues were cultured and acclimated for 24 hours before treatments and dosing procedures and tissue groups were identified using appropriate cell culture 6-well plates.

All cell culture procedures were performed at the laboratory facilities which met standards for Biosafety level-2 (BL-2) cell culture room.

Environmental Conditions. Tissue culture incubator was set with a consistent stream of 5% v/v CO₂ with temperature and humidity monitored, controlled, and ranged from 36° C. to 38° C. and 50% to 65%, respectively.

Test Material Administration and Frequency. The test materials were administered topically using a plastic dosing (dispense) pipette of appropriate size. A fixed 100 μL/well dose volume was administered to each cell culture well and incubated for 24 hours at 37° C. and 5% CO₂. After 24 hours, tissues were washed, and total RNA was isolated.

Expression Analysis Parameters. Differential gene expression was analyzed using Transcriptome Analysis Console (TAC) Software (v4.0.1) from Thermo-Fisher Scientific Inc. Data normalization was performed using vehicle-only control group and parameters shown in Table 13.

TABLE 13
Analysis information used for Gene expression analysis.

| Analysis Setting | Parameter |
|---|---|
| Array Type | Clariom_ ® S_Human (Life |
| | Technologies) |
| Summarization Method | Gene Level - SST-RMA |
| Pos vs Neg AUC Threshold | 0.7 |
| Genome version | Hg38 (Homo sapiens) |
| Total Gene Number | 21,448 |
| Gene-Level Fold Change | <−1 or >1 |
| Gene-Level Fold Change | <0.05 |
| ANOVA Method | Empirical Bayes |
| DABG | <0.05 |

### Experimental Protocols

### Study Design: RNA Microarray Gene Cluster-Analysis

Tissues: Full Thickness Reconstructed Human Skin (EpiDerm-FT™, MatTek Corporation) model were purchased from MatTek Corp. (Cat #EFT-400; Lot #37966; Kit: O) Tissue Culture: EpiDerm-FT™ (MatTek Corporation) model will be cultured in serum-free medium from MatTek Corp. (Cat #EPI-400-ASY; Lot #031124GSA) at 37° C. with 5% CO₂. Skin tissues derived from neonatal donors were obtained in 6-well plate format and acclimated for 24 hours.

Dose route/frequency: single topical administration for 24 hours at 37° C. and 5% CO₂.

GLP Compliance: non-GLP.

Protocol:

### Day: 1 (Tissue Conditioning)

1) Filled 8×6-well sterile plates with 2.5 mL of assay medium.
2) Under sterile conditions, removed the sterile gauze from the 6-well plates containing the EpiDerm-FT™ (MatTek Corporation) tissues.
3) Used sterile forceps to remove each insert containing the EpiDerm-FT™ (MatTek Corporation) tissue and removed any remaining shipping agarose that adheres to the outer sides of the insert by gentle blotting on sterile filter paper.
4) Transferred tissues to 6-well plate pre-filled with assay medium.
5) Incubated the 6-well plates at 37° C. and 5% CO₂ for 24 hours.

### Day: 2 (Treatments)

1) After tissue equilibration period, transferred tissue inserts into fresh 6-well plates pre-filled with 2.5 mL media per well.
2) Dosed tissues in the basal compartment (100 μL topically) drop-wise using a positive displacement micropipette with each treatment (n=3).
3) Incubated tissues for 24 hours at 37° C. and 5% CO₂.

### Day 3: RNA Extraction

Reagent: RNAqueous® Kit (Life Technologies, Cat No. AM1912)
1) After 24 hours incubation with test materials, tissues were washed with sterile 1×DPBS and removed remaining wash solution by gently shaking insert and blotting tissue dry with sterile blotting paper.
2) Removed tissues from inserts using forceps and obtained a 6 mm punch biopsy per tissue and added 450 μL of Lysis/Binding solution into a 1 mL homogenization vessel (Kontes Duall ground glass tissue grinder).
3) Dropped tissues into the Lysis/Binding solution and homogenized until formation of homogenous lysate (˜10 strokes).
4) Transferred lysate to a labeled-fresh RNase-free microcentrifuge tube and incubated on ice.
5) Heated a 2.5 mL aliquot of Elution Solution in an RNase-free microcentrifuge tube in a heat block set to 70-80° C.
6) Performed a clarifying centrifugation on lysates for 10 minutes at 14,000 rcf.
7) Mixed 300 μL of 64% Ethanol with 300 μL of tissue lysate in a labeled-fresh RNase-free microcentrifuge and mixed gently but thoroughly by vortexing.
8) Applied the lysate/ethanol mixture (from the previous step) to a Filter Cartridge assembled in a Collection Tube (supplied) and centrifuged for 1 min at 10,000-14,000 rcf.
9) Discarded the flow-through and reused the Collection Tube for the washing steps.
10) Applied 700 μL Wash Solution #1 to the Filter Cartridge. Added the washes through the filter as in the previous step. Discarded the flow-through and reused the tube for subsequent washes.
11) Added 500 μL Wash Solution #2/3. Added the wash solution through the filter as in the previous step.
12) Repeated with a second 500 μL aliquot of Wash Solution #2/3.
13) After discarding the wash solution, continued centrifugation to remove the last traces of wash solution.
14) Transferred the Filter Cartridge into a fresh Collection Tube.
15) Pipetted 50 μL Elution Solution preheated to ˜70-80° C. to the center of the filter. Closed the cap of the tube. Recovered eluate by centrifugation for ˜30 seconds at room temperature (RCF 10,000-14,000×g).

### RNA Microarray Analysis

RNA Samples were analyzed for expression profiling using an Affymetrix Human Clariom® S (Life Technologies) array (21,448 coding genes). Human Gene Cluster analysis was performed focusing on well-annotated genes to perform gene-level expression profiling and assess changes in key genes and pathways. Gene differential expression was quantified using Clariom® S (Life Technologies) assay microarray system method after twenty-four (24) hour incubation with test materials exposure.

Global gene expression profiling from 21,448 well-annotated human genes will be analyzed by Affymetrix Human Clariom® S (Life Technologies) arrays. Significant expression changes were filtered using fold change based on untreated group: >1 (up-regulated) or <−1 (down-regulated).

### Results

Topical treatments were performed in triplicates to human 3D skin tissue model EpiDerm-FT™ (MatTek Corporation) and total RNA was isolated after twenty-four (24) hours incubation.

All RNA samples submitted to gene array passed Quality Control (QC) assessment for microarray analysis. Global gene expression profiling from 21,448 well-annotated human genes was analyzed by Affymetrix Human Clariom® S (Life Technologies) arrays and data visualized using Transcriptome Analysis Console (TAC) Software (Thermo-Fisher Scientific, Inc). Significant expression changes between active-treated and vehicle-only groups were filtered using fold change ≥1 (up-regulated) or ≤−1 (down-regulated) and p Value <0.05 using empirical Bayes ANOVA. Total regulated genes following that criteria are shown in Table 14.

Treatment with Balbisana Sap (1%) regulated 1401 genes (757 up-regulated; 644 down-regulated) compared to untreated (vehicle-only) group. Treatments with Dwarf Blue Field Sap (1%) regulated less genes compared to Balbisana Sap with 851 total (460 up-regulated; 391 down-regulated) meeting the <−1 or >1-fold criteria. Combination treatment of Balbisana Sap (1%) with Allantoin (0.5%) produced a higher number (1866) of regulated genes compared to Balbisana Sap-only treatment. In contrast, Balbisana Sap (1%) with Hyaluronic Acid (0.5%) produced a lower number (610) compared to Balbisana Sap-only treatment.

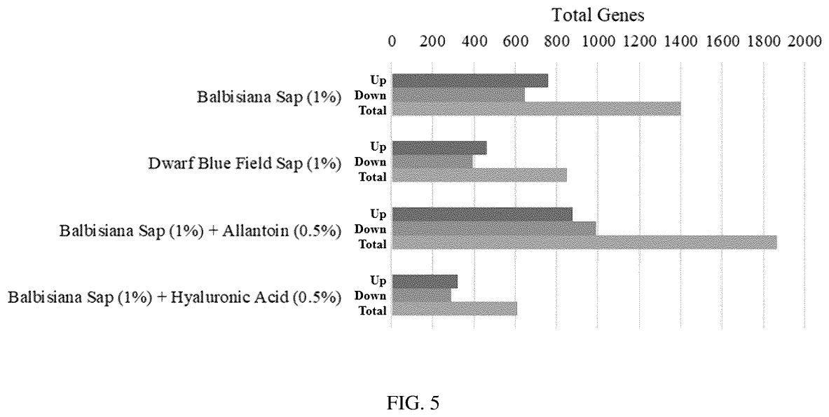

TABLE 14
Summary results of differential gene
expression (also shown in FIG. 5)

| Comparison | Up | Down | Total |
|---|---|---|---|
| Balbisiana Sap (1%) | 757 | 644 | 1401 |
| Dwarf Blue Field (1%) | 460 | 391 | 851 |
| Balbisiana Sap (1%) + Allantoin (0.5%) | 877 | 989 | 1866 |
| Balbisiana Sap (1%) + Hyaluronic Acid (0.5%) | 320 | 290 | 610 |

Differential expression of genes related to skin hydration, skin barrier, skin aging, and inflammation are shown in Tables 15-18.

### Skin Hydration

None of the treatments had a significant effect on aquaporins (AQP3, AQP9) or hyaluronic acid synthases (HAS1, HAS2), which are crucial for water transport and retention in the epidermis (Patel et al., 2017; Lee et al., 2019). Significant up-regulation of CA4 (carbonic anhydrase IV; 44%) and CRYGS (crystallin gamma S; 31%) was observed by Balbisiana Sap but not in the Allantoin or HA combinations. Dwarf Blue Field Sap did not significantly up-regulate any of the skin hydration genes analyzed. Combination treatment of Balbisiana Sap with Allantoin up-regulated CA12 (carbonic anhydrase XII) by 44% compared to vehicle group. Interestingly, the combination of Balbisiana Sap with Hyaluronic Acid significantly up-regulated ATP2B4 (ATPase, Ca++ transporting, plasma membrane 4) by 97%, which was significantly down-regulated by Balbisiana Sap treatment.

TABLE 15
List of Significantly regulated genes related to Skin Hydration

| | Fold-Change vs Vehicle-only group | | | | | | | |
|---|---|---|---|---|---|---|---|---|
| | | | | | Balbisiana | | Balbisiana | |
| | | | Dwarf Blue | | Sap (1%) + | | Sap (1%) + | |
| | Balbisiana | | Field Sap | | Allantoin | | Hyaluronic | |
| | Sap (1%) | | (1%) | | (0.5%) | | Acid (0.5%) | |
| Gene | Fold- | P | Fold- | P | Fold- | P | Fold- | P |
| Symbol | Change | Value | Change | Value | Change | Value | Change | Value |
| ATP2B4 | −1.49 | <0.01 | −1.14 | 0.74 | 1.08 | 0.66 | 1.97 | <0.01 |
| CA12 | 1.16 | 0.16 | 1.36 | 0.07 | 1.59 | 0.02 | 1.31 | 0.47 |
| CA13 | −1.26 | 0.02 | −1.11 | 0.13 | −1.19 | 0.03 | 1.15 | 0.34 |
| CA4 | 1.44 | 0.02 | −1.02 | 0.95 | 1.25 | 0.12 | 1.17 | 0.40 |
| CRYGS | 1.31 | 0.04 | 1.08 | 0.38 | 1.16 | 0.17 | 1.18 | 0.16 |
| PCSK9 | −1.42 | 0.36 | −1.27 | 0.27 | −1.55 | 0.03 | −1.32 | 0.35 |
| PIEZO1 | 1.01 | 0.80 | −1.39 | 0.05 | −1.31 | 0.10 | 1.31 | 0.17 |

Fold-change: Numbers having no sign are up-regulated; numbers having minus sign (−) are down-regulated compared to vehicle-only group. P-value: p < 0.05 = Significantly regulated

### Skin Barrier

Balbisiana Sap treatment up-regulated the expression of several genes related to skin barrier repair including FGF2 (fibroblast growth factor 2, 126%), which has an important role in cutaneous wound healing (Koike et al., 2020). Dwarf Blue Field Sap and Balbisiana Sap+Allantoin up-regulated PPARG (peroxisome proliferator-activated receptor gamma) by 102% and 63%, respectively. PPAR-Gamma receptor is important for epidermal differentiation and immune responses (Sobolev et al., 2022). The combination treatment of Balbisiana+Hyaluronic Acid up-regulated TJP2 (tight junction protein 2; 132%), which provides a gate function which controls the paracellular passage of ions and solutes in-between cells (Campbell et al., 2017).

TABLE 16
List of Significantly regulated genes related to Skin Barrier

| | Fold-Change vs Vehicle-only group | | | | | | | |
|---|---|---|---|---|---|---|---|---|
| | | | | | Balbisiana | | Balbisiana | |
| | | | Dwarf Blue | | Sap (1%) + | | Sap (1%) + | |
| | Balbisiana | | Field Sap | | Allantoin | | Hyaluronic | |
| | Sap (1%) | | (1%) | | (0.5%) | | Acid (0.5%) | |
| Gene | Fold- | P | Fold- | P | Fold- | P | Fold- | P |
| Symbol | Change | Value | Change | Value | Change | Value | Change | Value |
| ABCB1 | 1.69 | 0.02 | 1.22 | 0.24 | 1.00 | 0.97 | 1.19 | 0.60 |
| ABCC2 | −1.12 | 0.30 | −1.03 | 0.68 | 1.06 | 0.72 | −1.09 | 0.32 |
| ABCG4 | 1.15 | 0.32 | −1.69 | 0.12 | −3.86 | 0.04 | 1.52 | 0.60 |
| ADAM15 | −1.03 | 0.91 | −1.01 | 0.46 | −2.76 | 0.02 | −1.41 | 0.44 |
| ADAM17 | −1.48 | 0.01 | −1.25 | 0.15 | −1.56 | 0.02 | −1.22 | 0.24 |
| ADCY10 | −1.38 | 0.17 | −1.53 | 0.08 | −1.66 | 0.02 | −1.58 | 0.05 |
| AGER | 1.21 | 0.29 | 1.04 | 0.87 | 1.88 | 0.02 | −1.07 | 0.64 |
| ALK | 1.20 | 0.51 | −1.27 | 0.05 | −1.06 | 0.50 | −1.11 | 0.50 |
| ANGPT2 | 1.28 | 0.13 | 1.66 | 0.01 | 1.36 | 0.18 | 1.26 | 0.10 |
| ARHGAP29 | 1.14 | 0.50 | −1.09 | 0.74 | 1.07 | 0.75 | 2.17 | 0.03 |
| BCL2 | 1.32 | 0.29 | −1.22 | 0.78 | 1.67 | 0.02 | −1.16 | 0.69 |
| CAV1 | 1.05 | 0.31 | 1.29 | 0.12 | 1.68 | 0.02 | 1.25 | 0.43 |
| CCNA2 | 1.09 | 0.38 | 1.78 | 0.03 | 1.02 | 0.97 | 1.05 | 0.37 |
| CD40 | 2.05 | 0.02 | 2.29 | 0.05 | 2.14 | 0.01 | 2.11 | 0.16 |
| CDC42 | −1.19 | 0.03 | −1.09 | 0.10 | 1.12 | 0.30 | −1.03 | 0.41 |
| CDH13 | −1.73 | 0.02 | −1.56 | 0.03 | −1.72 | 0.02 | −1.55 | 0.16 |
| CDKN1A | −1.82 | 0.01 | −1.43 | 0.16 | −1.20 | 0.11 | −1.23 | 0.54 |
| CTSH | −2.48 | 0.01 | −1.65 | 0.13 | −2.17 | 0.03 | −1.39 | 0.27 |
| CXCL8 | 2.21 | 0.05 | 2.12 | 0.08 | 2.58 | 0.03 | −1.28 | 0.66 |
| CYP26B1 | −1.98 | 0.01 | −1.36 | 0.22 | −1.51 | 0.11 | 1.01 | 0.91 |
| CYP27B1 | −1.36 | 0.18 | −1.41 | 0.12 | −1.75 | 0.02 | −1.34 | 0.04 |
| F10 | −1.15 | 0.38 | 1.41 | 0.13 | 2.35 | 0.05 | 1.77 | 0.25 |
| FERMT3 | −1.22 | 0.93 | 1.43 | 0.02 | −1.50 | 0.08 | −1.26 | 0.51 |
| FGF2 | 2.26 | 0.05 | 1.60 | 0.18 | 1.66 | 0.15 | 1.05 | 0.83 |
| FLNA | 2.16 | 0.11 | 1.92 | 0.15 | 2.77 | 0.04 | 1.95 | 0.29 |
| GBA | −1.10 | 0.59 | −1.71 | 0.06 | −2.34 | 0.01 | 1.39 | 0.12 |
| HGF | 2.53 | 0.05 | 2.63 | 0.25 | 2.26 | 0.15 | −1.07 | 0.88 |
| HMOX1 | −1.86 | 0.09 | −2.17 | 0.02 | −3.44 | <0.01 | 1.71 | 0.11 |
| IL15 | 1.06 | 0.31 | 1.52 | 0.34 | 1.68 | 0.03 | −1.11 | 0.79 |
| IL4 | 1.09 | 0.81 | −1.00 | 0.33 | −1.02 | 0.85 | 1.42 | 0.04 |
| IL6 | 1.73 | 0.02 | 1.50 | 0.03 | 2.06 | 0.01 | −1.01 | 0.93 |
| IL6R | −1.09 | 0.24 | −1.30 | 0.58 | 1.26 | 0.29 | 1.61 | 0.01 |
| IQGAP1 | −1.26 | 0.03 | −1.18 | 0.27 | 1.07 | 0.89 | −1.05 | 0.70 |
| MAP3K1 | −1.39 | 0.44 | −1.25 | 0.13 | −1.56 | 0.02 | −1.41 | 0.22 |
| MYH9 | −1.60 | 0.01 | −1.12 | 0.99 | −1.20 | 0.84 | −1.32 | 0.41 |
| NEDD4 | −1.12 | 0.43 | 1.29 | 0.08 | 1.24 | 0.03 | −1.24 | 0.15 |
| NFKB1 | 1.34 | 0.08 | 1.17 | 0.27 | 1.58 | 0.04 | 1.07 | 0.86 |
| NFKB2 | 1.69 | 0.06 | 1.33 | 0.20 | 1.73 | 0.04 | 1.06 | 0.72 |
| NRP1 | 2.05 | 0.02 | 1.53 | 0.12 | 1.94 | 0.06 | −1.11 | 0.76 |
| PAK1 | −1.49 | 0.03 | −1.24 | 0.14 | −1.21 | 0.17 | −1.16 | 0.25 |
| PCSK9 | −1.42 | 0.36 | −1.27 | 0.27 | −1.55 | 0.03 | −1.32 | 0.35 |
| PDCD6IP | −1.18 | 0.10 | −1.42 | 0.08 | −1.57 | 0.02 | −1.35 | 0.29 |
| PIK3R1 | −1.55 | 0.02 | −1.26 | 0.44 | −1.85 | 0.04 | −1.33 | 0.13 |
| PLTP | 1.18 | 0.10 | 1.43 | 0.03 | 1.20 | 0.05 | 1.09 | 0.35 |
| POLR2A | −1.37 | 0.09 | −1.45 | 0.03 | −1.02 | 0.84 | 1.02 | 0.44 |
| PPARG | 1.02 | 0.22 | 2.02 | 0.03 | 1.63 | 0.03 | 1.39 | 0.14 |
| PRKCD | −1.40 | 0.01 | −1.30 | 0.05 | −1.55 | 0.01 | 1.06 | 0.94 |
| PRNP | −1.25 | 0.04 | −1.22 | 0.04 | −1.67 | 0.02 | 1.36 | 0.43 |
| PROS1 | −2.04 | <0.01 | −1.54 | 0.07 | −1.48 | 0.02 | −1.60 | 0.05 |
| PSORS1C2 | −1.14 | 0.20 | −2.10 | 0.12 | −10.36 | 0.03 | −1.60 | 0.69 |
| PTGS2 | 2.08 | 0.05 | 1.42 | 0.19 | 3.09 | 0.03 | −1.15 | 0.84 |
| PTK2B | −1.72 | 0.56 | −1.32 | 0.23 | −2.48 | 0.01 | −1.38 | 0.96 |
| SEMA3A | 1.65 | 0.02 | 1.28 | 0.65 | −1.14 | 0.70 | 1.33 | 0.61 |
| SETD2 | −1.31 | 0.02 | −1.26 | 0.04 | −1.20 | 0.17 | −1.03 | 0.45 |
| SHPK; | −1.35 | 0.24 | 1.22 | 1.00 | 1.16 | 0.86 | 2.03 | <0.01 |
| TRPV1 | | | | | | | | |
| SLC9A3R1 | −1.50 | 0.10 | −1.23 | 0.13 | −2.25 | 0.05 | 1.15 | 0.79 |
| SLIT2 | 2.75 | 0.02 | 2.05 | 0.06 | 2.42 | 0.03 | 1.70 | 0.51 |
| SRC | −1.04 | 0.44 | 1.17 | 0.22 | 1.31 | 0.09 | 1.62 | <0.01 |
| STK39 | −2.32 | 0.05 | −1.50 | 0.09 | −1.80 | 0.04 | −1.44 | 0.45 |
| SUPT16H | 1.15 | 0.14 | 1.36 | 0.05 | 1.02 | 0.31 | 1.04 | 0.75 |
| TAF1 | 1.54 | 0.02 | 1.45 | 0.03 | 1.52 | 0.01 | 1.25 | 0.17 |
| TARDBP | −1.04 | 0.72 | 1.06 | 0.43 | −1.08 | 0.47 | −1.34 | 0.04 |
| TCTN1 | 1.52 | 0.03 | 1.38 | 0.04 | 2.18 | 0.01 | 1.25 | 0.22 |
| TERT | −1.06 | 0.38 | 1.18 | 0.26 | 1.48 | 0.02 | 1.20 | 0.16 |
| TGFB1 | −2.01 | 0.03 | 1.00 | 0.49 | 1.06 | 0.73 | 1.13 | 0.64 |
| TGFB3 | −1.01 | 0.92 | 1.44 | 0.13 | 1.90 | 0.02 | −1.20 | 0.82 |
| TJP2 | 1.25 | 0.65 | −1.06 | 0.41 | −1.07 | 0.49 | 2.32 | 0.01 |
| TNF | −1.43 | 0.15 | −1.52 | 0.12 | 2.66 | 0.04 | −1.03 | 0.75 |
| TNFRSF1A | −1.56 | 0.01 | −1.20 | 0.46 | −1.49 | 0.02 | −1.31 | 0.17 |
| TP53 | −1.41 | 0.46 | −1.32 | 0.68 | −1.27 | 0.71 | −1.79 | 0.05 |
| TRPV1 | 1.46 | 0.31 | 1.28 | 0.05 | 1.15 | 0.62 | 1.41 | 0.20 |
| TYRO3 | 1.10 | 0.63 | −1.18 | 0.17 | −1.60 | 0.01 | 1.56 | 0.01 |
| UTRN | −1.30 | 0.93 | 1.02 | 0.33 | 1.54 | 0.04 | −1.12 | 0.64 |
| VDR | −1.62 | 0.04 | −1.06 | 0.82 | −1.82 | 0.02 | −1.04 | 0.77 |

Fold-change: Numbers having no sign are up-regulated; numbers having minus sign (−) are down-regulated compared to vehicle-only group. P-value: p < 0.05 = Significantly regulated

### Skin Aging

Balbisiana Sap treatment up-regulated the expression of collagen genes including: COL1A1 (collagen, type I, alpha 1; 59%), COL18A1 (collagen, type XVIII, alpha; 20%) and COL20A1 (collagen, type XX, alpha 1; 37%). In contrast, Dwarf Blue Field Sap up-regulated COL22A1 (collagen, type XXII, alpha 1; 66%) and COL6A2 (collagen, type VI, alpha 2; 227%).

The up-regulation of collagens in skin are crucial to counteract the natural decline of collagen synthesis, vascularity, skin elasticity and the formation of wrinkles.

TABLE 17
List of Significantly regulated genes related to Skin Aging

| | Fold-Change vs Vehicle-only group | | | | | | | |
|---|---|---|---|---|---|---|---|---|
| | | | | | Balbisiana | | Balbisiana | |
| | | | Dwarf Blue | | Sap (1%) + | | Sap (1%) + | |
| | Balbisiana | | Field Sap | | Allantoin | | Hyaluronic | |
| | Sap (1%) | | (1%) | | (0.5%) | | Acid (0.5%) | |
| Gene | Fold- | P | Fold- | P | Fold- | P | Fold- | P |
| Symbol | Change | Value | Change | Value | Change | Value | Change | Value |
| CAT | −2.60 | 0.01 | −1.80 | 0.20 | −1.38 | 0.07 | −2.97 | 0.04 |
| CAV1 | 1.05 | 0.31 | 1.29 | 0.12 | 1.68 | 0.02 | 1.25 | 0.43 |
| COL18A1- | 1.20 | 0.04 | 1.21 | 0.15 | 1.21 | 0.17 | 1.17 | 0.29 |
| AS1 | | | | | | | | |
| COL1A1 | 1.59 | 0.03 | 1.25 | 0.25 | 1.37 | 0.08 | 1.25 | 0.59 |
| COL20A1 | 1.37 | 0.04 | −1.03 | 0.88 | 1.01 | 0.75 | 1.06 | 0.62 |
| COL22A1 | 1.15 | 0.54 | 1.66 | 0.01 | 1.42 | 0.13 | 1.16 | 0.92 |
| COL4A5 | −1.12 | 0.50 | −1.78 | 0.05 | −1.24 | 0.21 | −1.20 | 0.04 |
| COL6A2 | 3.13 | 0.09 | 3.27 | 0.02 | 2.79 | 0.02 | −1.05 | 0.76 |
| COL9A1 | −1.05 | 0.89 | −1.16 | 0.41 | −1.55 | 0.03 | −1.17 | 0.63 |
| EGR1 | −3.13 | 0.03 | 1.27 | 0.92 | 1.16 | 0.44 | −2.14 | 0.05 |
| TP53 | −1.41 | 0.46 | −1.32 | 0.68 | −1.27 | 0.71 | −1.79 | 0.05 |

Fold-change: Numbers having no sign are up-regulated; numbers having minus sign (−) are down-regulated compared to vehicle-only group. P-value: p < 0.05 = Significantly regulated

### Inflammation

Both Balbisiana and Dwarf Blue Field Sap treatments down-regulated the expression of IL17C by −101% to −153% (Interleukin 17C), which amplifies epithelial inflammation in Th2 and Th17 dominated skin inflammation (Lauffer et al., 2020). Combination treatments of Balbisiana with Allantoin or Hyaluronic Acid further expand this effect suggesting additive or synergistic anti-inflammatory mechanisms. Balbisiana Sap and the combination with Allantoin down-regulated CCL24 (Eotaxin-2) by −104% to −110%. Since CCL24 promotes trafficking of immune cells and activation of pro-fibrotic cells, its regulation might be beneficial for the endothelial dysregulation in systemic sclerosis patients (Levy et al., 2023). Dwarf Blue Field Sap and Balbisiana+Allantoin down-regulated the expression of CXCR6 (chemokine (C—X—C motif) receptor 6) by −72% and −41%, respectively. Down-regulation of this receptor could be beneficial in psoriatic patients since expression is higher in psoriasis than healthy skin (Günther et al., 2012).

TABLE 18
List of Significantly regulated genes related to Skin Inflammation

| | Fold-Change vs Vehicle-only group | | | | | | | |
|---|---|---|---|---|---|---|---|---|
| | | | | | Balbisiana | | Balbisiana | |
| | | | Dwarf Blue | | Sap (1%) + | | Sap (1%) + | |
| | Balbisiana | | Field Sap | | Allantoin | | Hyaluronic | |
| | Sap (1%) | | (1%) | | (0.5%) | | Acid (0.5%) | |
| Gene | Fold- | P | Fold- | P | Fold- | P | Fold- | P |
| Symbol | Change | Value | Change | Value | Change | Value | Change | Value |
| AAK1 | −1.39 | 0.03 | −1.12 | 0.39 | 1.13 | 0.15 | −1.17 | 0.25 |
| ABCB1 | 1.69 | 0.02 | 1.22 | 0.24 | 1.00 | 0.97 | 1.19 | 0.60 |
| ABCF1 | 1.43 | 0.01 | −1.09 | 0.96 | 1.12 | 0.14 | −1.05 | 0.39 |
| ACO1 | 1.52 | 0.06 | 1.63 | 0.05 | 1.97 | 0.03 | 1.07 | 0.61 |
| ACP5 | −3.94 | <0.01 | −2.26 | 0.04 | −2.40 | 0.05 | 1.57 | 0.33 |
| ACVR1 | −1.24 | 0.67 | −1.00 | 0.50 | −1.08 | 0.92 | 1.69 | 0.01 |
| ADAM15 | −1.03 | 0.91 | −1.01 | 0.46 | −2.76 | 0.02 | −1.41 | 0.44 |
| ADAM17 | −1.48 | 0.01 | −1.25 | 0.15 | −1.56 | 0.02 | −1.22 | 0.24 |
| ADIPOR1 | −1.19 | 0.12 | −1.15 | 0.12 | −1.61 | 0.03 | −1.15 | 0.74 |
| AGER | 1.21 | 0.29 | 1.04 | 0.87 | 1.88 | 0.02 | −1.07 | 0.64 |
| AHR | 1.74 | 0.04 | 1.63 | 0.05 | 1.98 | 0.05 | 1.48 | 0.58 |
| AIMP1 | 1.03 | 0.55 | 1.06 | 0.17 | 1.47 | 0.04 | 1.02 | 0.95 |
| AJUBA | −1.38 | 0.14 | −1.67 | 0.22 | −2.42 | 0.01 | −1.21 | 0.84 |
| AKR1C3 | 1.52 | 0.10 | 1.57 | 0.03 | 1.61 | 0.03 | 1.29 | 0.10 |
| ALOX12 | −1.31 | 0.18 | −1.19 | 0.19 | −2.80 | 0.03 | −1.94 | 0.41 |
| ALPK1 | 1.16 | 1.00 | 1.57 | 0.03 | 1.52 | 0.03 | 1.28 | 0.23 |
| ALPL | −1.33 | 0.28 | −1.45 | 0.04 | −1.26 | 0.09 | −1.33 | 0.05 |
| ANGPT2 | 1.28 | 0.13 | 1.66 | 0.01 | 1.36 | 0.18 | 1.26 | 0.10 |
| ANKH | −1.62 | 0.26 | −1.14 | 0.40 | −2.34 | 0.03 | −1.41 | 0.27 |
| ARL15 | −1.35 | 0.06 | −1.35 | 0.15 | −1.07 | 0.44 | −1.47 | 0.01 |
| ASIC3 | −1.78 | 0.05 | −1.39 | 0.29 | −1.74 | 0.12 | −1.39 | 0.59 |
| ATF4 | 1.03 | 0.79 | 1.02 | 0.86 | 1.34 | 0.04 | 1.01 | 0.73 |
| ATP2B1 | 1.38 | 0.12 | 1.25 | 0.15 | 2.15 | 0.02 | 1.13 | 0.71 |
| ATP6V0A2 | −1.37 | 0.07 | 1.01 | 0.11 | −1.28 | 0.04 | −1.17 | 0.24 |
| AXIN1 | 1.43 | 0.04 | 1.13 | 0.46 | −1.01 | 0.95 | 1.13 | 0.15 |
| AZGP1 | 1.31 | 0.26 | −1.15 | 0.12 | −6.03 | 0.02 | 1.73 | 0.75 |
| BCL2 | 1.32 | 0.29 | −1.22 | 0.78 | 1.67 | 0.02 | −1.16 | 0.69 |
| BCR | −1.29 | 0.08 | −1.33 | 0.05 | −2.91 | <0.01 | 1.66 | 0.76 |
| BDKRB1 | 1.82 | 0.02 | 1.58 | 0.07 | 3.74 | <0.01 | 1.05 | 0.66 |
| BDKRB2 | −1.06 | 0.35 | 1.38 | 0.14 | 2.14 | 0.04 | 1.20 | 0.94 |
| BIRC2 | 1.15 | 0.15 | 1.01 | 0.45 | 1.58 | 0.03 | 1.00 | 0.93 |
| BIRC5 | 1.15 | 0.30 | 1.08 | 0.35 | −1.03 | 0.68 | −1.20 | 0.04 |
| BLMH | −1.08 | 0.30 | −1.34 | 0.16 | −6.59 | 0.03 | −1.80 | 0.38 |
| BMP7 | −2.36 | 0.03 | −2.35 | 0.16 | −1.10 | 0.59 | −1.88 | 0.27 |
| BNC1 | −1.75 | 0.02 | −2.05 | 0.01 | −2.36 | 0.01 | −1.41 | 0.32 |
| BRCA1 | −1.36 | 0.08 | 1.15 | 0.34 | 1.11 | 0.27 | −1.40 | 0.04 |
| BRD2 | −1.00 | 0.63 | 1.11 | 0.72 | 1.48 | 0.01 | −1.12 | 0.32 |
| C1R | 1.20 | 0.13 | 1.72 | 0.03 | 2.01 | 0.01 | 1.37 | 0.35 |
| CALR | 1.01 | 0.17 | 1.06 | 0.20 | 1.34 | 0.03 | −1.07 | 0.87 |
| CASP4 | −1.09 | 0.20 | −1.06 | 0.46 | −1.10 | 0.22 | −1.33 | 0.04 |
| CASP6 | −1.71 | 0.01 | −1.09 | 0.54 | −1.42 | 0.06 | 1.03 | 0.80 |
| CAT | −2.60 | 0.01 | −1.80 | 0.20 | −1.38 | 0.07 | −2.97 | 0.04 |
| CAV1 | 1.05 | 0.31 | 1.29 | 0.12 | 1.68 | 0.02 | 1.25 | 0.43 |
| CBFA2T3 | 1.20 | 0.61 | −1.02 | 0.90 | 1.01 | 0.67 | 1.21 | 0.42 |
| CBLL1 | −1.31 | 0.04 | −1.35 | 0.03 | −1.53 | <0.01 | −1.66 | <0.01 |
| CBR3 | 1.45 | 0.73 | 2.27 | 0.06 | 1.85 | 0.02 | 1.23 | 0.66 |
| CCL22 | 1.35 | 0.20 | 1.07 | 0.57 | −1.36 | 0.24 | 1.78 | 0.03 |
| CCL23 | 1.57 | 0.04 | 1.25 | 0.17 | 1.58 | 0.03 | 1.51 | 0.07 |
| CCL24 | −2.04 | 0.02 | −1.18 | 0.09 | −2.10 | 0.01 | −1.21 | 0.33 |
| CCR1 | 1.75 | 0.05 | 1.63 | 0.08 | 1.55 | 0.01 | 2.53 | 0.01 |
| CCR8 | −1.25 | 0.07 | −1.32 | 0.04 | −1.20 | 0.28 | −1.16 | 0.13 |
| CD200 | 1.63 | 0.28 | 1.23 | 0.73 | 1.09 | 0.98 | 4.35 | <0.01 |
| CD274 | 3.49 | 0.02 | 2.94 | 0.14 | 5.62 | 0.01 | 1.80 | 0.58 |
| CD40 | 2.05 | 0.02 | 2.29 | 0.05 | 2.14 | 0.01 | 2.11 | 0.16 |
| CD47 | 1.67 | 0.01 | 1.17 | 0.32 | 1.29 | 0.04 | −1.00 | 0.77 |
| CDC42 | −1.19 | 0.03 | −1.09 | 0.10 | 1.12 | 0.30 | −1.03 | 0.41 |
| CDK5 | −1.27 | 0.19 | −1.72 | 0.16 | −1.62 | 0.03 | −1.43 | 0.11 |
| CDK9 | 1.12 | 0.36 | 1.12 | 0.35 | −1.02 | 0.87 | 1.75 | <0.01 |
| CDKN1A | −1.82 | 0.01 | −1.43 | 0.16 | −1.20 | 0.11 | −1.23 | 0.54 |
| CDKN1B | −1.16 | 0.70 | 1.09 | 0.70 | 1.96 | <0.01 | 1.37 | 0.11 |
| CEACAM5 | −1.12 | 0.14 | −1.24 | 0.13 | −2.01 | 0.03 | 1.55 | 0.77 |
| CERK | 1.09 | 0.80 | −1.17 | 0.27 | −1.83 | <0.01 | 1.63 | 0.16 |
| CISH | −1.50 | 0.08 | −2.24 | 0.11 | −1.55 | 0.05 | −1.26 | 0.47 |
| CLEC2D | 2.05 | 0.04 | 1.53 | 0.05 | 1.15 | 0.37 | 1.38 | 0.46 |
| CLEC4A | 1.68 | 0.02 | 1.26 | 0.11 | 1.54 | 0.06 | 1.20 | 0.14 |
| CMKLR1 | 1.27 | 0.18 | 1.53 | 0.03 | 1.08 | 0.68 | −1.02 | 0.84 |
| COQ10A | 1.46 | 0.83 | 1.74 | 0.04 | 1.85 | 0.02 | 1.63 | 0.07 |
| COX11 | 1.22 | 0.17 | 1.38 | 0.04 | 1.73 | 0.02 | 1.04 | 0.53 |
| CREB1 | −1.16 | 0.13 | −1.42 | 0.03 | −1.05 | 0.18 | 1.28 | 0.99 |
| CRH | 1.18 | 0.26 | 1.17 | 0.16 | 1.22 | 0.48 | 1.38 | 0.03 |
| CRLS1 | 1.14 | 0.12 | 1.63 | 0.04 | 1.26 | 0.11 | 1.35 | 0.19 |
| CSF1 | 1.67 | 0.10 | 1.77 | 0.11 | 4.17 | 0.01 | −1.20 | 0.96 |
| CSF1R | 1.19 | 0.37 | 1.41 | 0.02 | 1.53 | 0.14 | 1.26 | 0.41 |
| CSNK2A2 | −2.99 | 0.06 | −1.48 | 0.07 | −4.63 | 0.01 | −1.27 | 0.59 |
| CTSB | 1.69 | 0.01 | −1.06 | 0.55 | −1.17 | 0.54 | 1.06 | 0.91 |
| CTSH | −2.48 | 0.01 | −1.65 | 0.13 | −2.17 | 0.03 | −1.39 | 0.27 |
| CX3CR1 | 1.07 | 0.63 | 1.03 | 0.73 | 1.28 | 0.10 | 1.41 | 0.03 |
| CXCL1 | 1.45 | 0.10 | 1.80 | 0.07 | 2.06 | 0.03 | −1.71 | 0.79 |
| CXCL10 | 3.67 | 0.12 | 1.51 | 0.56 | 3.83 | 0.04 | 2.28 | 0.57 |
| CXCL8 | 2.21 | 0.05 | 2.12 | 0.08 | 2.58 | 0.03 | −1.28 | 0.66 |
| CXCR6 | −1.29 | 0.07 | −1.72 | 0.01 | −1.41 | 0.02 | −1.22 | 0.19 |
| CYB561 | 1.14 | 0.38 | 1.58 | 0.04 | 1.22 | 0.25 | 1.12 | 0.43 |
| CYP27B1 | −1.36 | 0.18 | −1.41 | 0.12 | −1.75 | 0.02 | −1.34 | 0.04 |
| CYP7B1 | 2.14 | 0.05 | 2.21 | 0.14 | 2.70 | 0.02 | 1.14 | 0.84 |
| CYTH2 | −1.60 | 0.01 | −1.74 | 0.00 | −1.99 | <0.01 | −1.05 | 0.73 |
| DAPK1 | 1.32 | 0.17 | 1.47 | 0.04 | 1.33 | 0.18 | 1.16 | 0.40 |
| DDIT3 | 1.37 | 0.57 | 1.35 | 0.31 | 2.04 | 0.02 | −1.87 | 0.10 |
| DDIT4 | −1.35 | 0.02 | −1.18 | 0.15 | 1.17 | 0.59 | 1.14 | 0.92 |
| DOCK4 | 2.18 | 0.03 | 1.18 | 0.52 | 2.34 | 0.02 | 1.11 | 0.77 |
| DUSP1 | −1.99 | 0.01 | −1.71 | 0.04 | −1.16 | 0.45 | −1.26 | 0.52 |
| DUSP10 | 1.12 | 0.53 | 1.31 | 0.95 | 2.60 | 0.04 | 1.08 | 0.17 |
| DUSP14 | −1.22 | 0.22 | −1.48 | 0.13 | −2.62 | 0.05 | −1.40 | 0.79 |
| DUSP5 | −2.42 | <0.01 | −1.84 | 0.03 | −1.39 | 0.03 | −1.49 | 0.22 |
| DVL2 | 1.09 | 0.20 | 1.18 | 0.17 | 1.20 | 0.11 | 1.39 | 0.01 |
| EFHD2 | −1.47 | 0.19 | −1.48 | 0.16 | −1.75 | 0.04 | 1.06 | 0.44 |
| EGLN1 | 1.25 | 0.18 | 1.23 | 0.52 | 2.23 | 0.01 | 1.41 | 0.25 |
| EGR1 | −3.13 | 0.03 | 1.27 | 0.92 | 1.16 | 0.44 | −2.14 | 0.05 |
| ERAP1 | −2.09 | 0.03 | −1.54 | 0.13 | −1.29 | 0.26 | −1.67 | 0.18 |
| ERLIN1 | −1.12 | 0.19 | −1.09 | 0.86 | −1.37 | 0.04 | −1.12 | 0.62 |
| ETS1 | 1.15 | 0.19 | 1.32 | 0.12 | 2.71 | 0.01 | 1.12 | 0.74 |
| F10 | −1.15 | 0.38 | 1.41 | 0.13 | 2.35 | 0.05 | 1.77 | 0.25 |
| FOXO3 | −1.01 | 0.58 | −1.01 | 0.74 | 1.06 | 0.36 | 1.64 | 0.04 |
| FOXP1 | −1.64 | 0.05 | −1.24 | 0.61 | −1.18 | 0.76 | −1.48 | 0.17 |
| FPR3 | 2.05 | 0.31 | 1.33 | 0.32 | 1.50 | 0.17 | 2.12 | 0.02 |
| FURIN | −1.11 | 0.22 | −1.34 | 0.10 | −3.29 | 0.01 | 1.95 | 0.46 |
| FUS | 1.52 | 0.02 | 1.45 | 0.04 | 1.73 | 0.01 | 1.28 | 0.39 |
| GADD45GIP1 | 1.27 | 0.01 | 1.27 | 0.10 | 1.41 | 0.02 | 1.08 | 0.42 |
| GBP1 | 3.39 | 0.02 | 1.83 | 0.09 | 5.70 | <0.01 | 1.37 | 0.42 |
| GCH1 | 1.50 | 0.14 | 1.19 | 0.71 | 1.51 | 0.05 | 1.07 | 0.68 |
| GDA | −1.06 | 0.43 | −1.79 | 0.07 | −5.79 | 0.02 | 1.16 | 0.88 |
| GLO1 | −1.20 | 0.22 | 1.03 | 0.93 | −1.13 | 0.42 | −1.36 | 0.04 |
| GLS | −1.41 | 0.02 | −1.10 | 0.38 | −1.60 | <0.01 | −1.41 | 0.03 |
| GPER1 | 1.03 | 0.97 | −1.00 | 0.61 | 1.33 | 0.05 | −1.00 | 0.17 |
| GPS2 | −1.79 | 0.03 | −1.12 | 0.20 | −1.43 | 0.05 | −1.38 | 0.28 |
| GRM2 | 1.43 | 0.23 | 1.4 | 0.08 | 1.28 | 0.27 | 1.71 | 0.04 |
| GSK3A | −1.42 | 0.11 | −1.18 | 0.11 | −1.69 | 0.03 | 1.11 | 0.50 |
| GSK3B | −1.31 | 0.12 | −1.33 | 0.03 | −1.15 | 0.23 | 1.00 | 0.74 |
| GSTM1 | −1.16 | 0.25 | 1.41 | 0.11 | 1.39 | 0.02 | −1.16 | 0.26 |
| GTF2I | −1.01 | 0.39 | 1.01 | 0.22 | −1.56 | <0.01 | −1.15 | 0.11 |
| GZMB | 1.49 | 0.04 | 1.50 | 0.06 | 1.12 | 0.63 | 1.48 | 0.16 |
| HDAC1 | −1.33 | 0.06 | −1.48 | 0.11 | −1.66 | 0.05 | −1.22 | 0.50 |
| HDAC2 | −1.16 | 0.19 | −1.25 | 0.18 | −1.65 | <0.01 | −1.26 | 0.20 |
| HDAC5 | −1.13 | 0.27 | −1.10 | 0.48 | −1.51 | 0.02 | 1.56 | 0.35 |
| HERPUD1 | −1.34 | 0.14 | 1.11 | 0.76 | 1.54 | 0.01 | −2.34 | 0.00 |
| HGF | 2.53 | 0.05 | 2.63 | 0.25 | 2.26 | 0.15 | −1.07 | 0.88 |
| HK1 | −1.44 | 0.04 | −1.19 | 0.07 | −1.24 | 0.07 | −1.12 | 0.33 |
| HMOX1 | −1.86 | 0.09 | −2.17 | 0.02 | −3.44 | <0.01 | 1.71 | 0.11 |
| HPGD | −1.86 | 0.19 | −1.68 | 0.12 | −10.10 | 0.04 | −1.52 | 0.64 |
| HRH1 | −2.04 | 0.01 | −1.39 | 0.64 | −1.50 | 0.20 | 1.05 | 0.22 |
| HSP90AA1 | −1.09 | 0.18 | −1.31 | 0.04 | −1.55 | 0.02 | −1.58 | 0.01 |
| HSPA4 | 1.00 | 0.74 | 1.09 | 0.30 | −1.44 | 0.05 | −1.10 | 0.52 |
| HTR2A | 1.00 | 0.53 | 2.68 | 0.03 | 3.86 | 0.01 | 1.43 | 0.45 |
| IFIH1 | 1.83 | 0.19 | 1.42 | 0.53 | 1.85 | 0.02 | 1.52 | 0.46 |
| IGFBP5 | 1.02 | 0.16 | 2.06 | 0.04 | 2.20 | 0.03 | 1.75 | 0.21 |
| IL15 | 1.06 | 0.31 | 1.52 | 0.34 | 1.68 | 0.03 | −1.11 | 0.79 |
| IL15RA | 2.20 | 0.01 | 1.49 | 0.12 | 1.26 | 0.04 | 1.10 | 0.48 |
| IL17C | −2.01 | 0.05 | −2.53 | 0.02 | −3.18 | 0.01 | −3.47 | 0.01 |
| IL18BP | 1.26 | 0.11 | 1.27 | 0.04 | 1.20 | 0.20 | 1.09 | 0.93 |
| IL1F10 | −1.01 | 0.43 | −1.19 | 0.20 | −2.32 | 0.02 | 1.31 | 0.48 |
| IL20 | −1.59 | 0.14 | −4.02 | 0.02 | −2.09 | 0.18 | −3.38 | 0.20 |
| IL23A | 3.53 | <0.01 | 1.19 | 0.83 | 2.66 | 0.03 | −1.06 | 0.80 |
| IL36B | 1.06 | 0.21 | −1.71 | 0.13 | −2.65 | 0.04 | 1.13 | 0.83 |
| IL37 | 1.85 | 0.44 | −1.02 | 0.15 | −4.81 | 0.04 | 1.85 | 0.88 |
| IL4 | 1.09 | 0.81 | −1.00 | 0.33 | −1.02 | 0.85 | 1.42 | 0.04 |
| IL4R | 1.03 | 0.89 | −1.11 | 0.49 | −1.99 | <0.01 | 1.22 | 0.93 |
| IL6R | −1.09 | 0.24 | −1.30 | 0.58 | 1.26 | 0.29 | 1.61 | 0.01 |
| IL7 | 1.22 | 0.63 | 1.58 | 0.09 | 1.79 | 0.02 | 1.37 | 0.16 |
| IPMK | 1.15 | 0.11 | 1.30 | 0.09 | 1.71 | 0.03 | 1.36 | 0.23 |
| IRF1 | 1.68 | 0.15 | 1.44 | 0.23 | 2.55 | 0.03 | −1.24 | 0.89 |
| KAT5 | −1.74 | <0.01 | −1.24 | 0.35 | 1.17 | 0.24 | 1.32 | 0.12 |
| KCNIP3 | 1.02 | 0.18 | 1.74 | 0.04 | 1.45 | 0.10 | 1.01 | 0.97 |
| KMT2A | 1.03 | 0.44 | 1.05 | 0.34 | 1.87 | 0.04 | 1.36 | 0.81 |
| LEP | 1.08 | 0.72 | 1.20 | 0.15 | 2.64 | <0.01 | 1.05 | 0.90 |
| LGALS13 | 1.32 | 0.13 | 1.14 | 0.84 | 1.33 | 0.04 | 1.32 | 0.05 |
| LGR5 | −1.48 | 0.03 | −1.26 | 0.24 | −1.28 | 0.39 | −1.09 | 0.41 |
| LIAS | −1.37 | 0.02 | −1.02 | 0.23 | 1.02 | 0.89 | −1.54 | 0.02 |
| LPIN2 | 1.56 | 0.11 | 1.43 | 0.66 | 1.33 | 0.24 | 3.33 | <0.01 |
| MAFB | −2.58 | 0.02 | −1.91 | 0.02 | −2.31 | 0.04 | −1.48 | 0.52 |
| MAP1LC3A | −1.59 | 0.06 | −1.41 | 0.02 | −3.19 | <0.01 | −1.08 | 0.53 |
| MAP2K1 | 1.97 | 0.05 | 2.11 | 0.04 | 3.52 | <0.01 | 1.15 | 0.49 |
| MAP2K3 | 1.04 | 0.60 | −1.12 | 0.27 | −1.10 | 0.29 | 2.40 | 0.01 |
| MAP3K14 | −1.13 | 0.38 | 1.03 | 0.60 | 1.45 | 0.05 | 1.10 | 0.89 |
| MAP3K7 | −1.09 | 0.67 | −1.28 | 0.13 | −1.80 | <0.01 | −1.30 | 0.11 |
| MAP3K8 | −1.14 | 0.76 | −1.25 | 0.42 | 1.58 | 0.34 | 2.33 | 0.02 |
| MAPK14 | −1.76 | 0.08 | −1.20 | 0.15 | −2.48 | 0.01 | 1.08 | 0.84 |
| MAPK7 | −1.36 | 0.05 | −1.35 | 0.08 | −1.41 | 0.15 | 1.15 | 0.86 |
| MARCKS | 1.06 | 0.28 | 1.20 | 0.39 | 1.62 | 0.04 | 1.11 | 0.64 |
| MBD2 | −1.31 | 0.03 | −1.21 | 0.23 | −1.10 | 0.38 | −1.22 | 0.43 |
| MEF2A | −1.15 | 0.80 | 1.00 | 0.48 | 1.51 | 0.01 | 1.06 | 0.71 |
| MICAL2 | −1.54 | 0.02 | 1.28 | 0.29 | 1.09 | 0.63 | 1.28 | 0.18 |
| MID1 | 1.04 | 0.39 | 1.22 | 0.19 | 2.57 | 0.01 | 1.76 | 0.28 |
| MIF | 1.29 | 0.09 | 1.57 | 0.06 | 1.83 | 0.01 | 1.36 | 0.31 |
| MMP3 | 1.04 | 0.40 | −1.40 | 0.02 | 1.68 | 0.17 | 1.37 | 0.66 |
| MMP8 | 2.07 | 0.01 | 1.44 | 0.11 | 2.13 | <0.01 | 1.40 | 0.36 |
| MS4A2 | 1.22 | 0.14 | −1.05 | 0.61 | 1.39 | 0.02 | 1.18 | 0.39 |
| MST1R | −1.18 | 0.17 | −1.71 | 0.01 | −1.25 | 0.05 | 1.03 | 0.44 |
| MTHFD1 | 1.18 | 0.09 | 1.34 | 0.05 | 1.43 | 0.02 | 1.06 | 0.60 |
| MYD88 | −2.15 | 0.02 | −1.58 | 0.08 | −2.43 | 0.05 | −1.60 | 0.61 |
| MYDGF | 1.12 | 0.15 | 1.20 | 0.14 | 1.83 | 0.03 | 1.22 | 0.63 |
| MYH9 | −1.60 | 0.01 | −1.12 | 0.99 | −1.20 | 0.84 | −1.32 | 0.41 |
| NAT1 | −1.12 | 0.39 | 1.31 | 0.02 | −1.11 | 0.50 | −1.02 | 0.32 |
| NCOA4 | −1.62 | 0.07 | −1.30 | 0.12 | −1.46 | 0.05 | −1.32 | 0.26 |
| NCR3 | 1.98 | 0.03 | 1.27 | 0.61 | 1.18 | 0.81 | 1.45 | 0.41 |
| NFAT5 | −1.45 | 0.02 | −1.27 | 0.07 | 1.04 | 0.59 | 1.55 | 0.44 |
| NFE2L2 | 1.08 | 0.31 | 1.13 | 0.72 | 1.37 | 0.04 | −1.04 | 0.79 |
| NFKB1 | 1.34 | 0.08 | 1.17 | 0.27 | 1.58 | 0.04 | 1.07 | 0.86 |
| NFKB2 | 1.69 | 0.06 | 1.33 | 0.20 | 1.73 | 0.04 | 1.06 | 0.72 |
| NFKBIA | 1.11 | 0.25 | −1.09 | 0.77 | 1.48 | 0.03 | −1.06 | 0.53 |
| NLRP2 | 1.47 | 0.85 | −1.16 | 0.49 | −1.42 | 0.03 | 1.28 | 0.95 |
| NMI | 1.25 | 0.61 | 1.17 | 0.58 | 2.27 | 0.02 | −1.68 | 0.11 |
| NRG4 | −1.93 | 0.03 | −4.03 | <0.01 | −1.63 | 0.09 | −1.50 | 0.26 |
| NRP1 | 2.05 | 0.02 | 1.53 | 0.12 | 1.94 | 0.06 | −1.11 | 0.76 |
| NSMAF | 1.37 | 0.09 | 1.13 | 0.21 | 1.83 | 0.02 | 1.02 | 0.83 |
| NUAK2 | −3.12 | 0.01 | −2.31 | 0.09 | −1.85 | 0.09 | −1.77 | 0.81 |
| OR1K1 | −1.25 | 0.34 | −1.15 | 0.49 | −1.49 | 0.04 | −1.10 | 0.92 |
| OTULIN | −1.33 | 0.03 | −1.04 | 0.19 | −1.74 | 0.01 | −1.28 | 0.06 |
| P2RX7 | 1.57 | 0.26 | −1.08 | 0.79 | 2.48 | <0.01 | 1.36 | 0.40 |
| PAFAH1B1 | −1.21 | 0.61 | −1.08 | 0.46 | −1.49 | 0.04 | 1.44 | 0.16 |
| PAK1 | −1.49 | 0.03 | −1.24 | 0.14 | −1.21 | 0.17 | −1.16 | 0.25 |
| PCSK9 | −1.42 | 0.36 | −1.27 | 0.27 | −1.55 | 0.03 | −1.32 | 0.35 |
| PDCD1LG2 | 2.37 | 0.06 | 3.87 | 0.08 | 4.79 | 0.01 | 1.54 | 0.69 |
| PDGFC | 1.06 | 0.31 | 1.12 | 0.15 | 2.09 | 0.01 | 1.37 | 0.20 |
| PDIA3 | 1.30 | 0.05 | 1.56 | 0.07 | 1.39 | 0.04 | 1.05 | 0.90 |
| PDZK1 | 1.67 | 0.04 | 1.48 | 0.04 | 1.06 | 0.71 | 1.08 | 0.68 |
| PELI3 | 1.26 | 0.11 | 1.32 | 0.02 | 1.23 | 0.28 | 1.21 | 0.32 |
| PER2 | 1.08 | 0.43 | −1.07 | 0.74 | 2.04 | <0.01 | −1.13 | 0.26 |
| PHACTR1 | 1.40 | 0.18 | 1.39 | 0.11 | 2.50 | 0.02 | −1.05 | 0.90 |
| PIAS1 | −1.63 | <0.01 | −1.60 | <0.01 | −1.75 | <0.01 | 1.01 | 0.96 |
| PIEZO1 | 1.01 | 0.80 | −1.39 | 0.05 | −1.31 | 0.10 | 1.31 | 0.17 |
| PIKFYVE | 2.00 | 0.03 | 1.26 | 0.39 | 1.44 | 0.21 | 1.23 | 0.85 |
| PLCB3 | −1.37 | 0.02 | −1.24 | 0.09 | −1.40 | 0.01 | −1.04 | 0.44 |
| PLD1 | 1.41 | 0.01 | −1.20 | 0.60 | 1.07 | 0.66 | −1.06 | 0.52 |
| PLIN2 | 1.08 | 0.18 | 1.35 | 0.03 | 1.67 | 0.01 | 1.12 | 0.60 |
| POU5F1 | −1.17 | 0.44 | −1.47 | 0.02 | −1.23 | 0.25 | −1.31 | 0.10 |
| PPARG | 1.02 | 0.22 | 2.02 | 0.03 | 1.63 | 0.03 | 1.39 | 0.14 |
| PPP2R2D | −1.40 | 0.15 | −1.76 | 0.01 | −1.19 | 0.07 | 1.17 | 0.45 |
| PPP2R5C | −1.41 | 0.02 | −1.34 | 0.11 | −1.23 | 0.12 | −1.30 | 0.10 |
| PRAME | 1.31 | 0.03 | 1.03 | 0.29 | −1.58 | 0.24 | 1.03 | 0.31 |
| PRKCD | −1.40 | 0.01 | −1.30 | 0.05 | −1.55 | 0.01 | 1.06 | 0.94 |
| PRKCE | 1.52 | 0.04 | 1.17 | 0.12 | 1.52 | 0.03 | −1.23 | 0.83 |
| PRNP | −1.25 | 0.04 | −1.22 | 0.04 | −1.67 | 0.02 | 1.36 | 0.43 |
| PROS1 | −2.04 | <0.01 | −1.54 | 0.07 | −1.48 | 0.02 | −1.60 | 0.05 |
| PSMD4 | −1.07 | 0.40 | −1.08 | 0.26 | −1.38 | 0.01 | −1.13 | 0.12 |
| PTGS1 | 1.36 | 0.25 | 1.76 | 0.04 | 2.18 | 0.09 | 1.64 | 0.58 |
| PTGS2 | 2.08 | 0.05 | 1.42 | 0.19 | 3.09 | 0.03 | −1.15 | 0.84 |
| PTK2B | −1.72 | 0.56 | −1.32 | 0.23 | −2.48 | 0.01 | −1.38 | 0.96 |
| PTX3 | 1.39 | 0.03 | 1.80 | 0.02 | 1.81 | 0.01 | 1.36 | 0.25 |
| PUM2 | 1.19 | 0.19 | 1.18 | 0.20 | 1.17 | 0.12 | 1.46 | 0.04 |
| RAB23 | 1.12 | 0.20 | 2.04 | 0.04 | 1.55 | 0.09 | 1.13 | 0.42 |
| RB1CC1 | −1.01 | 0.45 | −1.55 | 0.04 | −1.28 | 0.03 | 1.29 | 0.66 |
| REL | −1.47 | 0.09 | −1.46 | 0.02 | −1.15 | 0.18 | −1.16 | 0.45 |
| ROR2 | 1.16 | 0.21 | 1.45 | 0.11 | 2.61 | 0.02 | 1.45 | 0.54 |
| RORA | −1.82 | 0.01 | −1.13 | 0.15 | −1.04 | 0.24 | 1.07 | 0.97 |
| SAR1B | 1.67 | 0.02 | 1.50 | 0.06 | 1.46 | 0.16 | 1.29 | 0.11 |
| SAT1 | 1.55 | 0.04 | 1.88 | 0.07 | 2.29 | 0.02 | 1.15 | 0.85 |
| SCUBE1 | 1.37 | 0.02 | −1.05 | 0.82 | 1.02 | 0.85 | 1.03 | 0.55 |
| SCUBE2 | 1.16 | 0.28 | 1.08 | 0.75 | −1.94 | 0.03 | 1.36 | 0.70 |
| SDC2 | 1.09 | 0.26 | 1.23 | 0.15 | 1.80 | 0.05 | 1.12 | 0.79 |
| SEMA3A | 1.65 | 0.02 | 1.28 | 0.65 | −1.14 | 0.70 | 1.33 | 0.61 |
| SEMA4D | −1.79 | 0.03 | 1.03 | 0.99 | 1.11 | 0.60 | 1.64 | <0.01 |
| SENP3 | −1.46 | 0.13 | −1.04 | 0.89 | −1.23 | 0.17 | −1.94 | 0.02 |
| SENP7 | −1.50 | 0.03 | −1.38 | 0.19 | −1.02 | 0.71 | −1.40 | 0.08 |
| SESN2 | −1.10 | 0.90 | −1.31 | 0.35 | 1.03 | 0.46 | 1.48 | 0.04 |
| SETD2 | −1.31 | 0.02 | −1.26 | 0.04 | −1.20 | 0.17 | −1.03 | 0.45 |
| SIGIRR | −1.71 | 0.03 | −1.42 | 0.17 | −1.25 | 0.07 | −1.09 | 0.72 |
| SIRT2 | −1.66 | 0.02 | −1.60 | 0.22 | −1.73 | 0.10 | −1.26 | 0.58 |
| SLC11A2 | 1.89 | 0.03 | 1.45 | 0.17 | 1.23 | 0.08 | 1.32 | 0.32 |
| SLC19A1 | 1.75 | <0.01 | 1.41 | 0.19 | 1.18 | 0.69 | 1.29 | 0.40 |
| SLC20A1 | −1.55 | 0.03 | −1.98 | <0.01 | −1.07 | 0.63 | −2.05 | <0.01 |
| SLC6A4 | −1.16 | 0.86 | −1.04 | 0.92 | −1.34 | 0.04 | −1.02 | 0.79 |
| SLIT2 | 2.75 | 0.02 | 2.05 | 0.06 | 2.42 | 0.03 | 1.70 | 0.51 |
| SLTM | −1.34 | 0.12 | −1.12 | 0.12 | −1.57 | 0.02 | −1.62 | 0.13 |
| SMG1 | 1.15 | 0.18 | 1.10 | 0.20 | 1.63 | 0.01 | 1.25 | 0.19 |
| SMURF1 | −1.46 | 0.41 | −1.30 | 0.10 | −1.01 | 0.38 | 1.81 | 0.02 |
| SOD2 | 1.35 | 0.03 | 1.59 | 0.02 | 1.65 | 0.01 | 1.14 | 0.44 |
| SORBS3 | −1.43 | 0.29 | 1.44 | 0.54 | 1.99 | 0.05 | 1.02 | 0.44 |
| SOS1 | −1.42 | 0.01 | −1.28 | 0.06 | −1.19 | 0.07 | −1.35 | 0.03 |
| SQSTM1 | 1.46 | 0.07 | −1.01 | 0.41 | 1.60 | 0.03 | 1.41 | 0.06 |
| SRC | −1.04 | 0.44 | 1.17 | 0.22 | 1.31 | 0.09 | 1.62 | <0.01 |
| SSH1 | −1.22 | 0.02 | 1.02 | 0.14 | −1.11 | 0.08 | 1.11 | 0.29 |
| STAM | −1.62 | 0.01 | −1.22 | 0.03 | −1.38 | 0.03 | −1.03 | 0.55 |
| SULT1E1 | −1.51 | 0.01 | −1.55 | 0.01 | −1.14 | 0.29 | −2.00 | <0.01 |
| SUMO2 | −1.16 | 0.10 | −1.16 | 0.07 | −1.22 | 0.05 | −1.12 | 0.16 |
| TBX18 | 1.09 | 0.17 | 2.16 | 0.01 | 2.13 | 0.03 | −1.34 | 0.38 |
| TEAD4 | −1.02 | 0.28 | −1.04 | 0.20 | −1.31 | 0.02 | 1.13 | 0.82 |
| TFAP2C | 1.22 | 0.87 | 1.09 | 0.77 | −2.40 | 0.02 | −1.45 | 1.00 |
| TGFB3 | −1.01 | 0.92 | 1.44 | 0.13 | 1.90 | 0.02 | −1.20 | 0.82 |
| THNSL2 | 1.10 | 0.72 | 1.40 | 0.05 | 1.36 | 0.03 | 1.28 | 0.12 |
| TMUB1 | −1.61 | 0.03 | −1.08 | 0.40 | −1.32 | 0.07 | −1.11 | 0.65 |
| TNF | −1.43 | 0.15 | −1.52 | 0.12 | 2.66 | 0.04 | −1.03 | 0.75 |
| TNFAIP3 | 1.21 | 0.12 | 1.11 | 0.31 | 1.81 | 0.02 | −1.01 | 0.83 |
| TNFSF11 | −3.95 | <0.01 | −1.59 | 0.04 | −4.36 | <0.01 | −2.12 | 0.10 |
| TNIP1 | 1.85 | 0.04 | 1.34 | 0.40 | 1.48 | 0.15 | 1.06 | 0.52 |
| TOR2A | 1.38 | 0.02 | 1.37 | 0.05 | 1.09 | 0.95 | 1.24 | 0.35 |
| TP53 | −1.41 | 0.46 | −1.32 | 0.68 | −1.27 | 0.71 | −1.79 | 0.05 |
| TRAF1 | 2.04 | 0.19 | 1.01 | 0.59 | 2.39 | 0.04 | −1.64 | 0.85 |
| TRIB3 | −2.31 | 0.06 | −1.75 | 0.01 | −1.00 | 0.54 | −1.08 | 0.91 |
| TRIM21 | 1.14 | 0.59 | 1.45 | 0.28 | 1.59 | 0.05 | 1.31 | 0.45 |
| TRIM22 | −1.44 | 0.22 | 1.26 | 0.32 | 1.52 | 0.03 | −1.08 | 1.00 |
| TRPA1 | 2.97 | 0.02 | 1.57 | 0.17 | 1.71 | 0.07 | 1.20 | 0.79 |
| TRPM8 | −1.10 | 0.12 | −1.65 | 0.19 | −1.21 | 0.44 | 2.95 | <0.01 |
| TRPV1 | 1.46 | 0.31 | 1.28 | 0.05 | 1.15 | 0.62 | 1.41 | 0.20 |
| TSC22D3 | 1.13 | 0.65 | 1.51 | 0.22 | 3.51 | 0.01 | 1.10 | 0.84 |
| TSPAN17 | −1.10 | 0.25 | −1.27 | 0.12 | −1.71 | 0.03 | 1.41 | 0.25 |
| TSPAN18 | 1.78 | 0.03 | 1.73 | 0.06 | 1.33 | 0.11 | 1.68 | 0.02 |
| TSPAN33 | 1.25 | 0.22 | −1.12 | 0.76 | 1.42 | 0.05 | 1.29 | 0.07 |
| TSPAN5 | −1.12 | 0.25 | 1.17 | 0.80 | −1.54 | 0.02 | 1.12 | 0.98 |
| TXNRD1 | 2.71 | <0.01 | 1.41 | 0.36 | 1.98 | 0.13 | 2.89 | 0.01 |
| TYRO3 | 1.10 | 0.63 | −1.18 | 0.17 | −1.60 | 0.01 | 1.56 | 0.01 |
| UBA1 | −1.01 | 0.15 | −1.18 | 0.80 | −1.79 | 0.30 | 1.17 | 0.05 |
| UBE2C | 1.59 | 0.04 | −1.08 | 0.54 | −1.08 | 0.45 | 1.26 | 0.17 |
| UPF1 | −1.11 | 0.39 | −1.32 | 0.16 | −1.65 | 0.02 | 1.01 | 0.70 |
| USP7 | −1.54 | 0.03 | −1.24 | 0.04 | −1.44 | 0.06 | −1.25 | 0.36 |
| VASN | 1.08 | 0.72 | 1.91 | 0.06 | 2.15 | 0.03 | 1.73 | 0.06 |
| VASP | −1.64 | 0.05 | −1.33 | 0.11 | −1.45 | 0.03 | −1.28 | 0.59 |
| VCAM1 | 1.00 | 0.77 | 1.54 | 0.01 | −1.02 | 0.96 | 1.16 | 0.98 |
| VDR | −1.62 | 0.04 | −1.06 | 0.82 | −1.82 | 0.02 | −1.04 | 0.77 |
| VTN | −1.38 | 0.03 | −1.29 | 0.12 | −1.06 | 0.67 | −1.29 | 0.13 |
| WNT2B | −1.44 | 0.02 | −1.39 | 0.09 | −1.58 | 0.02 | −1.37 | 0.02 |
| WNT4 | 1.69 | 0.01 | 1.36 | 0.08 | 1.20 | 0.29 | 1.42 | 0.05 |
| WWTR1 | −1.32 | 0.06 | −1.19 | 0.16 | −1.41 | 0.03 | 1.73 | 0.04 |
| XBP1 | 1.12 | 0.30 | 1.15 | 0.27 | 1.45 | 0.04 | −1.07 | 0.73 |
| YAP1 | 1.18 | 0.37 | −1.01 | 0.50 | 1.07 | 0.23 | 1.59 | 0.01 |
| YBX1 | 1.53 | 0.01 | 1.46 | 0.01 | 1.61 | 0.01 | 1.23 | 0.31 |
| ZBTB7B | −1.36 | 0.53 | −1.37 | 0.50 | −5.12 | 0.02 | −1.35 | 0.98 |
| ZC3H12D | 1.02 | 0.53 | 1.17 | 0.87 | 1.59 | 0.04 | 1.36 | 0.16 |
| ZEB2 | 1.57 | 0.15 | 1.57 | 0.19 | 2.66 | 0.04 | 1.09 | 0.83 |
| ZFP36 | −1.68 | 0.03 | −1.31 | 0.10 | 1.13 | 0.48 | 1.14 | 0.96 |
| ZFYVE21 | −1.36 | 0.47 | −1.62 | 0.24 | −2.81 | 0.01 | −1.28 | 0.86 |
| ZNF281 | 1.99 | 0.05 | 1.99 | 0.04 | 1.66 | 0.07 | 1.24 | 0.59 |
| ZNF300 | 1.36 | 0.15 | 1.48 | 0.08 | 1.64 | 0.05 | 1.12 | 0.89 |

Fold-change: Numbers having no sign are up-regulated; numbers having minus sign (−) are down-regulated compared to vehicle-only group. P-value: p < 0.05 = Significantly regulated

### REFERENCES

Campbell H K, Maiers J L, DeMali K A. Interplay between tight junctions & adherens junctions. Exp Cell Res. 2017 Sep. 1;358 (1): 39-44. doi: 10.1016/j.yexcr.2017.03.061.
Günther C, Carballido-Perrig N, Kaesler S, Carballido J M, Biedermann T. CXCL16 and CXCR6 are upregulated in psoriasis and mediate cutaneous recruitment of human CD8+ T cells. J Invest Dermatol. 2012 March; 132 (3 Pt 1): 626-34.
Koike, Y., Yozaki, M., Utani, A. et al. Fibroblast growth factor 2 accelerates the epithelial-mesenchymal transition in keratinocytes during wound healing process. Sci Rep 10, 18545 (2020).
Lauffer F, Jargosch M, Baghin V, Krause L, Kempf W, Absmaier-Kijak M, Morelli M, Madonna S, Marsais F, Lepescheux L, Albanesi C, Müller N S, Theis F J, Schmidt-Weber C, Eyerich S, Biedermann T, Vandeghinste N, Steidl S, Eyerich K. IL-17C amplifies epithelial inflammation in human psoriasis and atopic eczema. J Eur Acad Dermatol Venereol. 2020 April;34 (4): 800-809.
Lee J E, Kim Y A, Yu S, Park S Y, Kim K H, Kang N J. 3,6-Anhydro-L-galactose increases hyaluronic acid production via the EGFR and AMPKα signaling pathway in HaCaT keratinocytes. J Dermatol Sci. 2019 November;96 (2): 90-98.
Levy H, Gluschnaider U, Balbir-Gurman A. The Role of CCL24 in Systemic Sclerosis. Rambam Maimonides Med J. 2023 Jul. 31;14 (3): e0016.
Patel R, Kevin Heard L, Chen X, Bollag W B. Aquaporins in the Skin. Adv Exp Med Biol. 2017; 969:173-191.
Sobolev V V, Tchepourina E, Korsunskaya I M, Geppe N A, Chebysheva S N, Soboleva A G, Mezentsev A. The Role of Transcription Factor PPAR-γ in the Pathogenesis of Psoriasis, Skin Cells, and Immune Cells. Int J Mol Sci. 2022 Aug. 26;23 (17): 9708

### Example 5. Clinical Study is to Assess the Efficacy of a Test Product to Change the Characteristics and Improve Hydration of Facial Skin

### Objectives

The first objective of this clinical study was to assess the efficacy of a test product to change the characteristics and improve hydration of facial skin. The second objective was to obtain before and after images. The third objective was to obtain consumer perception of the test product using questionnaires.

### Study Design

Approximately 25 subjects were enrolled in this clinical study to assess the efficacy of a test product to change the characteristics and improve hydration of facial skin, obtain before and after images, and obtain consumer perception of the test product using questionnaires. The following study evaluations were obtained: expert clinical grading, Newtone ColorFace System images, Corneometer, VapoMeter and Cutometer measurements and consumer perception questionnaire.

A study schedule appears in Table 19.

TABLE 19
Study Schedule - “X” indicates procedure performed at indicated time.

| | | | Immediately | |
|---|---|---|---|---|
| | | | Post- | |
| | Conditioning | | Application | |
| | Phase | | (30 Minutes) + | |
| Study Procedures | Day −7 ± 3 Days | Baseline | 10 mins | Week 4 |
| Informed Consent | X | | | |
| Obtained | | | | |
| Inclusion and | X | | | |
| Exclusion Criteria | | | | |
| Verified | | | | |
| Conditioning Phase | X | | | |
| Soap and Soap Diary | | | | |
| Distributed | | | | |
| Conditioning Phase | | X | | |
| Soap Diary | | | | |
| Collected and | | | | |
| Reviewed | | | | |
| Acclimate to | | X | | X |
| Ambient Laboratory | | | | |
| Conditions for | | | | |
| Approximately 15 | | | | |
| Minutes (±5 | | | | |
| minutes) | | | | |
| Expert Clinical | | X | X | X |
| Grading | | | | |
| Newtone ColorFace | | X | X | X |
| System Images | | | | |
| Corneometer | | X | X | X |
| Measurements | | | | |
| VapoMeter | | X | X | X |
| Measurements | | | | |
| Cutometer | | X | X | X |
| Measurements | | | | |
| Consumer | | | | X |
| Perception | | | | |
| Questionnaire | | | | |
| Test Material | | X | | |
| Application | | | | |
| performed by the | | | | |
| Subjects under the | | | | |
| Supervision of a | | | | |
| Technician | | | | |
| The Test Material, | | | X | |
| Use Instructions, and | | | | |
| Daily Diaries | | | | |
| Distributed | | | | |
| The Test Material | | | | X |
| and Daily Diaries | | | | |
| Collected and | | | | |
| Reviewed | | | | |

### Test Materials

Test product. The test product face cream ingredients are shown in Table 20 below.

TABLE 20
Face cream test product

| | | w/w |
|---|---|---|
| Phase | Name | Percentage |
| A | Distilled Water | 40.00% |
| A | Banana Sap (Musa Balbisiana) | 20.00% |
| A | Aloe Vera (200:1 Powder) | 0.10% |
| A | Sodium Phytate (Chelator) | 0.20% |
| B | Glycerin | 1.50% |
| B | Sclerotium Gum | 0.25% |
| C | Jojoba Oil | 4.30% |
| C | Apricot Kernel Oil | 1.50% |
| C | Mango Butter | 15.00% |
| C | Olivem ® 1000 (Hallstar Italia S.r.l. (Italy) | 4.00% |
| C | Stearic Acid | 1.00% |
| C | Sea buckthorn Fruit Oil | 1.50% |
| C | Camellia Seed Oil (has natural Squalene) | 4.00% |
| C | Rosehip Seed oil | 2.00% |
| D | Sodium Ascorbyl Phosphate | 3.00% |
| D | Tocopherol | 0.50% |
| D | Coenzyme Q10 | 0.10% |
| D | Hyaluronic Acid Powder (MMW) | 0.05% |
| D | Phenoxyethanol & Ethylhexylglycerin | 1.00% |
| | (e.g., Euxyl ® 9010 Schulke & Mayr, | |
| | G.M.B.H. (Germany)) | |

### Manufacturing Protocol:

The banana sap was processed through mechanical juicing, followed by pH stabilization and preservation. This process retains the sap's bioactive properties, ensuring consistent efficacy and stability.

The face cream test product was manufactured as follows:

### 1. Preparation of Phase A:

Weigh all ingredients designated for Phase A.

Add the ingredients into a clean, sanitized mixing vessel.

Stir the mixture under low shear using a suitable mixer until the phase is fully homogeneous. Ensure that no particles or unmixed components remain visible.

### 2. Preparation of Phase B:

Weigh all ingredients for Phase B into a secondary clean and sanitized container.

Stir gently to form a uniform slurry.

Slowly add Phase B to Phase A under continuous low-shear stirring. Gradually incorporate to ensure even dispersion of the gum. Note that a full gel may not form immediately, so care must be taken to achieve even distribution.

Heat the combined Phase A/B to 75° C. while stirring continuously.

### 3. Preparation of Phase C:

Weigh all ingredients for Phase C in a separate sanitized container.

Heat Phase C to 75° C. under gentle stirring until completely homogenous.

4. Combining Phase C with Phase A/B:

Add Phase C to the heated Phase A/B mixture.

Stir the combined phases under high shear until a homogeneous and glossy “milk” texture forms.

Once the mixture achieves a uniform appearance, reduce to low shear stirring and allow the product to begin cooling.

### 5. Incorporation of Phase D:

When the temperature of the batch falls below 40° C., add the ingredients of Phase D to the mixture.

Stir thoroughly until Phase D is evenly dispersed throughout the product.

### 6. pH Adjustment:

When the batch temperature falls below 25° C., measure the pH using a calibrated pH meter.

Adjust the pH to the target range of 5.8-6.2 using citric acid. Add the adjusters in small increments to avoid overshooting the desired range.

Stir gently to ensure uniform pH distribution.

Without intending to be limited by theory, it is expected that the various phases in the test product may contribute to improving skin health and appearance as follows:
Phase A: This phase focuses on hydration delivery using banana sap and humectants such as hyaluronic acid to replenish and retain skin moisture.
Phase B: Stabilizers in this phase contribute to consistent emulsion, maintaining uniform distribution of active ingredients.
Phase C: Lipid-rich emollients such as shea butter and squalane are included in phase C to restore skin's lipid barrier, enhancing elasticity and smoothness.
Phase D: Antioxidants and preservatives are included in this phase to improve skin tone and protect the formulation's integrity.
The test product phases A-D described above may aid in effectively and consistently delivering hydration, elasticity, and tone benefits to the skin.

Storage. Prior to study start, the test material was stored at room temperature and humidity.

### Product Use Instructions.

Amount to Use: Apply a pea-sized amount of the test product to the face and neck.

Frequency: Use twice daily: once in the morning and once in the evening.

Application Time: Morning: Apply the cream after cleansing the face and neck, and before applying sunscreen (if using). Evening: Apply the cream after cleansing the face and neck as the final step in a nighttime routine.

How to Apply: Gently massage the cream into the face and neck using upward, circular motions until fully absorbed.

### Study Population

Inclusion Criteria: A subject may be eligible for study participation if all of the following criteria are met:

Subject is female or male between the ages of 18-75;
a) Subject has mild to moderate of the following on the face:
   a. Uneven skin tone
   b. Fine lines
   c. Wrinkles
   d. Dull skin
b) Subject has normal or dry skin type (approximately 50% of each);
c) Male subject agrees to have shaved their whole face within 24 hours of each visit;
d) Subject agrees not to introduce any new cosmetic or toiletry products during the study;
e) Subject agrees to refrain from drinking hot/caffeinated beverages 2 hours prior to each study visit;
f) Subject agrees to stop using all moisturizing products for the duration of the conditioning phase and only use the designated test product during the study phase as instructed;
g) Subject agrees to use the provided soap for all face washing for the duration of the study;
h) Subject is dependable and able to follow directions as outlined in the protocol and anticipates being available for all study visits;
i) Subject is in generally good health;
j) Subject agrees to sign a Photography Release providing consent for the capture of digital images for use in relation to this clinical study;
k) Subject has completed a HIPAA Authorization Form in conformance with 45 CFR Parts 160 and 164;
l) Subject understands and has signed an Informed Consent Form in conformance with 21 CFR Part 50: “Protection of Human Subjects.”

Exclusion Criteria: A subject is not eligible for study participation if any of the following criteria are met:
a) Female subject is pregnant, nursing, planning a pregnancy, or not using adequate birth control;
b) Subject has facial piercings (unless piercings can be removed for all photography);
c) Subject has visible facial tattoos (including eyeliner, eyebrows, lipliner, etc.);
d) Subject has permanent fake eyelashes or eyelash extensions;
e) Subject is currently using or have used in the past week any systemic or topical; corticosteroids, nonsteroid anti-inflammatory drugs, antihistamines, sympathomimetics, and/or vasoconstrictors or any medication that, in the opinion of the Principal Investigator, preclude application of the test material and/or could influence the outcome of the study;
f) Subject has gotten Botox or plan on getting Botox during the study period;
g) Subject has known allergies to skin treatment products or cosmetics, toiletries, and/or topical drugs;
h) Subject has a history of acute or chronic dermatologic, medical, and/or physical conditions which would, in the opinion of the Principal Investigator, preclude application of the test material and/or could influence the outcome of the study;
i) Subject exhibits irritation, tattoos, sunburn, rashes, scratches, burn marks, scarring, etc. on the test site, which might interfere with evaluations of test results;
j) Subject is currently taking any medications which, in the opinion of the Principal Investigator, may interfere with the study;
k) Subject has a history of skin cancer, or is currently undergoing treatment for active cancer of any kind;
l) Subject has diabetes.

Subject Termination and Withdrawal: A subject may be discontinued from study participation at any time if the Principal Investigator or designated medical staff feels that it is not in the subject's best interest to continue. All subjects are free to withdraw from participation at any time, for any reason, specified or unspecified, and without prejudice. Reasonable attempts will be made by the Principal Investigator or designee to provide a reason for subject withdrawals. The reason for the subject's withdrawal from the study will be specified in the subject's source documents and included in the final report.

Randomization. The left and right cheekbone were randomized for instrumentation measurements in accordance with a computer-generated randomization table.

Blinding. Subjects were blinded to the name of the test material. The investigatory staff were not blinded. The test material was labeled with unique study identification and panel codes and subject numbers.

### Study Evaluations

Expert Clinical Grading. Clinical grading of the face were obtained by an expert grader for the following parameters using the scoring scales in Table 21 below.

TABLE 21
Scoring scales:

| | | | | | | | | | |
|---|---|---|---|---|---|---|---|---|---|
| 0 | 1 | 2 | 3 | 4 | 5 | 6 | 7 | 8 | 9 |

Skin Brightness/Luminosity:

| | | | |
|---|---|---|---|
| Total | Moderate | Mild | Total |
| brightness, | Brightness | Brightness | dullness, |
| translucent | | | matte |
| appearance | | | appearance |

Fine Lines (Visual):

| | | | |
|---|---|---|---|
| No Visible | Mildly | Moderately | Extremely |
| Fine Lines | Visible | Visible | Visible |
| | Fine Lines | Fine Lines | Fine Lines |

Wrinkles (Visual):

| | | | |
|---|---|---|---|
| No Visible | Mildly | Moderately | Extremely |
| Wrinkles | Visible | Visible | Visible |
| | Wrinkles | Wrinkles | Wrinkles |

Evenness of Skin Tone (Visual):

| Even | Mildly | Moderately | Extremely |
|---|---|---|---|
| Skin Tone | Uneven | Uneven | Uneven |
| | Skin Tone | Skin Tone | Skin Tone |

Newtone ColorFace System. The Newtone ColorFace System is a 2D imaging system based on a high-resolution camera with specific lighting and different modalities that allows for capture of full-face images. The Newtone ColorFace System includes a high-resolution 24.2M pixel sensor, 4000×6000 pixels image resolution, and automatic point-to-point focusing. Aural positioning support is coupled with head tilt control by real-time visualization and no positioning constraints (no chin, no front support) allowing for complete visualization of the face. Integration of a specific pattern of 48 hues on each image enables the compensation of colorimetric deviations by a specific algorithm. The Newtone ColorFace System includes adjustable lighting, high power white polarized LED flash lighting, and ultraviolet illumination system (365 nm). Additional features include a motorized filter wheel for multimodal acquisitions (cross polarization, parallel and standard) and system rotation plate with indexing and positioning sensors for acquisition at −45° (left), 0° (face) and +45° (right).

Newtone ColorFace System imaging was captured with the eyes closed. Subjects wore a headband to pull back the hair and a cape to cover clothing. Subjects were asked to remove jewelry at the discretion of the technician.

Lighting modes included: Standard, Standard 45 Cross Polarized and Parallel Polarized.

Corneometer. The general appearance of soft, smooth skin depends on the presence of an adequate amount of water in the stratum corneum. The electrical properties of the skin and its capacitance are largely determined by the water content of the horny layer. The Corneometer is an instrument designed to measure changes in the capacitance of the skin resulting from changes in the degree of hydration. It is particularly sensitive to low hydration levels. The Corneometer expresses the capacitance of the skin surface in arbitrary units of skin hydration. Three Corneometer measurements were obtained from the randomized cheekbone at each designated time point.

Vapometer. The VapoMeter is a fully portable instrument for the measurement of TEWL (transepidermal water loss) values and evaporation rates. TEWL is a well-known indicator of the skin's barrier function. The core of the VapoMeter is a sensitive humidity sensor that is inside a cylindrical measurement closed chamber. This chamber is closed by the skin or other measurement surface during the measurement period and is unaffected by ambient airflows. The sensor monitors the increase of relative humidity (RH) inside the chamber during the measurement. The evaporation rate value (g/m²h) is automatically calculated from the RH increase. The chamber is passively ventilated between measurements and the ventilation time is automatically controlled. Two VapoMeter measurements were obtained from the randomized cheekbone at each designated time point.

Cutometer. The Cutometer assesses the mechanical properties of the skin in a non-invasive manner. The instrument measures the vertical deformation of the surface of the skin as it is pulled by a vacuum suction (500 mm Hg) through a small probe aperture. The suction is generated by a variable vacuum pump, and the depth of penetration of the skin into the probe is measured optically with an accuracy of 0.01 mm. The probe is attached to a computer, which controls the vacuum application and plots skin deformation as a function of time. From the resulting curve, a number of variables can be extrapolated including immediate, delayed, and final distention and immediate retraction. These variables estimate the elastic, viscoelastic, and purely viscous behavior of the skin. The following parameters can be measured:
The final distention, Uf, (R0) measured at 10 seconds;
The immediate distention, Ue, measured at 0.1 seconds;
The delayed distention, Uv;
Immediate retraction, Ur.

The deformation parameters are extrinsic parameters dependent on skin thickness. In order to circumvent the measurement of skin thickness, the following ratios are used to evaluate the elastic nature of the skin:

R0: This parameter represents the passive behavior of the skin to force. Decrease in this parameter indicates improvement in firmness.

R2: Gross elasticity, the closer the value is to 1 (100%) the more elastic the curve, very important parameter. R2 represents the elasticity of the skin and its ability to recover its original position. Increase in this parameter indicates improvement in elasticity.

Ur/Ue (R5) is the biological elasticity of the skin. It measures the ability of the skin to regain its initial configuration after deformation. A value of 1 would indicate 100% elasticity. Increase in this ratio indicate improvement in elasticity.

Uv/Ue (R6) is the ratio between delayed and immediate deformation, i.e. it is the viscoelastic to elastic ratio. An increase in the value of this ratio indicates that there has been an increase in the viscoelastic portion of the deformation and/or relative decrease of the elastic part. Decrease in these ratio indicate improvement in elasticity.

Ur/Uf (R7) is a measure of the net elasticity of the skin. Increase in this ratio indicate improvement in elasticity.

Cutometer measurements were obtained for R0, R2, R5, R6, and R7.

Measurements are obtained by holding the probe firmly against the surface of the skin. The vacuum pump suctions the skin into a small opening and the probe measures the height to which the skin is distended.

Measurements are automatically produced in triplicate, by running through three twenty-second cycles. Each cycle is made up of a suction phase, where the skin is sucked into the probe, and a relaxation phase, where the suction stops and the skin is released. Data is electronically imported into the computer, where ratio values are calculated. Readings are displayed and then manually transcribed onto a score sheet.

One Cutometer measurement will be obtained from the randomized cheekbone at each designated time point.

Consumer Perception Questionnaire. An assessment of test material attributes and the effects of a test material can be determined by questioning the treated subject with regard to consumer perception and the efficacy of the test material following use.

### Test Method

Subject Identification. All subjects were initially identified by a permanent identification number. Once the subject meets qualification criteria, a study subject number was assigned. This study subject number was assigned in sequence as subjects were enrolled in the study.

Conditioning Phase Day −7±3 Days. Subjects reported to the testing facility. Informed consent was obtained. Inclusion and exclusion criteria were verified.

Subjects were provided with non-moisturizing soap and a conditioning phase diary. Subjects were instructed to use only the provided non-moisturizing soap for all washing of the face for the duration of the conditioning phase. Subjects were instructed to refrain from use of moisturization products and anti-aging products for the duration of the conditioning phase.

Baseline. Subjects returned to the testing facility with clean faces, free from makeup. Conditioning phase diaries were reviewed for study compliance and collected. Subjects acclimated to ambient laboratory conditions for approximately 15 minutes (±5 minutes).

The following study evaluations were obtained: expert clinical grading, Newtone ColorFace System images and Corneometer, VapoMeter and Cutometer measurements.

Following the Baseline evaluations, subjects applied the test material under the supervision of a technician.

Immediately Post-Application 30 minutes (±10 minutes). Following the first application, the following study evaluations were obtained: expert clinical grading, Newtone ColorFace System images and Corneometer, VapoMeter and Cutometer measurements. The test material, use instructions, and daily diaries were distributed.

Week 4 (final visit). Subjects returned to the testing facility with a clean face free from makeup/product. Subjects acclimated to ambient laboratory conditions for approximately 15 minutes (±5 minutes). The used and unused test material was collected. Daily diaries were reviewed for study compliance and collected.

The following study evaluations were obtained: expert clinical grading, Newtone ColorFace System images and Corneometer, VapoMeter and Cutometer measurements.

### Data Tabulation

The per-protocol (PP) population was the primary population for all statistical analyses. The PP population included all subjects who received treatment and completed the study in general accordance with the protocol. Only the data of completing subjects was included in the statistical analysis.

Paired sample t-test or Wilcoxon Signed rank test depending on the normality (Shapiro-Wilk test at the threshold of 1%) were applied to determine the differences between Baseline, and each post treatment interval for Expert Clinical Grading, Corneometer measurements, Vapometer measurements and Cutometer measurements. Change from baseline, and % of subjects improved were calculated at each post-treatment interval for the above-mentioned parameters.

Statistical significance exists for p-values≤0.05 at 95% significant level.

Questionnaire responses, for which response category comparisons are informative, were analyzed by Z-tests. Z-tests are used to determine statistically significant differences in the proportions of subjects responding positively or negatively to each question offering a range of responses. If applicable, the proportions of subjects choosing the central (neutral) responses were split equally and added to the response proportion of the top and bottom choices. The split proportions are compared by calculation of a Z-Score to determine statistically significant differences. Statistical significance exists for Z-scores greater than or equal to the absolute value of 1.96 at the 95% confidence level.

### Ethics

Ethical Conduct of the study. The study followed established, standardized procedures for clinical testing designed to ensure the wellbeing of clinical study subjects and the generation of reliable study data. The Study Sponsor was responsible for ensuring the study complies with applicable Drug, Cosmetic or Medical Device regulations, which vary by product.

Informed consent. Each subject was given a copy of the Informed Consent Form (ICF) and Photograph Release Form and have the nature and the purpose of the study explained. Prior to entry into the study, the subjects gave voluntary written consent to participate by signing the ICF. The Principal Investigator retained the original signed Informed Consent Form and signed Photograph Release Form in the subject's file and gave a copy of the Informed Consent Form and Photograph Release Form to the subject.

Subject confidentiality. The Principal Investigator ensured that the research subject's confidentiality was maintained. Subjects were identified by a study ID number only. Documents were kept in strict confidence by the Principal Investigator.

### Adverse Events

Definition of Adverse Event. An adverse event is any untoward medical occurrence, whether or not it is considered study related, including death, experienced by a subject. An event may consist of a disease, an exacerbation of a pre-existing illness or condition, an occurrence of an intermittent illness or condition, a set of related symptoms or signs, or a single symptom or sign.

Definition of a serious Adverse Event. A serious adverse event is any event in which the subject is, in the view of the Principal Investigator, at immediate risk of death or persistent or significant incapacity or substantial disruption of the ability to conduct normal life functions. This definition does not include an event that, had it occurred in a more serious form, might have caused death or persistent or significant incapacity or substantial disruption of the ability to conduct normal life functions. Examples of serious adverse events include, but are not limited to death due to any cause, whether or not it is felt to be related to the study; events that require subject hospitalization; or events that result in a congenital anomaly or birth defect.

Documentation and reporting of Adverse Events. All adverse events were promptly recorded and sufficiently documented by the Principal Investigator or designated medical staff in the source documentation and case report form, even if the adverse event was assessed as unlikely to be related to the study by the Principal Investigator or designated medical staff. The Principal Investigator or designated medical staff reported the occurrence of any serious adverse event to the Sponsor's representative within one business day, regardless of the causal relationship to the study, and follow-up with written documentation within three business days. All adverse events, serious or not serious, related or not related to the test material, were summarized and reported in the final report. All adverse events were followed up until resolved, stabilized, the subject is lost to follow-up, or the event is otherwise explained. Any report of dermal irritation or sensory experiences on the daily diary was listed as experimental data in the clinical study report, and was not considered an adverse event unless confirmed in the laboratory by medical staff. Any report of dermal irritation in the questionnaire portion of the study was listed in the clinical study report as consumer perception data, and was not considered an adverse event unless confirmed in the laboratory by medical staff.

### Quality Assurance Parameters

This clinical study was conducted in accordance with the clinical study protocol and SOPs. The clinical study master file was reviewed for compliance with the clinical study protocol, SOPs, and applicable guidelines and regulations by the Principal Investigator and the Quality Assurance representative.

### Compliance Check

Compliance checks were performed upon review of the daily diary. Up to 15% non-compliance of test material use was accepted. Subjects exceeding 15% non-compliance were discontinued from the study.

### Consumer Perception Questionnaire

Q1. How would you rate your satisfaction with the overall results of the test product? SINGLE SELECTION
Extremely satisfied
Very satisfied
Somewhat satisfied
Not satisfied

Q2. How likely are you to purchase the test product?

### SINGLE SELECTION

Very likely
Likely
I don't know
Probably would not buy
Definitely would not buy

Q3. Which of these brands do you currently use?

### MULTIPLE SELECTION

La Mer
Caudalie
Clarins
Osea
Open text answer

Q4. What would you like to see different about the test product?

### MULTIPLE SELECTION

Fragrance
Thickness
Blendability
Consistency
Open text answer

Q5. How would you rate the hydration of your skin after using the test product for 4 weeks, compared to before you started the study?

### SINGLE SELECTION

My skin feels much more hydrated
My skin feels somewhat more hydrated
My skin feels about the same
My skin feels less hydrated

Q6. How would you describe the firmness of your skin after using the test product for 4 weeks, compared to before the study?

### SINGLE SELECTION

My skin feels much firmer
My skin feels somewhat firmer
My skin feels the same
My skin feels less firm

Q7. How have your fine lines and wrinkles changed after 4 weeks of using the test product?

### SINGLE SELECTION

They are much less noticeable
They are somewhat less noticeable
There's no change
They are more noticeable

Q8. How would you describe the overall tone of your skin after 4 weeks of use? SINGLE SELECTION
My skin looks much more even
My skin looks somewhat more even
My skin looks the same
My skin looks less even

Q9. How smooth does your skin feel after using the test product for 4 weeks?

### SINGLE SELECTION

Much smoother
Somewhat smoother
About the same
Less smooth

Q10. Have you noticed a change in your skin's brightness or glow since starting the test product?

### SINGLE SELECTION

Much brighter
Somewhat brighter
No change
Less bright

Q11. Which of these skin concerns did you notice significant improvement in after using the test product?

### MULTIPLE SELECTION

Fine lines
Wrinkles
Skin texture (smoothness)
Skin tone (evenness)
Firmness
Hydration
Redness
Sensitivity
Dullness
Acne or blemishes
Other (Please specify):

Q12. How would you describe the texture of your skin after using the test product for 4 weeks, compared to before?

### SINGLE SELECTION

Much improved
Somewhat improved
No change
Worse than before

Q13. How well has the test product helped reduce any dryness or flakiness you experienced before the study?

### SINGLE SELECTION

Much improved
Somewhat improved
No change
Made it worse

Q14. Please allocate 100 points across the following skin concerns to reflect how well you feel the test product addressed each one for you.

CONSTANT SUM (must total 100 points)
Fine lines and wrinkles
Skin texture and tone (smoothness and evenness)
Firmness
Dullness or uneven skin tone
Acne or blemishes
Other (Please specify):

Q15. How likely are you to recommend test product to a friend or family member?

### SINGLE SELECTION

Extremely likely
Very likely
Somewhat likely
Unlikely
Very unlikely

### Test Results

TABLE 22
Corneometer Data (i)

| Mean Corneometer Measurement | | | Change From Baseline | |
|---|---|---|---|---|
| (Mean ± SD) | | | (Mean ± SD) | |
| | Immediately | | Immediately | |
| | Post | | Post | |
| | Application | | Application | |
| | 30 Minutes | | 30 Minutes | |
| Baseline | (±10 Minutes) | Week 4 | (±10 Minutes) | Week 4 |
| 44.4 ± 10.5 | 53.4 ± 9.2 | 59.3 ± 9.7 | 9.0 ± 6.6 | 14.8 ± 9.5 |
| | | | (p < 0.0001) | (p < 0.0001) |

Increase in Corneometer values indicates improvement.

TABLE 23
Corneometer Data (ii)
Corneometer Measurement

| | Percent of Subjects |
|---|---|
| Visit | with Improvement |
| Immediately Post Application | 95.7% |
| 30 Minutes (±10 Minutes) | |
| Week 4 | 95.7% |

TABLE 24
Vapometer Data (i)

| Mean Vapometer Measurement | | | Change From Baseline | |
|---|---|---|---|---|
| (Mean ± SD) | | | (Mean ± SD) | |
| | Immediately | | Immediately | |
| | Post | | Post | |
| | Application | | Application | |
| | 30 Minutes | | 30 Minutes | |
| Baseline | (±10 Minutes) | Week 4 | (±10 Minutes) | Week 4 |
| 10.8 ± 2.4 | 10.5 ± 2.0 | 10.8 ± 1.7 | −0.3 ± 0.8 | 0.0 ± 1.1 |
| | | | (p = 0.1015) | (p = 0.8354) |

Decrease in Vapometer values indicates improvement.

TABLE 25
Vapometer Data (ii)
Vapometer Measurement

| | Percent of Subjects |
|---|---|
| Visit | with Improvement |
| Immediately Post Application | 69.6% |
| 30 Minutes (±10 Minutes) | |
| Week 4 | 60.9% |

TABLE 26
Expert Clinical Grading Data (i)
Mean Change From Baseline

| Mean Expert Clinical Grading | | | | Mean Change From Baseline | |
|---|---|---|---|---|---|
| (Mean ± SD) | | | | (Mean ± SD) | |
| | | Immediately | | Immediately | |
| | | Post | | Post | |
| | | Application | | Application | |
| | | 30 Minutes | | 30 Minutes | |
| Parameter | Baseline | (±10 Minutes) | Week 4 | (±10 Minutes) | Week 4 |
| Skin | 5.04 ± 0.82 | 4.87 ± 0.97 | 4.22 ± 1.09 | −0.17 ± 0.39 | −0.83 ± 0.65 |
| Brightness/ | | | | (p = 0.1250) | (p < 0.0001) |
| Luminosity | | | | | |
| Fine Lines | 4.83 ± 0.94 | 4.83 ± 0.94 | 4.78 ± 0.95 | 0.00 ± 0.00 | −0.04 ± 0.21 |
| (Visual) | | | | NA | (p = 1.0000) |
| Wrinkles | 4.00 ± 0.90 | 4.00 ± 0.90 | 4.04 ± 0.93 | 0.00 ± 0.00 | 0.04 ± 0.21 |
| (Visual) | | | | NA | (p = 1.0000) |
| Evenness | 4.09 ± 1.00 | 4.09 ± 1.00 | 3.74 ± 1.21 | 0.00 ± 0.00 | −0.35 ± 0.49 |
| of Skin | | | | NA | (p = 0.0078) |
| Tone | | | | | |
| (Visual) | | | | | |

Decrease in Expert clinical grading scores indicates improvement.

TABLE 27
Expert Clinical Grading Data (ii)
Expert Clinical Grading

| | | Percent of |
|---|---|---|
| | | Subjects with |
| Parameter | Visit | Improvement |
| Skin Brightness/ | Immediately Post Application | 17.4% |
| | 30 Minutes (±10 Minutes) | |
| Luminosity | Week 4 | 69.6% |
| Fine Lines (Visual) | Immediately Post Application | 0.0% |
| | 30 Minutes (±10 Minutes) | |
| | Week 4 | 4.3% |
| Wrinkles (Visual) | Immediately Post Application | 0.0% |
| | 30 Minutes (±10 Minutes) | |
| | Week 4 | 0.0% |
| Evenness of Skin | Immediately Post Application | 0.0% |
| | 30 Minutes (±10 Minutes) | |
| Tone (Visual) | Week 4 | 34.8% |

TABLE 28
Cutometer Data (i)

| Mean Cutometer Measurements | | | | Mean Change From Baseline | |
|---|---|---|---|---|---|
| (Mean ± SD) | | | | (Mean ± SD) | |
| | | Immediately | | Immediately | |
| | | Post | | Post | |
| | | Application | | Application | |
| | | 30 Minutes | | 30 Minutes | |
| Parameter | Baseline | (±10 Minutes) | Week 4 | (±10 Minutes) | Week 4 |
| R0 | 0.3842 ± 0.0758 | 0.4113 ± 0.0755 | 0.3533 ± 0.0687 | 0.0272 ± 0.0687 | −0.0309 ± 0.0847 |
| | | | | (p = 0.0711) | (p = 0.0946) |
| R2 | 0.5358 ± 0.1012 | 0.6194 ± 0.1429 | 0.4986 ± 0.0772 | 0.0836 ± 0.1334 | −0.0372 ± 0.1181 |
| | | | | (p = 0.0065) | (p = 0.1449) |
| R5 | 0.3118 ± 0.0849 | 0.3371 ± 0.0942 | 0.3319 ± 0.0673 | 0.0254 ± 0.0885 | 0.0201 ± 0.0759 |
| | | | | (p = 0.1830) | (p = 0.2170) |
| R6 | 0.6007 ± 0.1269 | 0.5290 ± 0.1098 | 0.6262 ± 0.0962 | −0.0717 ± 0.1149 | 0.0256 ± 0.1591 |
| | | | | (p = 0.0067) | (p = 0.4494) |
| R7 | 0.1939 ± 0.0482 | 0.2220 ± 0.0681 | 0.2041 ± 0.0397 | 0.0281 ± 0.0574 | 0.0102 ± 0.0412 |
| | | | | (p = 0.0283) | (p = 0.2478) |

R0—decrease in this ratio indicate improvement in firmness.
R2—increase in R2 indicate improvement in elasticity.
R5—increase in this ratio indicate improvement in elasticity.
R6—decrease in these ratio indicate improvement in elasticity.
R7—increase in this ratio indicate improvement in elasticity.

TABLE 29
Cutometer Data (ii)
Cutometer Measurements

| | | Percent of |
|---|---|---|
| | | Subjects with |
| Parameter | Visit | Improvement |
| R0 | Immediately Post Application | 34.8% |
| | 30 Minutes (±10 Minutes) | |
| | Week 4 | 69.6% |
| R2 | Immediately Post Application | 69.6% |
| | 30 Minutes (±10 Minutes) | |
| | Week 4 | 39.1% |
| R5 | Immediately Post Application | 73.9% |
| | 30 Minutes (±10 Minutes) | |
| | Week 4 | 60.9% |
| R6 | Immediately Post Application | 78.3% |
| | 30 Minutes (±10 Minutes) | |
| | Week 4 | 47.8% |
| R7 | Immediately Post Application | 69.6% |
| | 30 Minutes (±10 Minutes) | |
| | Week 4 | 60.9% |

TABLE 29
Questionnaire Data (i)
Questionnaire Z test (Week 4)

| | Most | Least | | |
|---|---|---|---|---|
| | Favorable | Favorable | | Significantly |
| Question | Percentage | Percentage | Z-Score | Favorable |
| Q1. How would you rate your | 73.9% | 26.1% | 2.29 | Yes |
| satisfaction with the overall | | | | |
| results of the test product? | | | | |
| SINGLE SELECTION | | | | |
| Q2. How likely are you to | 80.4% | 19.6% | 2.92 | Yes |
| purchase the test product? | | | | |
| SINGLE SELECTION | | | | |
| Q5. How would you rate the | 82.6% | 17.4% | 3.13 | Yes |
| hydration of your skin after | | | | |
| using the test product for 4 | | | | |
| weeks, compared to before you | | | | |
| started the study? SINGLE | | | | |
| SELECTION | | | | |
| Q6. How would you describe the | 73.9% | 26.1% | 2.29 | Yes |
| firmness of your skin after using | | | | |
| the test product for 4 weeks, | | | | |
| compared to before the study? | | | | |
| SINGLE SELECTION | | | | |
| Q7. How have your fine lines | 60.9% | 39.1% | 1.04 | No |
| and wrinkles changed after 4 | | | | |
| weeks of using the test product? | | | | |
| SINGLE SELECTION | | | | |
| Q8. How would you describe the | 69.6% | 30.4% | 1.88 | No |
| overall tone of your skin after 4 | | | | |
| weeks of use? SINGLE | | | | |
| SELECTION | | | | |
| Q9. How smooth does your skin | 91.3% | 8.7% | 3.96 | Yes |
| feel after using the test product | | | | |
| for 4 weeks? SINGLE | | | | |
| SELECTION | | | | |
| Q10. Have you noticed a change | 78.3% | 21.7% | 2.71 | Yes |
| in your skin's brightness or glow | | | | |
| since starting the test product? | | | | |
| SINGLE SELECTION | | | | |
| Q12. How would you describe | 91.3% | 8.7% | 3.96 | Yes |
| the texture of your skin after | | | | |
| using the test product for 4 | | | | |
| weeks, compared to before? | | | | |
| SINGLE SELECTION | | | | |
| Q13. How well has the test | 78.3% | 21.7% | 2.71 | Yes |
| product helped reduce any | | | | |
| dryness or flakiness you | | | | |
| experienced before the study? | | | | |
| SINGLE SELECTION | | | | |
| Q15. How likely are you to | 80.4% | 19.6% | 2.92 | Yes |
| recommend test product to a | | | | |
| friend or family member? | | | | |
| SINGLE SELECTION | | | | |

Most Favorable Percentage = % of subjects chose top 2 responses when there are 4 responses or the top 2 responses plus half of neutral when there are 5 responses, and Least Favorable Percent = % of subjects chose bottom 2 responses when there are 4 responses or the bottom 2 responses plus half of neutral when there are 5 responses

TABLE 29
Questionnaire Data (ii)
Q14. Please allocate 100 points across the following skin
concerns to reflect how well you feel the test product
addressed each one for you. CONSTANT SUM
(must total 100 points) -Mean value

| | Skin texture | | | | |
|---|---|---|---|---|---|
| | and tone | | Dullness | | Other |
| Fine lines | (smoothness | | or uneven | Acne or | (Please |
| and wrinkles | and evenness) | Firmness | skin tone | blemishes | specify): |
| 20.87 | 32.17 | 19.57 | 18.26 | 3.04 | 6.09 |

### Discussion of Test Results

The clinical results of the test product described in this Example demonstrate strong and measurable benefits across several key skin health metrics. These results highlight the efficacy of the test product, particularly in hydration, skin brightness, and texture improvement, as detailed below:

### 1. Exceptional Hydration Benefits

Immediate Improvement: The Corneometer measurements showed a 33% increase in skin hydration immediately after application, with hydration levels rising from a baseline mean of 44.4 to 53.4 (p<0.0001).

Sustained Efficacy: After four weeks of use, hydration levels improved further by 50% compared to baseline, reaching a mean of 59.3 (p<0.0001).

The above results in hydration improvement may reflect the combined effects of banana sap and humectants such as hyaluronic acid, which enhance the skin's ability to retain moisture.

User Perception: Over 91% of participants reported noticeable improvements in skin smoothness and hydration after four weeks.

### 2. Skin Brightness and Tone Enhancement

Clinical Grading: Skin brightness and luminosity improved significantly after four weeks, with an average reduction in clinical grading scores from 5.04 to 4.22 (p<0.0001).

Subject Feedback: Nearly 78% of participants noticed an improvement in their skin's brightness or glow, emphasizing the product's efficacy in delivering a more radiant complexion.

Even Skin Tone: Clinical grading showed improvements in evenness of skin tone, with a reduction in grading scores from 4.09 to 3.74 (p=0.0078), and 34.8% of participants reported visible improvement.

### 3. Texture and Smoothness Refinement

User Feedback: 91% of participants rated their skin as smoother after four weeks of use, supported by improvements in texture noted in clinical grading.

Immediate Effect: The unique combination of ingredients provided a noticeable smoothing effect shortly after application, enhancing the skin's feel and appearance.

### 4. Elasticity Support

Immediate Elasticity Boost: The Cutometer results showed significant immediate improvements in skin elasticity, with R2 (elasticity ratio) increasing by 15.6% (p=0.0065) immediately post-application.

Long-Term Elasticity Maintenance: While the primary benefits were observed immediately, elasticity scores were sustained at a high level throughout the study.

Improvements in elasticity (e.g., R2 and R7 metrics) indicate that the test product provides plumping and firming effects, which may be due to the combined hydrating properties of banana sap and lipid-replenishing ingredients such as squalane and mango butter.

### 5. Overall Satisfaction

Positive Perception: 82.6% of participants expressed high satisfaction with their skin's hydration after four weeks, while 80.4% stated they would recommend the product to friends and family.

Skin Texture and Glow: Participants consistently ranked the product's ability to improve skin texture and tone as its most valuable benefits, allocating an average of 32.17 points to this metric in a constant sum evaluation.

The tests results showed significant improvements in luminosity and smoothness, which may be due to banana sap's antioxidant activity and its interaction with brightening agents and emollients in the test product.

### CONCLUSIONS

The example test product was clinically proven to deliver immediate and long-lasting benefits in skin hydration, brightness, texture, and elasticity. These results confirm its ability to improve overall skin health and appearance, providing users with smoother, more radiant, and visibly refreshed skin. Without limitation to theory, these observed beneficial effects may be associated with the combination of ingredients in the test product to deliver complementary and/or synergistic benefits for improving skin health and appearance. The observed beneficial effects may be associated with the combined effects of banana sap and complementary ingredients. For example: Banana sap's hydration-enhancing properties combined with humectants may deliver immediate and long-lasting moisture. Antioxidants present in banana sap may amplify the effects of brightening agents, contributing to improved skin tone and luminosity. Lipid-replenishing emollients in the test product may enhance elasticity and overall skin texture, creating a noticeable plumping effect.

The above disclosure contains various examples of banana stem sap compositions and methods of use thereof. Aspects of these various examples may all be combined with one another, even if not expressly combined in the present disclosure, unless they are clearly mutually exclusive.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

Furthermore, unless it is otherwise clear that a single entity is intended, terms such as “a,” “an,” and “the” are not intended to refer to only a singular entity and include the general class of which a specific example is described for illustration. In addition, unless it is clear that a precise value is intended, numbers recited herein should be interpreted to include variations above and below that number that may achieve substantially the same results as that number, or variations that are “about” the same number. Finally, a derivative as disclosed herein may include a chemically modified molecule that has an addition, removal, or substitution of a chemical moiety of the parent molecule.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein and unless otherwise specified, the term “about” means within plus or minus 10% of a given value or range.

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

Various compositions may be identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A cosmetic skincare composition comprising banana stem sap or an extract thereof and one or more cosmetically acceptable excipients.

2. The cosmetic skincare composition of claim 1, formulated for topical administration.

3. The cosmetic skincare composition of claim 1, formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.

4. The cosmetic skincare composition of claim 1, wherein the banana stem sap or an extract thereof is from one or more members of the genus Musa.

5. The cosmetic skincare composition of claim 1, wherein the banana stem sap or an extract thereof is from a member of the Musa genus selected from M.×alinsanaya R. V. Valmayor, M. azizii Häkkinen, M. barioensis Häkkinen, M. bauensis Häkkinen & Meekiong, M. beccarii N. W. Simmonds, M. boman Argent, M. borneensis Becc., M. bukensis Argent, M. velutina, M. campestris Becc., M. coccinea Andrews, M. exotica R. V. Valmayor, M. fitzalanii F. Muell., M. gracilis Holttum, M. hirta Becc., M. insularimontana Hayata, M. jackeyi W. Hill, M. johnsii Argent, M. lawitiensis Nasution & Supard., M. lokok Geri & Ng, M. lolodensis Cheesman, M. maclayi F. Muell. ex Mikl.-Maclay, M. monticola M. Hotta ex Argent, M. muluensis M. Hotta, M. paracoccinea A. Z. Liu & D. Z. Li, M. peekelii Lauterb., M. salaccensis Zoll. ex Backer, M. textilis Née, M.×troglodytarum L., M. tuberculata M. Hotta, M. violascens Ridl., M. viridis R. V. Valmayor et al., M. voonii Häkkinen, M. ingens N. W. Simmonds, M. acuminata Colla, M. acuminata subsp. zebrina, M. aurantiaca G. Mann ex Baker, M. balbisiana Colla, M. banksii F. Muell., M. basjoo Siebold & Zucc. ex Iinuma, M. cheesmanii N. W. Simmonds, M. chunii Häkkinen, M. griersonii Noltie, M. itinerans Cheesman, M. laterita Cheesman, M. mannii H. Wendl. ex Baker, M. nagensium Prain, M. ochracea K. Sheph., M. ornata Roxb., Musa×paradisiaca L., M. rosea Baker, M. rubinea Häkkinen & C. H. Teo, M. rubra Wall. ex Kurz, M. sanguinea Hook.f., M. schizocarpa N. W. Simmonds, M. siamensis Häkkinen & Rich. H. Wallace, M. sikkimensis Kurz, M. thomsonii (King ex Baker) A. M. Cowan & Cowan, M. truncata Ridl., M. velutina H. Wendl. & Drude, M. yunnanensis Häkkinen & H. Wang, M. zaifui Häkkinen & H. Wang, M. arfakiana Argent, M. arunachalensis A. Joe, Sreejith & M. Sabu, M. celebica Warb. ex K. Schum., M. juwiniana Meekiong, M. kattuvazhana K. C. Jacob, M. lanceolata Warb. ex K. Schum., M. lutea R. V. Valmayor et al., M. sakaiana Meekiong et al., M. shankarii Subba Rao & Kumari, M. splendida A. Chev., M. tonkinensis R. V. Valmayor et al., and M. yamiensis C. L. Yeh & J. H. Chen, and any combinations thereof.

6. The cosmetic skincare composition of claim 1, wherein the one or more cosmetically acceptable excipients comprises one or more fragrances, essential oils, colorants, preservatives, exfoliating agents, anti-aging ingredients, mattefying agents, alcohols, silicones, hydroquinones, or combinations thereof.

7. The cosmetic skincare composition of claim 1, comprising an amount of the banana stem sap or extract thereof effective to increase fibroblast proliferation in the skin, increase PDGF-BB in the skin, increase collagen production in the skin, or any combinations thereof, following administration of the composition to skin of a subject.

8. The cosmetic skincare composition of claim 1, comprising an amount of the banana stem sap or extract thereof effective to reduce wrinkles in the skin, reduce fine lines in the skin, reduce skin laxity, improve skin texture, improve skin tone, reduce visibility of scars, reduce stretch marks, reduce age spots, reduce hyperpigmentation, reduce skin roughness, reduce uneven pigmentation, reduce dullness, increase skin hydration, increase skin elasticity, increase skin firmness, reduce enlarged skin pore size, reduce acne, reduce acne scars, reduce skin damage caused by sun exposure, reduce skin damage caused by exposure to pollution, reduce under-eye skin circles, reduce under-eye skin bags, reduce skin puffiness, increase skin radiance, increase skin glow, reduce skin irritation, reduce skin rashes (e.g. diaper rashes, among others), reduce skin blemishes, promote skin tightening, reduce eczema, or any combinations thereof, following administration of the composition to skin of a subject.

9. A method of treating or preventing a cosmetic skin condition in a subject, the method comprising:
administering to the subject's skin an effective amount of banana stem sap or an extract thereof, or an effective amount of the cosmetic skincare composition of claim 1.

10. The method of claim 9, wherein the subject's skin to which the skincare composition is administered does not comprise a wound.

11. A wound healing composition comprising banana stem sap or an extract thereof and one or more pharmaceutically acceptable excipients.

12. The wound healing composition of claim 11, formulated for topical administration.

13. The wound healing composition of claim 11, formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.

14. The wound healing composition of claim 11, wherein the banana stem sap or an extract thereof is from one or more members of the genus Musa.

15. The wound healing composition of claim 11, wherein the banana stem sap or an extract thereof is from a member of the Musa genus selected from M.×alinsanaya R. V. Valmayor, M. azizii Häkkinen, M. barioensis Häkkinen, M. bauensis Häkkinen & Meekiong, M. beccarii N. W. Simmonds, M. boman Argent, M. borneensis Becc., M. bukensis Argent, M. velutina, M. campestris Becc., M. coccinea Andrews, M. exotica R. V. Valmayor, M. fitzalanii F. Muell., M. gracilis Holttum, M. hirta Becc., M. insularimontana Hayata, M. jackeyi W. Hill, M. johnsii Argent, M. lawitiensis Nasution & Supard., M. lokok Geri & Ng, M. lolodensis Cheesman, M. maclayi F. Muell. ex Mikl.-Maclay, M. monticola M. Hotta ex Argent, M. muluensis M. Hotta, M. paracoccinea A. Z. Liu & D. Z. Li, M. peekelii Lauterb., M. salaccensis Zoll. ex Backer, M. textilis Née, M.×troglodytarum L., M. tuberculata M. Hotta, M. violascens Ridl., M. viridis R. V. Valmayor et al., M. voonii Häkkinen, M. ingens N. W. Simmonds, M. acuminata Colla, M. acuminata subsp. zebrina, M. aurantiaca G. Mann ex Baker, M. balbisiana Colla, M. banksii F. Muell., M. basjoo Siebold & Zucc. ex Iinuma, M. cheesmanii N. W. Simmonds, M. chunii Häkkinen, M. griersonii Noltie, M. itinerans Cheesman, M. laterita Cheesman, M. mannii H. Wendl. ex Baker, M. nagensium Prain, M. ochracea K. Sheph., M. ornata Roxb., Musa×paradisiaca L., M. rosea Baker, M. rubinea Häkkinen & C. H. Teo, M. rubra Wall. ex Kurz, M. sanguinea Hook.f., M. schizocarpa N. W. Simmonds, M. siamensis Häkkinen & Rich. H. Wallace, M. sikkimensis Kurz, M. thomsonii (King ex Baker) A. M. Cowan & Cowan, M. truncata Ridl., M. velutina H. Wendl. & Drude, M. yunnanensis Häkkinen & H. Wang, M. zaifui Häkkinen & H. Wang, M. arfakiana Argent, M. arunachalensis A. Joe, Sreejith & M. Sabu, M. celebica Warb. ex K. Schum., M. juwiniana Meekiong, M. kattuvazhana K. C. Jacob, M. lanceolata Warb. ex K. Schum., M. lutea R. V. Valmayor et al., M. sakaiana Meekiong et al., M. shankarii Subba Rao & Kumari, M. splendida A. Chev., M. tonkinensis R. V. Valmayor et al., and M. yamiensis C. L. Yeh & J. H. Chen, and any combinations thereof.

16. The wound healing composition of claim 11, wherein the composition comprises an amount of the banana stem sap or extract thereof effective to increase fibroblast proliferation in the skin, increase PDGF-BB in the skin, increase collagen production in the skin, or any combinations thereof, following administration of the composition to a wound in skin of a subject.

17. The wound healing composition of claim 11, wherein the composition comprises an amount of the banana stem sap or extract thereof effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems, improve in the healing of skin and tissue damage caused by radiation treatments, reduce scar formation, or any combination thereof, following administration of the composition to a wound in skin of a subject.

18. The wound healing composition of claim 11, wherein the wound is an acute wound, a chronic wound, a cut, an abrasion, a surgical incision, a skin graft, a traumatic wound, a hypertrophic scar, a diabetic ulcer, a venous ulcer, a pressure sore, a surgical wound, a post-operative wound, a burn wound, skin or tissue damage caused by radiation treatments, or any combination thereof.

19. A method of treating a subject for a wound, the method comprising:
administering to the subject's skin an effective amount of the wound healing composition of claim 11.
